(19) United States
(12) Patent Application Publication
(10) Pub. No.: US 2023/0181463 A1
(43) Pub. Date: Jun. 15, 2023

(54) OLEOGEL AND OLEOPASTE COMPOSITIONS AND USES THEREOF

(71) Applicants: Massachusetts Institute of Technology, Cambridge, MA (US); The Brigham and Women's Hospital, Inc., Boston, MA (US)

(72) Inventors: Robert S. Langer, Newton, MA (US); Carlo Giovanni Traverso, Newton, MA (US); Ameya R. Kirtane, Arlington, MA (US); Aniket Wahane, Willimantic, CT (US); Christina Karavasili, Boston, MA (US)

(73) Assignees: Massachusetts Institute of Technology, Cambridge, MA (US); The Brigham and Women's Hospital, Inc., Boston, MA (US)

(21) Appl. No.: 17/925,180

(22) PCT Filed: May 14, 2021
(86) PCT No.: PCT/US2021/032428

§ 371 Date: Nov. 14, 2022

Related U.S. Application Data
(60) Provisional application No. 63/025,488, filed on May 15, 2020.

(51) Int. Cl. A61K 9/06; A61K 9/51; A61K 31/7052; A61K 31/7048; A61K 31/137; A61K 31/4985; A61K 47/14

(52) U.S. Cl. CPC A61K 9/06; A61K 9/5192; A61K 31/7052; A61K 31/7048; A61K 31/137; A61K 31/4985; A61K 47/14

(57) ABSTRACT

The present disclosure provides oleogel and oleopaste compositions, as well as methods, kits, preparations, and methods of using the same. The oleogels and oleopastes presented herein include compositions comprising azithromycin, albendazole, lumefantrine, praziquantel, moxifloxacin, and ivermectin.

### CROSS-REFERENCE TO RELATED APPLICATIONS

This application claims priority under 35 U.S.C. § 119(e) to U.S. Provisional Patent Application, U.S. Ser. No. 63/025,488 filed May 15, 2020, the contents of which are incorporated herein by reference in its entirety.

### BACKGROUND OF THE INVENTION

Dissolution and/or solubility still persists as one bottleneck for active ingredients such as pharmaceuticals, as 40% of approved drugs, and approximately 70% of drugs in development stages have poor aqueous solubility (Kalepu & Nekkanti, 2015). Biopharmaceutics classification system (BCS) & Biopharmaceutics drug disposition classification system (BDDCS) are two surrogates in in vitro and in vivo predictions of pharmacokinetics (Charalabidis et al., 2019). Even though the permeability parameter of BCS is taken over by the metabolism parameter of the new BDDCS system, dissolution parameter still plays an unchangeable role in formulation approaches to shift drug characterization into more favorable classified zones, e.g. class II into class I and class IV into class III (Pouton, 2006).

For example, children under the age of 5 are a vulnerable population that is susceptible to a host of diseases¹. There is a large disparity between children of high and low sociodemographic index countries, with the latter experiencing a much higher disease rate and resultant mortality^{2,3}. Specifically, Angola, Central Africa Republic, Chad, Mali, Nigeria, Sierra Leone and Somalia account for 20% of the world's under-5 mortality³. Hence, there remains a need to identify practical life-saving interventions and then make them available in countries that remain susceptible to high childhood mortality.

Pharmacological and nutrient-based interventions (e.g., drugs, nutraceuticals) can be used to reduce the impact of childhood diseases. Indeed, a recent study has shown that communities in which children were treated with twice-yearly broad spectrum antibiotic, azithromycin, had a reduced mortality rate in comparison to communities in which children received placebo⁴. Due to the ease and low cost of manufacturing, drugs are typically formulated as tablets. Unfortunately, children have difficulties swallowing tablets. In fact, administration of large tablets to children under age of 36 months has caused choking with fatal consequences⁵. Hence, a common practice in the field involves healthcare professionals crushing the tablet, dispersing it in water and administering the suspension to the child. Although highly attractive due to its ease, this practice introduces several points of failure⁶. First, to ensure complete absorption of the drug, the tablet needs to be dispersed in a large volume of water, which may not be easy to ingest for the patient. Second, drugs are often distasteful, making the suspension unpalatable. These two factors can lead to the child completely refusing the drug or spitting out the drug suspension. The cleanliness of water can be sub-standard, putting at risk an already debilitated child⁸. Finally, excipients used in adult formulations, although safe for adults, may be highly toxic to children⁹. Hence, dosage forms specifically designed for a young patient population can benefit global health.

Some drugs are marketed in paediatric-friendly liquid dosage forms such as solutions, syrups and suspensions. Although water has been the vehicle of choice for these liquid formulations, it is a rather imperfect carrier. Drug absorption from solutions is most rapid. However, it is challenging to formulate drugs as aqueous solutions because drugs often have limited solubility in water. This necessitates the use of co-solvents such as ethanol that are unsafe for infants¹⁰. Suspensions are an attractive alternative to solutions. However, most suspensions will require the patient, guardian or healthcare provider to mix (shake) the formulation prior to use. Failure to do so can introduce a risk of inaccurate dosing¹¹. Further, drug absorption from suspensions may be incomplete due to lack of drug solubility in physiological fluid. Hence, alternative paediatric dosage forms have long been needed. The field of paediatric drug delivery has recently been revitalized with the description of spray dried nanoparticles¹²⁻¹⁴ that are reconstituted prior to use. Due to the smaller particle size of drugs in these formulations compared to conventional suspensions, they display improved drug permeation and dissolution rate, while circumventing the need for toxic co-solvents such as ethanol. The introduction of more child-friendly solid dosage forms such as multi-particulate preparations (mini-tablets, granules, pellets) orodispersible, effervescent and chewable tablets has proven advantageous in cases of swallowing difficulties⁶. However, most of these formulations also require potable water, which may not always be freely available¹⁵. While the use of oils for delivering drugs has been widely explored, there are few studies employing oleogels as delivery systems for the oral administration of lipophilic compounds²²⁻²⁵. With multitude of ingredients available for the formation of oleogels, a systematic study understanding the influence of the various ingredients is needed.

### SUMMARY OF THE INVENTION

Described herein is a formulation strategy inspired by techniques recently described in the field of molecular gastronomy that transform oils into gels, also known as oleogels¹⁶ and oleopastes. These techniques have been used to increase the melting temperature of oils to render foods such as chocolates heat resistant¹⁷. Conversion of vegetable oils to gel-like consistency also allows for substitution of animal fats¹⁸ to satisfy dietary restrictions. Moreover, using gels instead of liquid oils prevents oil separation from foods such as cupcakes, aiding long-term storage and improving consumer satisfaction¹⁹. Plant-derived oils represent an attractive vehicle for drug delivery. Oils have a prolonged history of use in the food industry, and a very well-established safety profile. Second, oils may be more proficient at wetting or dissolving hydrophobic drugs. Finally, ingestion of fats stimulates secretion of bile salts and enzymes that enhance drug dissolution in the physiological fluids and drug absorption²⁰. Furthermore, converting oils into gels allows for altering the mouth-feel and texture of the dosage form²¹, which affects patient acceptance.

Oleoegel formulations disclosed herein (1) can be used for a variety of hydrophobic actives (2) allow for administration without large solids (3) do not require water for administration (4) are safe to use in children and (5) provide favourable pharmacokinetics.

The ability to form gels can also provide control over mouth feel and consistency of product, and can provide beneficial consumer acceptability.

An oleogel is a semisolid dosage form where gelling agents are dispersed thoroughly to build up a structured matrix which then holds any organic liquids or oils (Lupi et al., 2013). Based on the gelling agents, oleogel can be distinguished into polymer type, e.g. gelatin and xanthan gum (Patel et al., 2015), to create a crosslinked polymeric network, or into low molecular weight type, e.g. saturated fatty acids (Daniel & Rajasekharan, 2003), to induce solvent crystallization. On the other hand, in regard to processing method, oleogels can also categorized into direct dispersion, emulsion template and oil sorption (Patel & Dewettinck, 2016).

An oleopaste is also a semisolid dosage form consisting of a mixture of liquid and dispersed fine solid entrapped in the mesh of gelling agent molecules. However, due to the practically insoluble characteristic of some active ingredients (Pubchem, 2020), nanoparticles are synthesized and compounded into this oil-based system to boost aqueous drug dissolution rate.

Certain formulations disclosed herein include four components viz. drug, solubilizing agent, gelling agent and oil. Also disclosed herein are reports on the determination of the influence of inactive ingredients on formulation properties. Pharmacokinetics of representative formulations were tested for three drugs administered by the oral and rectal route in large animals. Also disclosed herein are single and multi-dose containers that can be used to easily dispense these formulations in resource-limited settings. The compositions disclosed herein address several challenges associated with formulation design and patient satisfaction, and allowing for employment in the care of a highly vulnerable, yet overlooked, patient population.

The disclosure provides oleogel and oleopaste compositions, as well as methods, kits, and preparations of the same.

In one aspect, the disclosure provides a composition comprising (a) an active ingredient, (b) an oil, (c) a gelling agent, and (d) optionally, a solubilizing agent, wherein the composition is in the form of a semisolid dosage form selected from an oleogel and an oleopaste.

In another aspect, the disclosure provides methods of treating a disease or disorder, comprising administering an effective amount of a composition of any one of the compositions described herein to a subject in need thereof.

An additional aspect provides methods of preventing a disease, comprising administering an effective amount of a composition of any one of the compositions described herein to a subject in need thereof.

In some aspects, the disclosure provides methods of delivering an active ingredient, comprising administering an effective amount of a composition any one of the compositions described herein to a subject in need thereof.

The disclosure further provides a method of overcoming the food effect of an active ingredient, comprising administering an effective amount of a composition of any one of the compositions described herein to a subject in need thereof.

In one aspect, the disclosure provides a composition as described herein made by a process comprising the steps of: mixing the oil, gelling agent, active ingredient, and optionally, solubilizing agent; heating the mixture; and cooling the mixture.

Further provided are nanoparticles as described herein made by a process comprising the steps of: dissolving the active ingredient in a first solvent system comprising an organic solvent; emulsifying the dissolved active ingredient in a second solvent system comprising a water, polymer, and a surfactant; optionally sonicating or agitating the resultant mixture; freezing the sonicated/agitated mixture; and lyophilizing the frozen mixture.

The disclosure also provides kits comprising a composition as described herein and instructions for administering the same.

The details of certain embodiments of the present disclosure are set forth in the Detailed Description of Certain Embodiments, as described below. Other features, objects, and advantages of the present disclosure will be apparent from the Definitions, Examples, and Claims.

### BRIEF DESCRIPTION OF THE DRAWINGS

FIG. 1: A non-limiting summary of drugs used with oleogel and oleopaste formuations disclosed herein.

FIG. 2: Visual central tendency of AZT concentrations impacted by heating at 90° C. after 0, 5 and 10 minutes.

FIGS. 3A-3L: Pharmacokinetic release courses of each oleogel in the screening panel.

FIG. 4: Scatterplot of AUCs from triplicates of 36 oleogel formulations as a bird's-eye view on impacts of oils, solubilizers and gelling agent.

FIGS. 5A-5F: Some pharmacokinetic parameters, i.e. average released AUC, average Agel,t=0, average AUCfit, average Krel, average Cmax and average Tmax describing each oleogel's release, absorption and elimination steps in general.

FIG. 6: Graphs comparing the 90 -minute release course between the formulation made of cottonseed oil, beeswax without solubilizer and recrystallized AZT.

FIG. 7: Graphs of mean ABZ nanoparticles size in respect to mass ratio of surfactants and polymers.

FIG. 8: Graphs of ABZ release from 3 different non-oil-based formulations, i.e. ABZ powder, ABZ physical mixture, and ABZ formulation 2.

FIG. 9: Graphs of ABZ release from oleopastes containing ABZ powder and ABZ nanoparticles synthesized from formulation 2.

FIG. 10: The serum ABZ concentration curves observed on tablet and oleopaste not using any solubilizers.

FIG. 11: Semi-natural logarithmic graphs of serum ABZ concentration curve over time observed from powder and oleopaste not using solubilizers.

FIG. 12: Graph expressing AUC of serum ABZ detected in 2 rat groups administered tablet and oleopaste without solubilizer.

FIG. 13: Graphs of serum ABZ sulfone and ABZ sulfoxide obtained from 2 groups of rats administered tablet and oleopaste without solubilizer.

FIG. 14: Graph expressing AUC of serum ABZ sulfone detected in 2 rat groups administered tablet and oleopaste without solubilizer.

FIG. 15: Graph of average solubility of ABZ powder in solubilizers, i.e. Capryol 90, Labrafac™ lipophile WL 1349 and Lauroglycol FCC, respectively.

FIG. 16: Graphs of ABZ released from different oleopastes using various soulbilizers, i.e. Capryol 90, Labrafac lipophile WL 1349 and Labrasol ALF.

FIGS. 17A-17D: Charts analyzing the WHO model list of essential medicines for children, including target diseases (FIG. 17A), various drug categories amongst the top three disease areas (FIG. 17B), popular routes of administration for the drug products (FIG. 17C), and dosage forms (FIG. 17D).

FIGS. 18A-18H: Characterization of oleogels. FIG. 18A shows gel formation in fatty acids at different concentrations. FIG. 18B shows gel strength of the oleogels formed using saturated fatty acids. FIG. 18C shows gel strength of hydroxy fatty acids, FIG. 18D shows the effect of terminal functional group on the gel strength of the oleogels. FIG. 18E shows the rheological performance of gels formed using five different waxes. FIG. 18F shows heat flow at varying temperatures. FIG. 18G shows microstructures of the gels formed using various concentrations of rice bran wax and 12-hydroxystearic acid using light microscopy. FIG. 18H shows the thermal behaviour of oleogels formed using rice bran wax with differential scanning calorimetry (DSC).

FIGS. 19A-19E: Measuring drug solubility in oil-solubilizer mixtures. FIG. 19A shows the 9 plant-based oils studied. FIG. 19B shows solubilizing agents mixed with the oils. To increase diversity. FIGS. 19C-19E show three anti-infectives used for the solubility studies, azithromycin (FIG. 19C), praziquantel (FIG. 19D) and lumefantrine (FIG. 19E).

FIGS. 20A-20C: In vitro digestion of oleogels. FIG. 20A shows an image of the digestion of oleogels in vitro in simulated salivary, gastric and intestinal conditions. FIG. 20B shows the lipolytic products generated during the digestion of the using cryo-TEM. FIG. 20C shows the amount of drug released in the various media.

FIGS. 21A-21L: Pharmacokinetics of oleogels. FIGS. 21A-21C show the pharmacokinetics of azithromycin tablets, oral and rectal oleogels. FIG. 21D shows the bioavailability of azithromycin tablets, oral and rectal oleogels. FIGS. 21E-21G show the pharmacokinetics of praziquantel. FIG. 21H shows the bioavailability (AUC) of praziquantel tablets, oral and rectal oleogels. FIGS. 21I-21K show the pharmacokinetics of lumefantrine tablets, oral and rectal oleogels. FIG. 21L shows the bioavailibility (AUC) of lumefantrine tablets, oral and rectal oleogels

FIGS. 22A-22D: Analysis of single and multi-dose containers for dispensing oleogels. FIG. 22A shows images of single and multi-dose containers for dispensing oleogels. FIG. 22B shows the amount of dose dispersed from single dose containers for three individuals. FIG. 22C shows the amount of dose dispensed from each of 4 pockets in multi-dose containers. FIG. 22D shows the amount of dose dispensed from multi-dose containers for three individuals.

FIG. 23: Graph showing the solubility of ivermectin in a subset of formulations.

FIG. 24: Chart showing the stability of ivermectin during heating. Circles are individual data points, bar is average; Ingredients: ivermectin, peceol, ricebran wax, cottonseed oil.

FIG. 25: Graph showing serum concentration for a tablet of ivermectin in pigs.

FIG. 26: Graph showing serum concentration for an oral oleogel of ivermectin in pigs.

FIG. 27: Images of four different oleopastes with the same concentration of moxifloxacin (20%) and formulated with varying amounts of cottonseed oil.

FIG. 28: Graph showing moxifloxacin concentrations in the top and bottom half of the formulation when the formulation was stored in a refrigerator (4° C.) to test it and heterogeneity.

FIG. 29: Graph showing moxifloxacin concentrations in the top and bottom half of the formulation when the formulation was stored in a refrigerator (40° C.) to test stability and heterogeneity.

FIG. 30: Graph showing moxifloxacin concentrations in the top and bottom half of the formulation when the formulation was stored at 4° C. and 60° C. to test stability and homogeneity.

FIG. 31: Graph showing pharmacokinetics of moxifloxacin formulated as an aqueous solution.

FIG. 32: Graph showing pharmacokinetics of moxifloxacin formulated as an oral oleopaste.

FIG. 33: Graph showing the solubility of albendazole in oil and oil-solubilizer formulations.

FIG. 34: Schematic showing an approach for preparing albendazole-nanoparticles via forming an emulsion of albendazole dispersed in an aqueous polymer-surfactant mixture then extraction and lyophilization.

FIGS. 35A-35D: Plots showing size of albendazole nanoparticle formulations characterized by dynamic light scattering. The size of each circle is proportional size of nanoparticles. Weight of drug=10 mg.

FIGS. 36A-36C: Graphs showing the release of albendazole either formulated as a powder (FIG. 36A), a physical mixture with a surfactant (Tween 20) and polymer (PVA) (FIG. 36B), or formulated in nanoparticles with the same polymer and surfactant (FIG. 36C). Solid line indicates average, dotted line indicates 3 repeats.

FIGS. 37A-37B: Graphs showing the rate of drug release from albendazole formulated in oleopaste either as a powder (FIG. 37A) or in the form of nanoparticles (FIG. 37B).

FIGS. 38A-38C: Graphs showing the pharmacokinetics and bioavailability of azithromycin formulations in rats. FIGS. 38A-38B show pharmacokinetics of the commercial tablets of albendazole (FIG. 38A) and oleopaste formulation (FIG. 38B). FIG. 38C shows bioavailability (AUC) of the commercial tablets of albendazole and oleopaste formulation.

### DEFINITIONS

Unless otherwise required by context, singular terms shall include pluralities, and plural terms shall include the singular.

The language “in some embodiments” and “in certain embodiments” are used interchangeably.

The following definitions are more general terms used throughout the present application:

The singular terms “a,” “an,” and “the” include plural references unless the context clearly indicates otherwise. Similarly, the word “or” is intended to include “and” unless the context clearly indicates otherwise.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

When a range of values (“range”) is listed, it is intended to encompass each value and sub-range within the range. A range is inclusive of the values at the two ends of the range unless otherwise provided.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

The term “lipophilic” or “hydrophobic” refers to the ability of a compound to dissolve, or the ability of a moiety of a compound to assist the compound in dissolving in fats, oils, lipids, and/or non-polar solvents (e.g., hexane or toluene). Lipophilic moieties include, but are not limited to, substituted or unsubstituted, branched or unbranched alkyl groups having 1 to 50 carbon atoms. In certain embodiments, the lipophilic moiety is an alkyl group including at least 1, at least 6, at least 12, at least 18, at least 24, at least 36, or at least 50 carbon atoms. In certain embodiments, the lipophilic moiety is an alkyl group including at most 50, at most 36, at most 24, at most 18, at most 12, or at most 6 carbon atoms. Combinations of the above-referenced ranges (e.g., at least about 1 and at most about 24 carbon atoms) are also within the scope of the present disclosure. In certain embodiments, the lipophilic moiety is unsubstituted alkyl. In certain embodiments, the lipophilic moiety is unsubstituted and unbranched alkyl. In certain embodiments, the lipophilic moiety is unsubstituted and unbranched C₁₋₂₄ alkyl. In certain embodiments, the lipophilic moiety is unsubstituted and unbranched C₆₋₂₄ alkyl. In certain embodiments, the lipophilic moiety is unsubstituted and unbranched C₁₂₋₂₄ alkyl.

The term “polymer” refers to a compound comprising eleven or more covalently connected repeating units. In certain embodiments, a polymer is naturally occurring. In certain embodiments, a polymer is synthetic (i.e., not naturally occurring).

The term “gel” is a nonfluid colloidal network or nonfluid polymer network that is expanded throughout its whole volume by a fluid (e.g., a solvent, such as water). A gel has a finite, usually rather small, yield stress. A gel may contain: (i) a covalent molecular network (e.g., polymer network), e.g., a network formed by crosslinking molecules (e.g., polymers) or by nonlinear polymerization; (ii) a molecular network (e.g., polymer network) formed through non-covalent aggregation of molecules (e.g., polymers), caused by complexation (e.g., coordination bond formation), electrostatic interactions, hydrophobic interactions, hydrogen bonding, van der Waals interactions, π-π stacking, or a combination thereof, that results in regions of local order acting as the network junction points. The term “thermoreversible gel” refers to a gel where the regions of local order in the gel are thermally reversible; (iii) a polymer network formed through glassy junction points, e.g., one based on block copolymers. If the junction points are thermally reversible glassy domains, the resulting swollen network may also be termed a thermoreversible gel; (iv) lamellar structures including mesophases, e.g., soap gels, phospholipids, and clays; or (v) particulate disordered structures, e.g., a flocculent precipitate usually consisting of particles with large geometrical anisotropy, such as in V₂O₅ gels and globular or fibrillar protein gels. The term “hydrogel” refers to a gel, in which the fluid is water.

The term “fluid” refers to a substance that, under a shear stress at 25° C., continually flows (e.g., at a velocity of 1 millimeter per second) along a solid boundary. Examples of fluids include liquids (e.g., solvents and solutions), gases, and suspensions (where solids are suspended in a liquid or gas). A “nonfluid” is a substance that is not a fluid.

The term “particle” refers to a small object, fragment, or piece of a substance that may be a single element, inorganic material, organic material, or mixture thereof. Examples of particles include polymeric particles, single-emulsion particles, double-emulsion particles, coacervates, liposomes, microparticles, nanoparticles, macroscopic particles, pellets, crystals, aggregates, composites, pulverized, milled or otherwise disrupted matrices, and cross-linked protein or polysaccharide particles, each of which have an average characteristic dimension of about less than about 1 mm and at least 1 nm, where the characteristic dimension, or “critical dimension,” of the particle is the smallest cross-sectional dimension of the particle. A particle may be composed of a single substance or multiple substances. In certain embodiments, the particle is not a viral particle. In other embodiments, the particle is not a liposome. In certain embodiments, the particle is not a micelle. In certain embodiments, the particle is substantially solid throughout. In certain embodiments, the particle is a nanoparticle. In certain embodiments, the particle is a microparticle.

The term “nanoparticle” refers to a particle having an average (e.g., mean) dimension (e.g., diameter) of between about 1 nanometer (nm) and about 1 micrometer (μm) (e.g., between about 1 nm and about 300 nm, between about 1 nm and about 100 nm, between about 1 nm and about 30 nm, between about 1 nm and about 10 nm, or between about 1 nm and about 3 nm), inclusive.

### DETAILED DESCRIPTION OF CERTAIN EMBODIMENTS

Before the disclosed systems, hydrogels compositions, methods, uses, and kits are described in more detail, it should be understood that the aspects described herein are not limited to specific embodiments, methods, systems, apparati, or configurations, and as such can, of course, vary. It is also to be understood that the terminology used herein is for the purpose of describing particular aspects only and, unless specifically defined herein, is not intended to be limiting.

The disclosure provides oleogel and oleopaste compositions, as well as methods, kits, preparations, and methods of using the same.

In one aspect, the disclosure provides a composition comprising (a) an active ingredient, (b) an oil, (c) a gelling agent, and (d) optionally, a solubilizing agent, wherein the composition is in the form of a semisolid dosage form selected from an oleogel and an oleopaste.

In some embodiments, the composition comprises an oil. In some embodiments, the oil is a glycerol ester or fatty acid. In some embodiments, the glycerol ester is a triglyceride or diglyceride. In some embodiments, the fatty acid is a saturated fatty acid, a monounsaturated fatty acid, an ω-3 fatty acid, an ω-6 fatty acid, a di-unsaturated fatty acid, or a mixture thereof. In some embodiments, the oil is a plant-based oil. In some embodiments, the oil is a vegetable oil. In some embodiments, the oil is canola oil. In some embodiments, the oil is vegetable oil, canola oil, flaxseed oil, lard, soybean oil, cottonseed oil, sunflower oil, peanut oil, sesame oil, olive oil, rapeseed oil, corn oil, or a mixture thereof. In some embodiments, the oil is flaxseed or cottonseed oil. In some embodiments, the oil is edible. In some embodiments, the oil acts as a solvent. In some embodiments, the oil is flaxseed oil.

The composition may comprise varying amounts of oil. In some embodiments, the composition comprises about 30% to less than 100% w/w of the oil. In some embodiments, the composition comprises about 40% to less than 100% w/w of the oil. In some embodiments, the composition comprises about 60% to less than 100% w/w of the oil. In some embodiments, the composition comprises about 75% to less than 100% w/w of the oil. In some embodiments, the composition comprises about 80% to less than 100% w/w of the oil. In some embodiments, the composition comprises about 75% to about 99.5% w/w of the oil. In some embodiments, the composition comprises about 84.5% to about 99.5% w/w of the oil. In some embodiments, the composition comprises about 75% to about 85% w/w of the oil.

In some embodiments, the composition comprises a gelling agent. In some embodiments, the gelling agent is polymer based, a wax, a fatty acid, a hydroxy acid, an unsaturated fatty acid, a saturated fatty acid, a fatty amine, a fatty alcohol, a fatty acrylate, a fatty ester, or a mixture thereof. In some embodiments, the gelling agent is polymer-based. In some embodiments, the gelling agent forms a crosslinked polymeric network in the composition. In some embodiments, the gelling agent is gelatin or xanthan gum. In some embodiments, the gelling agent is a natural wax. In some embodiments, the gelling agent is a fatty acid. In some embodiments, the gelling agent is a fatty acid selected from a hydroxy fatty acid, an unsaturated fatty acid, a saturated fatty acid, or a mixture thereof. In some embodiments, the fatty acid is lauric acid, palmitic acid, stearic acid, arachidic acid, behenic acid, or a mixture thereof. In some embodiments, the hydroxy fatty acid is 2-hydroxycaproic acid, 12-hydroxylauric acid, 3-hydroxymyristic acid, 16-hydroxypalmitic acid, 12-hyroxystearic acid, or a mixture thereof. In some embodiments, the unsaturated fatty acid is monounsaturated octadecanoic acid, elaidic acid, linoelaidic acid, oleic acid, linolenic acid, or a mixture thereof. In some embodiments, the fatty amine is stearyl amine. In some embodiments, the fatty alcohol is stearyl alcohol. In some embodiments, the fatty acrylate is stearyl methacrylate. In some embodiments, the wax is carnauba wax, candelilla wax, soy wax, rice bran wax, beeswax, castor wax, or a mixture thereof. In some embodiments, the gelling agent is stearyl alcohol, 2-hydroxycaproic acid, 12-hydroxylauric acid, 3-hydroxymyristic acid, 16-hydroxypalmitic acid, 12-hyroxystearic acid, lauric acid, palmitic acid, stearic acid, arachidic acid, behenic acid, elaidic acid, stearyl amine, rice bran wax, beeswax, castor wax, carnauba wax, candelilla wax, or a mixture thereof. In some embodiments, the gelling agent is beeswax, candelilla wax, carnauba wax, rice bran wax, stearyl alcohol, or a mixture thereof. In some embodiments, the gelling agent is beeswax, candelilla wax, carnauba wax, palmitic acid, or a mixture thereof.

The composition may comprise varying amounts of gelling agent. In some embodiments, the composition comprises greater than about 0% to about 20% w/w of the gelling agent. In some embodiments, the composition comprises greater than about 0% to about 15% w/w of the gelling agent. In some embodiments, the composition comprises about 1% to about 10% w/w of the gelling agent. In some embodiments, the composition comprises greater than about 0% to about 2% w/w of the gelling agent. In some embodiments, the composition comprises about 2% to about 4% w/w of the gelling agent. In some embodiments, the composition comprises about 9% to about 11% w/w of the gelling agent. In some embodiments, the composition comprises about 10% w/w of the gelling agent. In some embodiments, the gelling agent is C12 fatty acid, an unsaturated fatty acid, a fatty amine, or a mixture thereof and the composition comprises about 10% w/w of the gelling agent. In some embodiments, the composition comprises about 8% w/w of the gelling agent. In some embodiments, the composition comprises about 6% w/w of the gelling agent. In some embodiments, the composition comprises about 4% w/w of the gelling agent. In some embodiments, the composition comprises about 3% w/w of the gelling agent. In some embodiments, the gelling agent is a C16-C22 fatty acid and the composition comprises about 3% w/w of the gelling agent. In some embodiments, the composition comprises about 2% w/w of the gelling agent. In some embodiments, the composition comprises about 1% w/w of the gelling agent. In some embodiments, the gelling agent is a wax and the composition comprises about 1% w/w of the gelling agent.

In certain embodiments, the physical and chemical properties of the gelling agent contribute to the properties of the composition. In some embodiments, the gelling agent forms a structured matrix which then holds any organic liquids or oils in the composition. In some embodiments, the gelling agent induces solvent crystallization in the composition. In some embodiments, the gelling agent adjusts the consistency of the composition. In some embodiments, the gelling agent adjusts the viscosity of the composition. In some embodiments, the gelling agent adjusts the softening temperature of the composition. In some embodiments, the gelling agent adjusts the heat stability of the composition. In some embodiments, the gelling agent forms a dendritic microstructure in the composition. In some embodiments, the solubilizing agent increases the lipophilicity of the oil in the composition.

In some embodiments, the composition comprises a solubilizing agent. In some embodiments, the solubilizing agent is a lipophilic surfactant, a fatty acid, a fatty acid ester, or a mixture thereof. In some embodiments, the solubilizing agent is a lipophilic surfactant. In some embodiments, the solubilizing agent is a fatty acid ester of a di-alcohol or tri-alcohol. In some embodiments, the solubilizing agent is glyceryl monooleate, propylene glycol monocaprylate, glyceryl monolinoleate, polyglyceryl-3 dioleate, caprylocaproyl polyoxyl-8 glycerides, medium chain triglycerides, oleoyl polyoxyl-6 glycerides, linoleoyl polyoxyl-6 glycerides, propylene glycol monolaurate, or a mixture thereof. In some embodiments, the solubilizing agent is Peceol®, Capryol® 90, Capryol® PGMC, Maisine® CC, Plurol® Oleique CC 497, Labrasol® ALF, Labrafac™ lipophile WL 1349, Labrafil® M 1944 CS, Labrafil® M 2125 CS, Lauroglycol™ FCC, Lauroglycol™ 90, or a mixture thereof. In some embodiments, the solubilizing agent is Peceol®, Capryol® 90, ^{Maisine}® CC, or a mixture thereof. In some embodiments, the solubilizing agent is Capryol® 90 (i.e., propylene glycol esters of caprylic acid). In some embodiments, the propylene glycol esters of caprylic acid are propylene glycol monoesters and diesters of caprylic acid, or mixtures thereof. In some embodiments, the propylene glycol esters of caprylic acid are mainly propylene glycol diesters with propylene glycol monoesters of caprylic acid. In some embodiments, the solubilizing agent is a derivative of oleic acid (e.g., monoglyceride, diglyceride, triglyceride, polyethylene glycol esters (e.g., MW 300, MW 400), propylene glycol esters of oleic acid, or mixtures thereof), a derivative of caprylic acid (e.g., monoglyceride, diglyceride, triglyceride, polyethylene glycol esters (e.g., MW 300, MW 400), propylene glycol esters of caprylic acid, or mixtures thereof), a derivative of linoleic acid (e.g., monoglyceride, diglyceride, triglyceride, polyethylene glycol esters (e.g., MW 300, MW 400), propylene glycol esters of linoleic acid, or mixtures thereof), a derivative of lauric acid (e.g., monoglyceride, diglyceride, triglyceride, polyethylene glycol esters (e.g., MW 300, MW 400), propylene glycol esters of lauric acid, or mixtures thereof), a derivative of capric acid (e.g., monoglyceride, diglyceride, triglyceride, polyethylene glycol esters (e.g., MW 300, MW 400), propylene glycol esters of capric acid, or mixtures thereof), polyglyceryl-3 dioleate, polyglyceryl-3 oleate, propylene glycol monolaurate, or propylene glycol laurate, or mixtures thereof.

The composition may comprise varying amounts of solubilizing agent. In some embodiments, the composition comprises greater than about 0% to about 20% w/w of the solubilizing agent. In some embodiments, the composition comprises greater than about 0% to about 15% w/w of the solubilizing agent. In some embodiments, the composition comprises about 1% to about 10% w/w of the solubilizing agent. In some embodiments, the composition comprises greater than about 0% to about 2% w/w of the solubilizing agent. In some embodiments, the composition comprises about 2% to about 4% w/w of the solubilizing agent. In some embodiments, the composition comprises about 9% to about 11% w/w of the solubilizing agent. In some embodiments, the composition comprises about 10% w/w of the solubilizing agent. In some embodiments, the composition comprises about 8% w/w of the solubilizing agent. In some embodiments, the composition comprises about 6% w/w of the solubilizing agent. In some embodiments, the composition comprises about 4% w/w of the solubilizing agent. In some embodiments, the composition comprises about 3% w/w of the solubilizing agent. In some embodiments, the composition comprises about 2% w/w of the solubilizing agent. In some embodiments, the composition comprises about 1% w/w of the solubilizing agent. In some embodiments, the composition comprises less than 1% w/w of the solubilizing agent.

In some embodiments, composition comprises an active ingredient. In some embodiments, the active ingredient is an active pharmaceutical ingredient, a pesticide, a nutraceutical, or a cosmetic ingredient. In some embodiments, the active ingredient is an active pharmaceutical ingredient, a pesticide, or a cosmetic ingredient. In some embodiments, the active ingredient is an active pharmaceutical ingredient or a pesticide. In certain embodiments, the active ingredient is not a nutraceutical ingredient. In some embodiments, the active ingredient is not a cosmetic ingredient.

In some embodiments, the active ingredient is a pesticide. In certain embodiments, when the active ingredient is a pesticide, the composition is administered to repel insects. In certain embodiments, when the active ingredient is a pesticide, the composition is administered topically.

In some embodiments, the active ingredient is a nutraceutical ingredient. In certain embodiments, when the active ingredient is a nutraceutical ingredient, the composition is used to deliver medically or nutritionally relevant vitamins, minerals, or supplements. In certain embodiments, when the active ingredient is a nutraceutical ingredient, the composition is administered orally.

In some embodiments, the active ingredient is an cosmetic ingredient. In certain embodiments, when the active ingredient is a cosmetic ingredient, the composition is administered topically.

In some embodiments, the active ingredient is an active pharmaceutical ingredient. In some embodiments, the active pharmaceutical ingredient is a biopharmaceutics classification system class II, class III, or class IV agent. In some embodiments, the active pharmaceutical ingredient is an agent from the World Health Organization's Essential Medicines List. In some embodiments, the active pharmaceutical ingredient is an agent from the World Health Organization's Essential Medicines List for Adults. In some embodiments, the active pharmaceutical ingredient is an agent from the World Health Organization's Essential Medicines List for Children. World Health Organization's Essential Medicines List can be located at www.who.int. In some embodiments, the active pharmaceutical ingredient is an opioid analgesic, a non-opioid analgesic, an NSAID, a migraine relieving agent, an anticonvulsant, an antipsychotic, a muscle relaxant, an antidepressant, an antibacterial, an antibiotic, an antiviral, an antimalarial, an antifungal, an anthelmintic, an antiseptic, an antitrypanosomal, an antiprotozoal, an antileishmaniasis, an antiamoebic, an antigiardiasis, an multiorganismic agent, an antischistosomal, an antirematode, an antifilarial, or mixture thereof. In some embodiments, the active pharmaceutical ingredient is an antibacterial, an antibiotic, an antifungal, an antiprotozoal, or an antiviral. In some embodiments, the active pharmaceutical ingredient is stromectol, lumefantrine, roxithromycin, Abacavir, Abacavir/lamivudine, acetaminophen, Acetazolamide, Acetic acid, Acetylcysteine, Acetylsalicylic Acid, Aciclovir, Albendazole, albuterol, Allopurinol, All-trans retinoic acid, Amidotrizoate, Amikacin, Amiloride, Amiodarone, Amitriptyline, Amlodipine, Amodiaquine, Amoxicillin, Amoxicillin/clavulanic acid , amphetamine, Amphotericin B, Ampicillin, Anastrozole, Artemether, Artemether/lumefantrine, Artesunate, Artesunate/amodiaquine, Artesunate/mefloquine, Artesunate/pyronaridine, Asparaginase, Aspirin, Atazanavir, Atazanavir/ritonavir, atorvastatin, Atracurium, Atropine, Azathioprine, Azithromycin, Aztreonam, Barium sulfate, Beclometasone, Bedaquiline, Bendamustine, Benzathine benzylpenicillin, Benznidazole, Benzoyl peroxide, Benzyl benzoate, Benzylpenicillin, Betamethasone, Bevacizumab, Bicalutamide, Biperiden, Bisoprolol, Bleomycin, Budesonide, Budesonide/formoterol, Bupivacaine, Caffeine citrate, Calamine, Calcium folinate, Calcium gluconate, Capecitabine, Capreomycin, Carbamazepine, Carbidopa/levodopa , Carboplatin, Cefalexin, Cefazolin, cefdinir, Cefixime, Cefotaxime, Ceftazidime, Ceftriaxone, cephalexin, Chlorambucil, Chloramphenicol, Chlorhexidine, Chloroquine, Chloroxylenol, chlorpheniramine, Chlorpromazine, Ciclosporin, Ciprofloxacin, Cisplatin, Clarithromycin, clavulanate, Clindamycin, Clofazimine, Clomifene, Clomipramine, Clopidogrel, Clotrimazole, Cloxacillin, Clozapine, Coal tar, Codeine, Cyclizine, Cyclophosphamide, Cycloserine, Cytarabine, Dacarbazine, Daclatasvir, Dactinomycin, Dapsone, Darunavir, Dasabuvir, Dasatinib, Daunorubicin, Deferoxamine, Delamanid, Desmopres sin, Dexamethasone, dextroamphetamine, dextromethorphan, Diazepam, Diethylcarbamazine, Digoxin, Dihydroartemisinin/piperaquine, Diloxanide, Dimercaprol, diphenhydramine, Diuretics, dobutamine, Docetaxel, Docusate sodium, Dolutegravir, Dopamine, Doxorubicin, Doxycycline, Efavirenz, Efavirenz/emtricitabine/tenofovir, Efavirenz/lamivudine/tenofovir, Eflornithine, Emtricitabine/tenofovir, Enalapril, Enoxaparin, Entecavir, Ephedrine, Epinephrine , Ergometrine, Erythromycin, Erythropoiesis-stimulating agents, Estradiol cypionate/medroxyprogesterone acetate, Ethambutol, Ethambutol/isoniazid, Ethambutol/isoniazid/pyrazinamide/rifampicin , Ethambutol/isoniazid/rifampicin, Ethanol, Ethinylestradiol/levonorgestrel, Ethinylestradiol/norethisterone, Ethionamide, Ethosuximide, Etoposide, famotidine, Fentanyl, Ferrous salt, Ferrous salt/folic acid, Filgrastim, Fluconazole, Flucytosine, Fludarabine, Fludrocortisone, Fluorescein, Fluorouracil, Fluoxetine, Fluphenazine, fluticasone, fluvoxamine maleate, Folic acid, Fomepizole, Furosemide, gabapentin, Gemcitabine, Gentamicin, Gliclazide, Glucagon, Glucose, Glucose with sodium chloride, Glutaral, Glyceryl trinitrate, Griseofulvin, Haloperidol, Halothane, Heparin sodium, Hydralazine, Hydrochlorothiazide, hydrocodone, Hydrocortisone, Hydroxocobalamin, Hydroxycarbamide, Hydroxychloroquine, Hyoscine butylbromide, Hyoscine hydrobromide, Ibuprofen, Ifosfamide, Imatinib, Imipenem/cilastatin, Insulin, Intermediate-acting insulin, Iohexol, Ipratropium bromide, Irinotecan, Isoflurane, Isoniazid, Isoniazid/pyrazinamide/rifampicin, Isoniazid/pyridoxine/sulfamethoxazole/trimethoprim, Isoniazid/rifampicin , Isosorbide dinitrate, Itraconazole, Ivermectin, Kanamycin, Ketamine, Lactulose, Lamivudine , Lamivudine/nevirapine/stavudine, Lamivudine/nevirapine/zidovudine, Lamivudine/zidovudine, Lamotrigine, Latanoprost, ledipasvir/sofosbuvir, Leuprorelin, Levamisole, Levofloxacin, Levonorgestrel, Levothyroxine, Lidocaine, Lidocaine/Epinephrine, Linezolid, lisdexamfetamine, lisinopril, Lithium, Loperamide, Lopinavir/ritonavir, Loratadine, Lorazepam, Losartan, Lugol's solution, Magnesium sulfate, Mannitol, Mebendazole, Medroxyprogesterone acetate, Mefloquine, Meglumine iotroxate, Melarsoprol, Mercaptopurine, Meropenem, Mesna, Metformin, Methadone, Methotrexate, Methyldopa, methylphenidate, Methylprednisolone, Methylthioninium chloride, Metoclopramide, metoprolol, Metronidazole, Miconazole, Midazolam, Mifepristone used with misoprostol, Miltefosine, Misoprostol, mometasone, montelukast sodium, Morphine, Moxifloxacin, Mupirocin, Naloxone, Natamycin, Neostigmine, Nevirapine, Niclosamide, Nicotine replacement therapy, Nifedipine, Nifurtimox, Nilotinib, Nitrofurantoin, Norethisterone enantate, Nystatin, Ofloxacin, Ombitasvir/paritaprevir/ritonavir, Omeprazole, Ondansetron, Oseltamivir, Oxaliplatin, Oxamniquine, Oxytocin, Paclitaxel, p-aminosalicylic acid, Paracetamol, Paromomycin, Pegylated interferon-alpha-2a or pegylated interferon-alpha-2b, Penicillamine, penicillin, Pentamidine, Permethrin, Phenobarbital, Phenoxymethylpenicillin, phenylephrine, Phenytoin, Phytomenadione, Pilocarpine, Piperacillin/tazobactam, Podophyllum resin, Potassium chloride, Potassium iodide, Potassium permanganate, Povidone iodine, Praziquantel, Prednisolone, prednisolone sodium phosphate, prednisone, Primaquine, Procaine benzylpenicillin, Procarbazine, Proguanil, promethazine, Propofol, Propranolol, Propylthiouracil, Prostaglandin E, Prostaglandin El, Prostaglandin E2, Protamine sulfate, Prussian blue, Pyrantel, Pyrazinamide, Pyridostigmine, Pyrimethamine, Quinine, Raltegravir, Ranitidine, Ribavirin, Rifabutin, Rifampicin, Rifapentine, Risperidone, Ritonavir, Rituximab, Salbutamol, Salicylic acid, Selenium sulfide, Senna, sevoflurane, Silver sulfadiazine, Simeprevir, Simvastatin, Sodium calcium edetate, Sodium chloride, Sodium hydrogen carbonate, Sodium lactate, Sodium nitrite, Sodium nitroprusside, Sodium stibogluconate or meglumine antimoniate, Sodium thiosulfate, Sofosbuvir, Sofosbuvir/velpatasvir, Spectinomycin, Spironolactone, Stavudine , Streptokinase, Streptomycin, Succimer, Sulfadiazine, Sulfadoxine/pyrimethamine, sulfamethoxazole, Sulfamethoxazole/trimethoprim, Sulfasalazine, Suramin sodium, Suxamethonium, Tamoxifen, Tenofovir disoproxil fumarate , Terbinafine, Tetracaine, Tetracycline, Thioguanine, Timolol, Tranexamic acid, Trastuzumab, triamcinolone, Triclabendazole, Trimethoprim, Trimethoprim/sulfamethoxazole, Tropicamide, Tuberculin, Ulipristal, Urea, Valganciclovir, Valproic acid, Vancomycin, Vecuronium, Verapamil, Vinblastine, Vincristine, Vinorelbine, Voriconazole, Warfarin, Xylometazoline, Zidovudine , Zinc sulfate, Zoledronic acid, or a mixture thereof. In some embodiments, the active ingredient is praziquantel, azithromycin, moxifloxacin, ivermectin, lumefantrine, albendazole, or a mixture thereof.

The composition may comprise varying amounts of active ingredient. In some embodiments, the composition comprises greater than about 0% to about 60% w/w of the active ingredient. In some embodiments, the composition comprises greater than about 40% to about 60% w/w of the active ingredient. In some embodiments, the composition comprises greater than about 30% to about 60% w/w of the active ingredient. In some embodiments, the composition comprises greater than about 0% to about 30% w/w of the active ingredient. In some embodiments, the composition comprises greater than about 0% to about 10% w/w of the active ingredient. In some embodiments, the composition comprises greater than about 0% to about 1% w/w of the active ingredient. In some embodiments, the composition comprises about 6% to about 8% w/w of the active ingredient. In some embodiments, the composition comprises about 3% to about 5% w/w of the active ingredient. In some embodiments, the composition comprises about 1% to about 2% w/w of the active ingredient. In some embodiments, the composition comprises about 20% w/w of the active ingredient. In some embodiments, the composition comprises about 10% w/w of the active ingredient. In some embodiments, the composition comprises about 8% w/w of the active ingredient. In some embodiments, the composition comprises about 6% w/w of the active ingredient. In some embodiments, the composition comprises about 4% w/w of the active ingredient. In some embodiments, the composition comprises about 2% w/w of the active ingredient. In some embodiments, the composition comprises about 0.5% w/w of the active ingredient.

In certain embodiments, the active ingredient is in the form of one or more nanoparticles. In some embodiments, the nanoparticle is formed by ball milling or high pressure homogenization. In some embodiments, the nanoparticle is formed by nanoprecipitation. In certain embodiments, the nanoparticle is formed by solvent evaporation. In some embodiments, the nanoparticle has a mean size of less than about 1 μm. In some embodiments, the nanoparticle has a mean size of about 500 nm to about 1,000 nm. In some embodiments, the nanoparticle has a mean size of about 700 nm to about 850 nm. In some embodiments, the nanoparticles has a mean size of 50-400 nm. In some embodiments, the nanoparticles has a mean size of 100-200 nm.

In some embodiments, the composition comprises: azithromycin; a gelling agent selected from the group consisting of beeswax, candelilla wax, carnauba wax, and mixtures thereof; and an oil selected from the group consisting of cottonseed oil, corn oil, soybean oil, and mixtures thereof.

In certain embodiments, the composition comprises: an active ingredient; an oil; and a solubilizing agent. In certain embodiments, the composition comprises: an active ingredient; an oil selected from the group consisting of cottonseed oil, corn oil, soybean oil, flaxseed oil, and mixtures thereof; and a solubilizing agent selected from the group consisting of Peceol®, Capryol® 90, Maisine® CC, and mixtures thereof.

In certain embodiments, the composition comprises: an active ingredient; an oil; a solubilizing agent; and an antioxidant. In certain embodiments, the composition comprises: an active ingredient; an oil selected from the group consisting of cottonseed oil, corn oil, soybean oil, flaxseed oil, and mixtures thereof; a solubilizing agent selected from the group consisting of Peceol®, Capryol® 90, Maisine® CC, and mixtures thereof; and an antioxidant selected from the group consisting of propyl gallate, tertiary butylhydroquinone, butylated hydroxytoluene, butylated hydroxyanisole, a tocopherol, or mixtures thereof.

In certain embodiments, the composition comprises: an active ingredient; an oil; a gelling agent; and a solubilizing agent. In certain embodiments, the composition comprises: an active ingredient; an oil selected from the group consisting of cottonseed oil, corn oil, soybean oil, flaxseed oil, and mixtures thereof; a gelling agent selected from the group consisting of beeswax, candelilla wax, carnauba wax, rice bran wax, stearyl alcohol, palmitic acid, and mixtures thereof; and a solubilizing agent selected from the group consisting of Peceol®, Capryol® 90, Maisine® CC, and mixtures thereof.

In certain embodiments, the composition comprises: an active ingredient; an oil; a gelling agent; a solubilizing agent; and an antioxidant. In certain embodiments, the composition comprises: an active ingredient; an oil selected from the group consisting of cottonseed oil, corn oil, soybean oil, flaxseed oil, and mixtures thereof; a gelling agent selected from the group consisting of beeswax, candelilla wax, carnauba wax, rice bran wax, stearyl alcohol, palmitic acid, and mixtures thereof; a solubilizing agent selected from the group consisting of Peceol®, Capryol® 90, ^{Maisine}® CC, and mixtures thereof; and an antioxidant selected from the group consisting of propyl gallate, tertiary butylhydroquinone, butylated hydroxytoluene, butylated hydroxyanisole, a tocopherol, or mixtures thereof.

In certain embodiments, the composition comprises: an active ingredient; an oil; a gelling agent; and a solubilizing agent. In certain embodiments, the composition comprises: an active ingredient; an oil selected from the group consisting of cottonseed oil, corn oil, soybean oil, flaxseed oil, and mixtures thereof; a gelling agent selected from the group consisting of beeswax, candelilla wax, carnauba wax, and mixtures thereof; and a solubilizing agent selected from the group consisting of Peceol®, Capryol® 90, Maisine® CC, and mixtures thereof.

In certain embodiments, the composition comprises: an active ingredient; an oil; a gelling agent; a solubilizing agent; and an antioxidant. In certain embodiments, the composition comprises: an active ingredient; an oil selected from the group consisting of cottonseed oil, corn oil, soybean oil, flaxseed oil, and mixtures thereof; a gelling agent selected from the group consisting of beeswax, candelilla wax, carnauba wax, and mixtures thereof; a solubilizing agent selected from the group consisting of Peceol®, Capryol® 90, Maisine® CC, and mixtures thereof; and an antioxidant selected from the group consisting of propyl gallate, tertiary butylhydroquinone, butylated hydroxytoluene, butylated hydroxyanisole, a tocopherol, or mixtures thereof.

In certain embodiments, the composition comprises: an active ingredient; flaxseed oil; and Capryol® 90.

In certain embodiments, the composition comprises: an active ingredient; flaxseed oil; Capryol® 90; and a gelling agent.

In certain embodiments, the composition comprises: an active ingredient; an antioxidant; flaxseed oil; and Capryol® 90. In certain embodiments, the composition comprises: an active ingredient; propyl gallate; flaxseed oil; and Capryol® 90.

In certain embodiments, the composition comprises: an active ingredient; flaxseed oil; Capryol® 90; a gelling agent; and an antioxidant. In certain embodiments, the composition comprises: an active ingredient; flaxseed oil; Capryol® 90; a gelling agent; and propyl gallate.

In certain embodiments, the composition comprises: azithromycin; a gelling agent selected from the group consisting of beeswax, candelilla wax, carnauba wax, and mixtures thereof; an oil selected from the group consisting of cottonseed oil, corn oil, soybean oil, and mixtures thereof; and a solubilizing agent selected from the group consisting of Peceol®, Capryol® 90, Maisine® CC, and mixtures thereof.

In some embodiments, the composition comprises ivermectin, cottonseed oil, Peceol®, and rice bran wax.

In certain embodiments, the composition comprises praziquantel, rice bran wax, Capryol® 90, and flaxseed oil.

In some embodiments, the composition comprises azithromycin, stearyl alcohol, Capryol® 90, and flaxseed oil.

In some embodiments, the composition comprises lumefantrine, flaxseed oil and Capryol® 90.

In some embodiments, the composition comprises lumefantrine, ricebran wax, flaxseed oil and Capryol® 90.

In some embodiments, the composition comprises moxifloxacin, rice bran wax, and cottonseed oil.

In certain embodiments, the composition comprises albendazole, stearyl alcohol, and cottonseed oil.

In some embodiments, the composition comprises albendazole, stearyl alcohol, cottonseed oil and either Labrasol® ALF or Labrafac™ lipophile WL 1349.

In certain embodiments, the composition further comprises a surfactant. In some embodiments, the surfactant is sodium taurocholate hydrate, sodium oleate, Cremophor EL, Tween 20, Tween 80, or a mixture thereof. In certain embodiments, the composition further comprises a polymer. In some embodiments, the polymer is polyvinyl alcohol or hydroxypropyl methylcellulose.

In some embodiments, the composition comprises: albendazole; stearyl alcohol; cottonseed oil; hydroxypropyl methylcellulose or polyvinyl alcohol; and either Tween 20 or Cremophor EL.

In certain embodiments, the composition comprises: albendazole; stearyl alcohol; cottonseed oil; Labrasol® ALF or Labrafac™ lipophile WL 1349; polyvinyl alcohol; and Tween 20.

In some embodiments, the composition comprises: albendazole; an oil; polyvinyl alcohol; and Tween 20.

In certain embodiments, the composition further comprises an antioxidant (also known as a “stabilizer”). In some embodiments, the antioxidant improves the stability of the composition compared to the same composition without the antioxidant. In some embodiments, the antioxidant is ascorbic acid, propyl gallate, tertiary butylhydroquinone, butylated hydroxytoluene, butylated hydroxyanisole, a tocopherol, or a mixture thereof. In some embodiments, the antioxidant is butylated hydroxytoluene, butylated hydroxyanisole, tocopherol, or a mixture thereof. In some embodiments, the antioxidant is propyl gallate.

In some embodiments, the composition further comprises a flavoring agent. In certain embodiments, a provided composition further comprises a flavoring agent. In certain embodiments, the selection of a suitable flavoring agent to be added depends on the original taste sensation of the composition, including metallic, acidic, alkaline, salty, sweet, bitter and sour taste sensation. Certain flavoring agents, alone or in combination, mask specific taste sensations. For example, metallic taste could be masked with, but not limited to, flavoring agents based on berry fruits, grape, peppermint. For example, acidic taste could be masked with, but not limited to, flavoring agents based on lemon, lime, grapefruit, orange, cherry and/or strawberry. For example, alkaline taste could be masked with, but not limited to, flavoring agents based on aniseed, caramel, passion fruit, peach and/or banana. For example, salty taste could be masked with, but not limited to, flavoring agents based on butterscotch, caramel, hazelnut, spicy, maple, apricot, apple, peach, vanilla and/or wintergreen mint. For example, bitter taste could be masked with, but not limited to, flavoring agents based on licorice, passion fruit, coffee, chocolate, peppermint, grapefruit, cherry, peach, raspberry, wild cherry, walnut, mint and/or anise. For example, sweet taste could be masked with, but not limited to, flavoring agents based on grape, cream, caramel, banana, vanilla and/or fruit berry. For example, sour taste could be masked with, but not limited to, flavoring agents based on citrus flavors, licorice, root, bear and/or raspberry. Flavoring agents can be used alone or in combination and its selection will be dependent also upon the target population and any other substance (e.g., a pharmaceutical agent) incorporated on the composition. The perception of the flavoring agent changes from individual to individual and also with age: typically a geriatric population will prefer mint or orange flavors whereas younger populations tend to prefer flavors like fruit punch, raspberry, etc. Generally, the amount of flavoring agent needed to mask an unpleasant taste or improve taste overall will depend not only on the composition of the formulation but also on the flavor type and its strength. In certain embodiments, a flavoring agent is a palatable flavor that has a long shelf life and which does not crystallize or precipitate out of the composition upon storage. In certain embodiments, flavoring agents may be natural flavors, derived from various parts of the plants like leaves, fruits and flowers, or synthetic flavor oils or powders. Exemplary flavor oils that may be used in or as flavoring agents include, but are not limited to, peppermint oil, cinnamon oil, spearmint oil, and oil of nutmeg. Exemplary fruity flavors that may be used in or as flavoring agents include, but are not limited to, vanilla, cocoa, coffee, chocolate and citrus. Exemplary fruit essence flavors that may be used in or as flavoring agents include, but are not limited to, apple, raspberry, cherry, and pineapple. The amount of flavoring agent added can vary with the flavor employed. In some embodiments, the concentration of the flavoring agent in the composition is between about 0% and 5%, by weight. In some embodiments, the concentration of the flavoring agent in the composition is between 0.001% and 5%, inclusive, by weight. In some embodiments, the concentration of the flavoring agent in the composition is between 0.1% and 1%, inclusive, by weight. In some embodiments, the concentration of the flavoring agent in the composition is between 0.5% and 1%, inclusive, by weight.

In certain embodiments, the composition is administrable with limited to no water. In some embodiments, the composition is administrable with less than 50 mL, 25 mL, 10 mL, 5 mL, 2 mL, or 1 mL of water. In some embodiments, the composition is administrable without water.

In some embodiments, the composition is formulated as an oleogel or oleopaste. In certain embodiments, the composition is not an oleopaste.

In some embodiments, the composition is formulated as an oleogel. In some embodiments, the composition is formulated as an oleogel, wherein the active ingredient is praziquantel, azithromycin, ivermectin, or lumefantrine.

In some embodiments, the composition has high gel strength. In some embodiments, the composition has a G′ value of about 1×10³ to about 1×10⁶ Pa. In some embodiments, the composition has a G′ value of about 1×10⁴ to about 1×10⁶ Pa. In some embodiments, the composition has a G′ value of about 1×10⁵ Pa. In some embodiments, the G′ value is measured by TA AR2000 rheometer equipped with a 60 mm 2° cone upper geometry with a peltier stage.

In some embodiments, the composition is formulated as an oleopaste. In some embodiments, the composition is formulated as an oleopaste, wherein the active ingredient is moxifloxacin or albendazole.

In some embodiments, the composition is packaged in a plastic ampule.

In some embodiments, the composition is packaged in unit dose packaging.

In some embodiments, the composition is formulated for oral, rectal, topical, buccal, mucosal, nasal, intravaginal, intracranial, transdermal, or intraperitoneal administration. In some embodiments, the composition is formulated for oral or rectal administration. In some embodiments, the composition is formulated for oral administration. In some embodiments, the composition is formulated for rectal administration. In some embodiments, the composition is formulated for intravaginal administration.

In some embodiments, the composition provides acceptable shelf life. In some embodiments, less a percentage of the active ingredient degrades after a period of time at a temperature; wherein the percentage is about 8%, about 7%, about 6%, about 5%, about 4%, about 3%, about 2%, about 1%, or about 0.5%, the period of time is 7 days, 14 days, 28 days, 30 days, 60 days, 90 days, 120 days, 150 days, 180 days, 365 days, or 730 days, and the temperature is about 4° C., about 20° C., about 25° C., about 35° C., about 37° C., about 40° C., about 45° C., about 50° C., about 55° C., or about 60° C. In some embodiments, less than 8% of the active ingredient degrades after at least 7 days at no less than 25° C. In some embodiments, less than about 8% of the active ingredient degrades after at least 90 days at no less than about 60° C. In some embodiments, less than about 8% of the active ingredient degrades after at least 365 days at no less than about 25° C. In some embodiments, the active ingredient does not settle out of the composition. In some embodiments, less than about 10%, about 8%, about 6%, about 4%, about 3%, about 2%, or about 1% of the active ingredient settles out of the composition after about 7, about 14, about 28, about 30, about 60, about 90, about 120, about 150, about 180, about 365, or about 730 days.

In certain aspects, the oleogel or oleopaste composition has benefits over a commercially available tablet formulation of the active ingredient. In some embodiments, the composition improves the solubility of the active ingredient as compared to a commercially available tablet formulation of the active ingredient. In some embodiments, the composition improves the solubility of the active ingredient in physiological fluids as compared to a commercially available tablet formulation of the same active ingredient. In some embodiments, the composition improves the absorption of the active ingredient as compared to a commercially available tablet formulation of the same active ingredient. In some embodiments, the composition improves the bioavailability of the active ingredient as compared to a commercially available tablet formulation of the same active ingredient. In some embodiments, the AUC of the composition is the same or better as compared to a commercially available tablet formulation of the same active pharmaceutical ingredient. In some embodiments, the Cₘₐₓ of the composition is the same or better as compared to a commercially available tablet formulation of the same active pharmaceutical ingredient. In some embodiments, the composition improves the pharmacokinetics of the active pharmaceutical ingredient as compared to a commercially available tablet formulation of the same active pharmaceutical ingredient. In some embodiments, the composition improves long-term storage of the active ingredient as compared to a commercially available tablet formulation of the same active ingredient. In some embodiments, the composition exhibits no bad taste or reduced bad taste as compared to a commercially available compositions of the active ingredient.

In some embodiments, the composition provides for immediate release of the active ingredient.

In some embodiments, the composition prevents recrystallization or co-aggregation of the active ingredient in the composition.

In some embodiments, the composition does not undergo a first pass effect.

In some embodiments, the composition is in the form of an oleopaste and is extrudable from a syringe or tube using normal hand pressure.

In some embodiments, the composition is in the form of an oleopaste and the active ingredient is at least partially suspended in the composition.

In another aspect, the disclosure provides methods of treating a disease or disorder, comprising administering an effective amount of a composition of any one of the compositions described herein to a subject in need thereof. In some embodiments, the disease is a cardiovascular disease, cancer, inflammation, hormonal insufficiency, a gastrointestinal disease, malnutrition, a skin disease, poisoning, apnea, glaucoma, an infectious disease, pain, or a neurological disease. In certain embodiments, the disease is a bacterial or parasitic infection. In some embodiments, the active ingredient is used for palliative care, perioperative care, or diagnostic purposes. In certain embodiments, the composition is administered by oral administration. In certain embodiments, the composition is administered by rectal administration.

An additional aspect provides methods of preventing a disease, comprising administering an effective amount of a composition of any one of the compositions described herein to a subject in need thereof. In some embodiments, the disease is a cardiovascular disease, cancer, inflammation, hormonal insufficiency, a gastrointestinal disease, malnutrition, a skin disease, poisoning, apnea, glaucoma, an infectious disease, pain, or a neurological disease. In certain embodiments, the disease is a bacterial or parasitic infection. In some embodiments, the active ingredient is used for palliative care, perioperative care, or diagnostic purposes. In certain embodiments, the composition is administered by oral administration. In certain embodiments, the composition is administered by rectal administration.

In some aspects, the disclosure provides methods of delivering an active ingredient, comprising administering an effective amount of a composition any one of the compositions described herein to a subject in need thereof. In some embodiments, the active ingredient is an active pharmaceutical ingredient, a pesticide, a cosmetic ingredient, or a nutraceutical ingredient. In certain embodiments, the composition is administered by oral administration. In certain embodiments, the composition is administered by rectal administration.

The disclosure further provides a method of overcoming the food effect of an active ingredient, comprising administering an effective amount of a composition of any one of the compositions described herein to a subject in need thereof. In certain embodiments, the composition is administered by oral administration.

The method of any one of the preceding claims, wherein the composition improves subject compliance as compared to administration via a commercially available tablet formulation of the same active ingredient. In some embodiments, the active ingredient is an active pharmaceutical ingredient, a pesticide, a cosmetic ingredient, or a nutraceutical ingredient. In certain embodiments, the composition is administered by oral administration. In certain embodiments, the composition is administered by rectal administration.

In one aspect, the disclosure provides a composition as described herein made by a process comprising the steps of: mixing the oil, gelling agent, active ingredient, and optionally, solubilizing agent; heating the mixture; and cooling the mixture.

Further provided are nanoparticles as described herein made by a process comprising the steps of: dissolving the active ingredient in a first solvent system comprising an organic solvent; emulsifying the dissolved active ingredient in a second solvent system comprising a water, polymer, and a surfactant; optionally sonicating or agitating the resultant mixture; freezing the sonicated/agitated mixture; and lyophilizing the frozen mixture. In some embodiments, the active ingredient is albendazole. In some embodiments, the first solvent system comprises dichloromethane and acetic acid. In some embodiments, the polymer is polyvinyl alcohol or hydroxyl propyl methyl cellulose. In some embodiments, the surfactant is Cremophor EL, Tween 20, Tween 80, or a mixture thereof. In some embodiments, the aqueous to organic phase v/v ratio is about 1 to about 2. In some embodiments, the polymer to surfactant v/v ratio is about 0.5 to about 2.

The disclosure also provides kits comprising a composition as described herein and instructions for administering the same.

### Pharmaceutical Compositions, Kits, and Administration

In certain embodiments, the effective amount is a therapeutically effective amount. In certain embodiments, the effective amount is an amount effective for treating an infectious disease in a subject in need thereof. In certain embodiments, the effective amount is an amount effective for preventing an infectious disease in a subject in need thereof. In certain embodiments, the effective amount is a prophylactically effective amount. In certain embodiments, the effective amount is an amount effective for treating a proliferative disease in a subject in need thereof. In certain embodiments, the effective amount is an amount effective for preventing a proliferative disease in a subject in need thereof. In certain embodiments, the effective amount is an amount effective for treating a hematological disease in a subject in need thereof. In certain embodiments, the effective amount is an amount effective for preventing a hematological disease in a subject in need thereof. In certain embodiments, the effective amount is an amount effective for treating a neurological disease in a subject in need thereof. In certain embodiments, the effective amount is an amount effective for preventing a neurological disease in a subject in need thereof. In certain embodiments, the effective amount is an amount effective for treating a in a painful condition subject in need thereof. In certain embodiments, the effective amount is an amount effective for preventing a painful condition in a subject in need thereof. In certain embodiments, the effective amount is an amount effective for treating a psychiatric disorder in a subject in need thereof. In certain embodiments, the effective amount is an amount effective for preventing a psychiatric disorder in a subject in need thereof. In certain embodiments, the effective amount is an amount effective for treating a metabolic disorder in a subject in need thereof. In certain embodiments, the effective amount is an amount effective for preventing a metabolic disorder in a subject in need thereof. In certain embodiments, the effective amount is an amount effective for reducing the risk of developing a disease (e.g., infectious disease, proliferative disease, hematological disease, neurological disease, painful condition, psychiatric disorder, or metabolic disorder) in a subject in need thereof. In certain embodiments, the effective amount is an amount effective for inhibiting the activity (e.g., aberrant activity, such as increased activity) of an organism in a subject or cell.

In certain embodiments, the subject is an animal. The animal may be of either sex and may be at any stage of development. In certain embodiments, the subject described herein is a human. In some embodiments, the subject is an adult human. In certain embodiments, the subject is a child. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for inhibiting the activity of an organism by at least about 10%, at least about 20%, at least about 30%, at least about 40%, at least about 50%, at least about 60%, at least about 70%, at least about 80%, at least about 90%, at least about 95%, or at least about 98%. In certain embodiments, the effective amount is an amount effective for inhibiting the activity of an Src family kinase by not more than 10%, not more than 20%, not more than 30%, not more than 40%, not more than 50%, not more than 60%, not more than 70%, not more than 80%, not more than 90%, not more than 95%, or not more than 98%.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

The composition may comprise a preservative. Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include ethanol, polyethylene glycol, phenol, phenolic compounds, bisphenol, chlorobutanol, hydroxybenzoate, and phenylethyl alcohol. Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, German® 115, Germaben® II, Neolone®, Kathon®, and Euxyl®.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compositions provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compositions provided herein can be administered by any route, including enteral (e.g., oral), parenteral, intravenous, intramuscular, intra-arterial, intramedullary, intrathecal, subcutaneous, intraventricular, transdermal, interdermal, rectal, ophthalmic, intravaginal, intraperitoneal, topical, mucosal, nasal, bucal, sublingual; by intratracheal instillation, bronchial instillation, and/or inhalation; and/or as an oral spray, nasal spray, and/or aerosol. Also, contemplated routes are direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In some embodiments, the route of administration is topical (to skin, eye, ear, mouth, or affected site).

The exact amount of active ingredient required to achieve an effective amount will vary from subject to subject, depending, for example, on species, age, and general condition of a subject, severity of the side effects or disorder, identity of the particular active ingredient, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a active ingredient described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

A composition, as described herein, can be administered in combination with one or more additional pharmaceutical agents (e.g., therapeutically and/or prophylactically active agents). The compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of an organism in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects.

The composition can be administered concurrently with, prior to, or subsequent to one or more additional pharmaceutical agents, which may be useful as, e.g., combination therapies.

Also encompassed by the disclosure are kits (e.g., pharmaceutical packs). The kits provided may comprise a pharmaceutical composition described herein and a container (e.g., a vial, ampule, bottle, syringe, and/or dispenser package, or other suitable container).

In some embodiments, provided kits may optionally further include a second container comprising a pharmaceutical excipient for dilution or suspension of a pharmaceutical composition described herein. In some embodiments, the pharmaceutical composition described herein provided in the first container and the second container are combined to form one unit dosage form.

Thus, in one aspect, provided are kits including a first container comprising a composition described herein. In certain embodiments, the kits are useful for treating a disease (e.g., proliferative disease, hematological disease, neurological disease, painful condition, psychiatric disorder, or metabolic disorder) in a subject in need thereof. In certain embodiments, the kits are useful for preventing a disease (e.g., proliferative disease, hematological disease, neurological disease, painful condition, psychiatric disorder, or metabolic disorder) in a subject in need thereof. In certain embodiments, the kits are useful for reducing the risk of developing a disease (e.g., proliferative disease, hematological disease, neurological disease, painful condition, psychiatric disorder, or metabolic disorder) in a subject in need thereof. In certain embodiments, the kits are useful for inhibiting the activity (e.g., aberrant activity, such as increased activity) of an organism (e.g., a bacteria or fungus) in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, hematological disease, neurological disease, painful condition, psychiatric disorder, or metabolic disorder) in a subject in need thereof. In certain embodiments, the kits and instructions provide for preventing a disease (e.g., proliferative disease, hematological disease, neurological disease, painful condition, psychiatric disorder, or metabolic disorder) in a subject in need thereof. In certain embodiments, the kits and instructions provide for reducing the risk of developing a disease (e.g., proliferative disease, hematological disease, neurological disease, painful condition, psychiatric disorder, or metabolic disorder) in a subject in need thereof. In certain embodiments, the kits and instructions provide for inhibiting the activity (e.g., aberrant activity, such as increased activity) of an organism (e.g., a bacteria). A kit described herein may include one or more additional pharmaceutical agents described herein as a separate composition.

### EXAMPLES

In order that the present disclosure may be more fully understood, the following examples are set forth. The synthetic and biological examples described in this Application are offered to illustrate the compounds, pharmaceutical compositions, methods, and uses provided herein and are not to be construed in any way as limiting their scope.

### Section 1: Azithromycin and Albendazole

Described herein are oleogel and oleopastes. In some examples, the oleogels are semisolid dosage forms where gelling agents are dispersed thoroughly to build up a structured matrix which holds oils. In some examples, the oleopastes consists of a mixture of a liquid and dispersed fine solids, such as nanoparticles, entrapped in a mesh of gel agent molecules.

To synthesize these nanoparticles, emulsion templated freeze drying (ETFD) equipped with ultrasonication was utilized on lab scale. This means an oil-in-water emulsion was first prepared by an ultrasound probe which produces several cavity bubbles inside liquid, inducing high heat and pressure to promote nanocrystallization when these bubbles explode (Nalesso et al., 2019). Then this foam liquid was frozen under very low temperature of liquid nitrogen (Qian & Zhang, 2011). Here water molecules rapidly became tiny ice crystals, being juxtaposed with solute molecules including the nanoparticles of active ingredient and polymers. During the primary drying with low pressure, ice crystals were gradually sublimed from the frozen system, then the remaining water was desorbed during the second drying. The removal of water crystals and any solvent from the system left tiny pores inside an intertwining polymer matrix. Thanks to this porous network, nanoparticles which deposit in these pores are prevented from agglomeration into bigger size particles.

Experiments were designed using both types of low molecular weight gelling agents and directly disperse them into oils and solubilizers. In fact, natural waxes, which chemically contain diverse amounts of mostly hydrocarbons, free fatty acids, free fatty alcohols and wax esters (Doan et al., 2017), were chosen because they undergo gelation at concentration lower than 10% (Pehlivanoglu et al., 2018). Meanwhile, sole 1-stearyl alcohol was compounded in an oleopaste because of its advantage of gelation at a lower concentration than the stearic acid. Also, oils are naturally chemical combination of glycerol esters, namely triglycerides and diglycerides, and a wide spectrum of fatty acids with varying chain length (usually C₁₂C₂₂), i.e. saturated acids, monounsaturated acids, ω-3 and ω-6 acids (Hamm & Hamilton, 2013). Likewise, solubilizers deployed in experiments included lipophilic surfactants, for example, Peceol (glyceryl mono-oleate, HLB 1) (Gattefosse, 2020), Capryol 90 (propylene glycol monocaprylate, HLB 6), Maisine CC (glyceryl monolinoleate, HLB 1) , Plurol Oleique CC 497 (polyglyceryl-3 dioleate, HLB 3), Labrasol®ALF (caprylocaproyl polyoxyl-8 glycerides, HLB 12), Labrafac™ lipophile WL 1349 (medium chain triglycerides, HLB 1), Labrafil® M 1944 CS (oleoyl polyoxyl-6 glycerides, HLB 9), Labrafil® M 2125 CS (linoleoyl polyoxyl-6 glycerides, HLB 9) and Lauroglycol FCC (propylene glycol monolaurate, HLB 5).

In fact, the lipid-based system offers many advantages. For examples, oleogel can be employed as a delivery system with designed release rate of active ingredients, or as a protection means against recrystallization or coaggregation, or even help to boost the solubility and even bioavailability of drugs (O'Sullivan et al., 2016). Also, it employs edible oils which are generally recognized as safe (GRAS), increasing its credibility in terms of pharmaceutical and natural grade of excipient source. Last but not least, due to its soft consistency, patient compliance is improved in some patient groups having difficulty in swallowing, especially in children (Kernell et al., 2018).

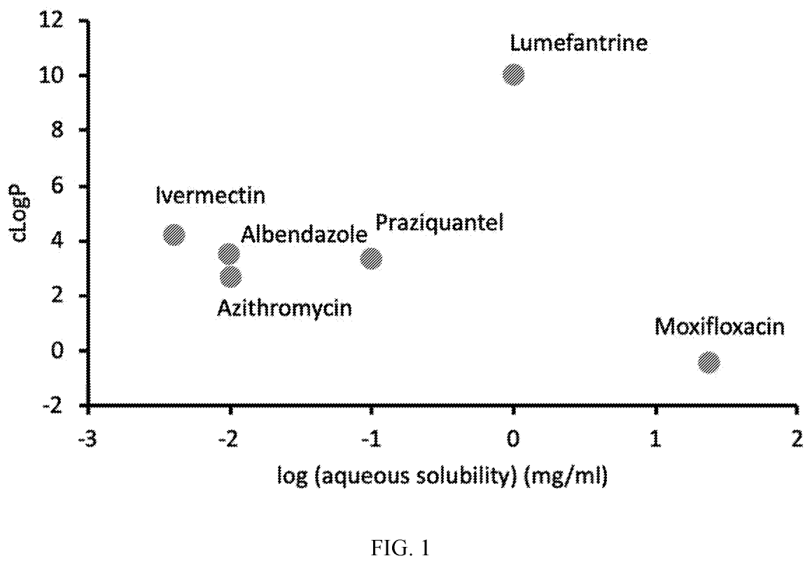

The mentioned benefits from this lipid-based system come from the lipid biochemistry in the body where exogenous and endogenous pathway concurrently exist. There drug solubilized in lipid vehicle can also be hitchhiked. Physiologically, lipid-based formulations are mainly studied at the intestinal level as the lipid digestion usually gets started there. Specifically, the formulation comprises a lipid mixture which is dispersed in the gastrointestinal lumen into smaller lipid vesicles or micelles in the presence of bile salts as a natural surfactants secreted there (Porter & Charman, 1997). Lipases transform each triglyceride from oils into 2-monoglyceride and 2 molecules of fatty acids while bile salts micellize and help to pack drug and these fatty components to eventually get absorbed into the lacteal lymphatic system. Thus, taking the lipophilic property of a prodrug to increase bioavailability as a good example, oleogel and oleopaste are promising to provide a shortcut to bypass the hepatic first pass system, which is meaningful to those drugs extensively metabolized there. Also, the surfactant as an excipient is believed to increase the drug permeability of intestinal membrane by loosening tight junctions, facilitating the paracellular transportation (Mine & Zhang, 2003). A non-limiting summary of drugs used with oleogel and oleopastes is shown in FIG. 1.

Here azithromycin (AZT) was studied as a model in certain disclosed oleogel experiments. Interestingly, AZT is broadly classified as a BCS class II/III/IV agent, bearing its low or high solubility or/and low permeability (Kauss et al., 2013) (Stieger et al., 2017). This cross categorization may reflect some limitations in scientific understanding about AZT even though it was approved by FDA in 1991 (Omudhome Ogbru, 2020). Additionally, AZT is known to be easily degraded in low pH of stomach when it is in contact with gastric juice, so fed state is likely to counteract the AZT stability by prolonging the gastric retention time of both food and administered formulation (Curatolo et al., 2011). The AZT absorption seems dosage—form dependent when co-administered with food, as food can help exert positive or negative effects on AZT pharmacokinetics (Food and Drug Administration, 2011). Also, because AZT is known to have good tissue distribution and intracellularly accumulation, it is widely administered to treat respiratory tract infections and soft tissue infections (Matzneller et al., 2013). Its intracellular accumulation is largely affected by P-gp molecules, given that both suboptimal intracellular concentration in multidrug-resistant cells by P-gp expression (Nichterlein et al., 1998), and AZT biliary and intestinal clearance mediated by P-gp and Mrp2 in rats are observed (Sugie et al., 2004).

Albendazole (ABZ) was also formulated using this strategy due to its very low water solubility (Koradia & Parikh, 2012). Thus, the absorption extent of this BCS class II or class IV agent is also very limited (<5%) (Lindenberg et al., 2004), leading to demands to optimize the stringent dissolution profile of ABZ (Press, 2010). Indeed, according to Medscape, ABZ is clinically used to eliminate a variety of intestinal worms but requires continuous mass dosing to treat systemic parasite conditions. In order to overcome this permeability obstacle, ABZ dissolution is supposed to be enhanced dramatically by using nanocrystals thanks to its promisingly increased solubility and dissolution rate (Gigliobianco et al., 2018). In addition to employing drug nanocrystals, lipid-based system also promises improved bioavailability of ABZ as this agent is better absorbed in fed state (Romo et al., 2014). As a result, an oleopaste ABZ nanocrystal may bring a pharmacokinetic leap in terms of drug solubility and absorption extent.

As a wide-spectrum antihelminth, ABZ can clinically treat infections of both intestinal and systemic parasitic worms and its related larvae when ABZ gets into the mesenteric(Medscape, 2020). In other words, ABZ which remains inside the intestine eliminates the intestinal parasites while ABZ and its active metabolite, namely albendazole sulfoxide (ABZSO), are transported to other organs. Specifically, in both rats and human, ABZ is biostransformed to active ABZSO metabolite by CYP 3A4 enzymes and flavin containing monooxygenases (FMO) while ABZSO is further oxidized to the inactive form albendazole sulfone (ABZSO2) by CYP 1A enzymes (Velík et al., 2003). The metabolism of ABZ into its active form happens at both intestinal and hepatic levels (Villaverde et al., 1995) (Rawden et al., 2000). Unlike some drugs being simultaneously a CYP 3A4 substrate and a P-gp inhibitor (Kim et al., 1999), ABZ is proved not to be effluxed by P-gp (Merino et al., 2002).

### Methods & Materials

AZT and ABZ were purchased from Tokyo Chemical Industry. Roxithromycin was obtained from Alfa Aesar. Sodium taurodeoxycholate hydrdate was purchased from Biosynth international Inc. Beeswax, carnauba wax, and candelilla wax were purchased from Stakich Inc., Luxuriant, and Plant Guru respectively. Peceol, Capryol 90, Maisine CC, Plurol Oleique CC 497, Labrasol®ALF, Labrafac™ lipophile WL 1349, Labrafil® M 1944 CS, Labrafil® M 2125 CS and Lauroglycol FCC were gifts from Gattefossé. All other chemicals were obtained from Sigma Aldrich.

### Synthesis of AZT Oleogels

Oleogel was produced mixing AZT, oils, gelling agent with or without solubilizer. Thirty-six combinations of AZT oleogels were prepared for studying the effect of each component on drug release. The components used for forming the gel are shown below:

TABLE A

| Solubilizers | Oils | Gelling agents |
|---|---|---|
| None | Cottonseed oil | Beeswax |
| Peceol | Corn oil | Candelilla wax |
| Maisine CC | Soybean oil | Carnauba wax |
| Caproyl 90 | | |

The formulation composition is listed below:

TABLE B

| Components | Weight Percentage (% w/w) | Amount (mg) |
|---|---|---|
| AZT | 0.4 | 40.0 |
| Solubilizer | 10.0 | 1 000.0 |
| Gelling agent | 5.0 | 500.0 |
| Oil | q.s. | 8 460.0 |

A slight modification of oil amount was adjusted in formulations without solubilizer

TABLE C

| Components | Weight Percentage (% w/w) | Amount (mg) |
|---|---|---|
| AZT | 0.4 | 40.0 |
| Gelling agent | 5.0 | 500.0 |
| Oil | q.s. | 9 460.0 |

To prepare the formulations, AZT, oil and solubilizer (if used) were mixed in a 20-ml glass vial and sonicated in a water bath for 60 minutes until the liquid became clear. Then gelling agent was added into the liquid while being stirred on heating plate with stir bar at 90° C. for 5 minutes. Once the gelling agent melted, the stir bar was removed, and the formulations were cooled to room temperature to form the oleogels (Table 1.1).

TABLE 1.1
Screening panel of AZT formulations.

| Formulations | Solubilizers | Oils | Waxes |
|---|---|---|---|
| 1 | Peceol | Cottonseed oil | Beeswax |
| 2 | Peceol | Cottonseed oil | Carnauba wax |
| 3 | Peceol | Cottonseed oil | Candelilla wax |
| 4 | Peceol | Soybean oil | Beeswax |
| 5 | Peceol | Soybean oil | Carnauba wax |
| 6 | Peceol | Soybean oil | Candelilla wax |
| 7 | Peceol | Corn oil | Beeswax |
| 8 | Peceol | Corn oil | Carnauba wax |
| 9 | Peceol | Corn oil | Candelilla wax |
| 10 | Maisine | Cottonseed oil | Beeswax |
| 11 | Maisine | Cottonseed oil | Carnauba wax |
| 12 | Maisine | Cottonseed oil | Candelilla wax |
| 13 | Maisine | Soybean oil | Beeswax |
| 14 | Maisine | Soybean oil | Carnauba wax |
| 15 | Maisine | Soybean oil | Candelilla wax |
| 16 | Maisine | Corn oil | Beeswax |
| 17 | Maisine | Corn oil | Carnauba wax |
| 18 | Maisine | Corn oil | Candelilla wax |
| 19 | Caproyl 90 | Cottonseed oil | Beeswax |
| 20 | Caproyl 90 | Cottonseed oil | Carnauba wax |
| 21 | Caproyl 90 | Cottonseed oil | Candelilla wax |
| 22 | Caproyl 90 | Soybean oil | Beeswax |
| 23 | Caproyl 90 | Soybean oil | Carnauba wax |
| 24 | Caproyl 90 | Soybean oil | Candelilla wax |
| 25 | Caproyl 90 | Corn oil | Beeswax |
| 26 | Caproyl 90 | Corn oil | Carnauba wax |
| 27 | Caproyl 90 | Corn oil | Candelilla wax |
| 28 | None | Cottonseed oil | Beeswax |
| 29 | None | Cottonseed oil | Carnauba wax |
| 30 | None | Cottonseed oil | Candelilla wax |
| 31 | None | Soybean oil | Beeswax |
| 32 | None | Soybean oil | Carnauba wax |
| 33 | None | Soybean oil | Candelilla wax |
| 34 | None | Corn oil | Beeswax |
| 35 | None | Corn oil | Carnauba wax |
| 36 | None | Corn oil | Candelilla wax |

### Measurement of Stability of AZT During Synthesis of Oleogel

Twenty-five milligrams of AZT were added to cottonseed oil in a glass vial at concentration of 1 mg/g. After 60 minutes of sonication, all drug powder was dissolved. The AZT solution was divided into three parts (8 ml each). One part was maintained at room temperature, while the other two were placed on a hot plate at 90° C. for 5 minutes and 10 minutes. Forty microliters from each vial were pipetted into 1.5-ml microcentrifuge tube containing 1 ml of methanol. AZT was extracted from the oil phase into the methanol phase by shaking on a horizontal shaker overnight. Following overnight extraction, the oil phase was separated from the methanol phase by centrifugation at 14000 RPM for 10 minutes. The methanol phase was diluted before analysis using liquid chromatography-mass spectrometry (LC-MS).

LC-MS Analysis of AZT

AZT concentration in methanol extract was analysed by LC-MS on Agilent 1260 Infinity I Quaternary LC equipped with a diode array detector (DAD) and Agilent 6120B Single Quadrupole mass spectrometer hardware and software. Roxithromycin (RXT) was used as an internal standard of AZT analysis. The linear range of AZT detection was 10-5000 ng/ml. Isocratic elution was set up at 20nM ammonium acetate in water:acetonitrile:methanol=20:20:60 with injection volume of 5 μl. Zorbax Eclipse XDB C18 column (4.6×150 mm, 5 μm) was employed at 50° C. The flow rate was 0.75 ml/min while chromatograms were recorded at wavelength of 210 nm, bandwidth of 4.0 nm and 5 Hz data acquisition rate.

Agilent 6120B Single Quadrupole hardware connected with a liquid nitrogen tank as a source of flow spray for atomization or nebulization. It was set up at 350° C. of gas temperature, 35 psi of nebulizer pressure and 101/min of drying gas flow rate. Also, quadrupole was heated up to 100° C. SIM selective ion monitoring mode was selected from the time programming. AZT peak appeared at retention time of 3.4 mins with the mass over charge ratio of 749.6 (m/z). Likewise, RXT was eluted at retention time of 3.9 mins with the mass over charge ratio of 837.6 (m/z). The number of fragments was 70. The dwell time was 290 ms with 100% of percentage dwell time between AZT and RXT.

Measuring Drug Release from AZT Oleogels

Extensive in vitro release studies were conducted to understand the impact of oil, solubilizer and gelling agent on the release of AZT in simulated intestinal fluid (SIF).

Preparation of lipolysis buffer: To prepare lipolysis buffer, tris-maleate (0.474 g), calcium chloride dihydrate (0.275 g), sodium chloride (8.766 g) were weighed and distilled water was added to bring the volume to 1 L. The pH of the buffer was adjusted to pH 6.5 using 5N sodium hydroxide.

Preparation of lipolysis media: L—a phosphotidylcholine (0.576 g/l) and sodium taurodeoxycholate hydrate (1.619 g/l) were added to the lipolysis buffer. Next, the solution was stirred overnight to dissolve all ingredients.

Preparation of pancreatin solution: Three grams of pancreatin was added to 15 ml of lipolysis buffer and stirred on a magnetic stir plate for 10 minutes. The mixture was centrifuged at 28000g, at 5° C. for 10 minutes. The supernant was collected and the pH of the supernant was adjusted to pH 6.5 using 5N sodium hydroxide solution.

Preparation of negative control: To avoid the technical difficulty in material weighing, 40 mg of AZT powder was dispersed into 10 ml of methanol. It was sonicated for 1 minute to get a clear solution. One milliliter was taken into 3 separate Falcon tube and remained still to evaporate the solvent.

Release studies: To analyse drug release from the formulation, 1 g of each formulation was weighed in 50 ml Falcon tubes, and 36 ml of pre-warmed lipolysis medium was added. The formulation was dispersed in the lipolysis medium by placing the tubes on an incubator shaker at 37° C. and 200 RPM for 10 minutes. To these dispersed samples, 4 ml of pancreatin solution was added, which marked the beginning of the release study. The tubes were placed on an incubator shaker at 37° C. and 200 RPM throughout the study. At various times, aliquots (1 ml) were collected and the action of pancreatin was ceased by addition of 5 μl of 1M 4-bromophenylboronic acid in methanol. These tubes were then centrifuged at 21000×g for 30 minutes. Supernatant from each tube was collected and diluted before LC-MS analysis.

### Pharmacokinetic Modeling

In vivo pharmacokinetics of the various formulations were calculated using the data obtained from in vitro release studies. To achieve this, pharmacokinetic modeling studies were conducted in two steps. In the first step, first order equations to in vitro release data were used to estimate rate constant for drug release (kᵣₑₗ) and the extent of drug release (A_{gel, t=0}). The rate constant of drug release was considered a measure of the speed of drug release. The extent of drug release or A_{gel, t=0} was a measure of the total amount of drug that could be released from the formulation at 90 min, which likely depended on the partition coefficient for AZT between the oily formulation and aqueous release medium. Hence, although the same amount of drug was added in each formulation, the extent of drug release differed across formulations. In the second step, one-compartment oral drug administration model was constructed. Using kᵣₑₗ and A_{gel, t=0} as input, pharmacokinetic parameters such as maximum concentration (Cₘₐₓ), time to maximum concentration (Tₘₐₓ) and serum drug exposure or area under the curve (AUC_{fit}) were determined.

### Step 1: Fitting First Order Release Curve to In Vitro Release Data:

To model the release of drug from the formulation, a model as described below:

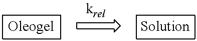

The various parameters used to describe this model are shown below:
the amount of AZT in oleogel in formulation at any time t=0 was called A_{gel, t=0} in mg.
the amount of AZT in lipolysis media at the beginning was called A_{sol, t=0} in mg.
the amount of AZT in oleogel in formulation at any time t was called Aₛₒₗ in mg.
the release rate constant from oleogels into lipolysis medium was called kᵣₑₗ in h⁻¹.

Pharmacokinetic modelling in the release step of oleogels in lipolysis media were studied with these assumptions:
at t=0, Aₛₒₗ is equal to 0 mg
the in vitro release system composes of 2 compartments
the release pharmacokinetic of oleogels follows 1^{st} order kinetic based on observation of release courses in form of a curve rather than a constant line. The mathematical equation describing the release rate of AZT from oleogels as below

$$\frac{{dA}_{gel}}{dt} = - {K}_{rel} {A}_{gel} \qquad ( 1 )$$

the in vitro release system consisting of 2 compartments was closed as all the released drug from oleogel stay in lipolysis media. Hence, it was mathematically constructed as below

$$\frac{{dA}_{sol}}{dt} = {k}_{rel} {A}_{sol} \qquad ( 2 )$$

$$\frac{dAgel}{dt} + \frac{dAsol}{dt} = 0 \qquad ( 3 )$$

Which means eventually there was no change in actual drug amount in system and no exchange with surroundings, i.e. closed system, even though the rate of release from oleogel and rate of deposition in media in respect to time may fluctuate. This held true when a summation of equation 1 and 2 was made and matched with equation 3 stated.

To calculate the A_{gel} at any time, a finite integral from 0 to t was taken on both sides of equation 1 after rearrangement:

$$⇔ \frac{{dA}_{gel}}{{A}_{gel}} = - {k}_{rel} dt \qquad ( 1 )$$

$$⇔ {{\int }_{0}}^{t} \frac{{dA}_{gel}}{{A}_{gel}} = - {{\int }_{0}}^{t} {k}_{rel} dt$$

$$⇔ ln {{A}_{gel}}_{0}^{t} = - {k}_{rel} {t}_{0}^{t}$$

$$⇔ ln {A}_{gel} - ln {A}_{gel , t = 0} = - {k}_{rel} t$$

$$⇔ ln \frac{{A}_{gel}}{{A}_{gel , t = 0}} = - {k}_{rel} t$$

$$⇔ \frac{{A}_{gel}}{{A}_{gel , t = 0}} = {e}^{- {k}_{rel} t}$$

$$⇔ {A}_{gel} = {A}_{gel , t = 0} {e}^{- {k}_{rel} t}$$

Moreover, from equation 1, 2 and 3, the mathematical relationship between the rate of release from oleogel and rate of deposition in media in respect to time could be concluded

$$\frac{{dA}_{sol}}{dt} = - \frac{{dA}_{gel}}{dt} = {k}_{rel} {A}_{gel}$$

Equation 4 was substituted into the equation above

$$⇔ \frac{{dA}_{sol}}{dt} = {k}_{rel} {A}_{gel , t = 0} {e}^{- {k}_{rel} t} \qquad ( 5 )$$

$$⇔ {dA}_{sol} = {k}_{rel} {A}_{gel , t = 0} {e}^{- {k}_{rel} t} dt$$

$$⇔ {{\int }_{0}}^{t} {dA}_{sol} = {{\int }_{0}}^{t} {k}_{rel} {A}_{gel , t = 0} {e}^{- {k}_{rel} t} dt$$

$$⇔ {{A}_{sol}}_{0}^{t} = {k}_{rel} {A}_{gel , t = 0} ( \frac{{{e}^{- {k}_{rel} t}}_{0}^{t}}{- {k}_{rel}} )$$

$$⇔ {A}_{sol} - 0 = - {A}_{gel , t = 0} ( {e}^{- {k}_{rel} t} - {e}^{0} )$$

$$⇔ {A}_{sol} = {A}_{gel , t = 0} ( 1 - {e}^{- {k}_{rel} t} )$$

This equation was fitted to the obtained in vitro data to obtain values for A_{gel, t=0} and krel.

### Step 2: Predicting In Vivo Pharmacokinetics

To model in vivo pharmacokinetics using the obtained in vitro release data, an oral absorption model was conducted as shown below:

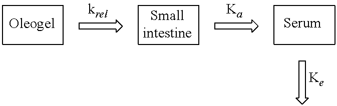

The various parameters used to describe this model are shown below:
the amount of AZT in oleogel in formulation at the beginning was called A_{gel,t=0} in mg.
the amount of AZT in oleogel in formulation at any time t was called A_{gel} in mg.
the amount of AZT in small intestine at any time t was called A_{git} in mg.
the amount of AZT in serum at any time t was called A in mg.
the release rate constant from oleogels into lipolysis medium was called kᵣₑₗ in h⁻¹.
the absorption rate constant of released AZT amount in gut permeated into serum was called kₐ in h⁻¹.
the elimination rate constant of serum AZT metabolized or eliminated was called kₑ in h⁻¹.
the drug exposure of serum AZT was called AUC_{fit} in mg·h/l.
the maximal concentration of serum AZT was called Cₘₐₓ in mg/l.
the time where serum AZT reaches Cₘₐₓ was called Tₘₐₓ in h.

Pharmacokinetic modelling in the release step of oleogels in lipolysis media were studied with these assumptions:
at t=0, A_{git} was equal to 0 mg and A was either to 0 mg
the in vivo modelling employed a multicompartmental model
The pharmacokinetic system was open.
All drug release, absorption and elimination steps followed 1st kinetic order.

$$\frac{{dA}_{gel}}{dt} = - {k}_{rel} {A}_{gel} \qquad ( 6 )$$

$$\frac{dAgit}{dt} = {k}_{rel} {A}_{gel} - {k}_{a} {A}_{git} \qquad ( 7 )$$

$$\frac{d A}{dt} = {k}_{a} {A}_{git} - {k}_{e} A \qquad ( 8 )$$

Integrations of these ordinary differential equations with respect to t were taken by R programming with packages gplots, mosaic, mosaicCalc and dplyr. Additionally, all constants were provided when kᵣₑi was deduced from Excel calculations from the previous in vitro release modelling and kₐ=0.452 h⁻¹ (Beringer et al., 2005). Meanwhile, kₑ were determined from volume of distribution V_{d} (31.1 1/kg) and clearance (Cl=630 ml/min) of Zithromax tablet (Food and Drug Administration, 2011) by

Cl=kₑV_{d}

Assuming that a normal, healthy adult weighed averagely 70 kg, then

$$⇔ {k}_{e} = \frac{0.63 l / min \text{.60}}{{V}_{d}} = 0.0 \; 17 {h}^{- 1}$$

$$where$$

$${V}_{d} = 31.1 \frac{1}{kg} \text{.70} kg = 2177 l$$

$$Also ,$$

$${T}_{1 / 2} = \frac{ln 2}{{k}_{e}} \approx 40.77 h$$

Assuming that AZT was eliminated almost completely from the body at the time point which was 6 times of T_{1/2}, the definite integral of the serum AZT amount curve over time from 0 to 250 h was also equal to the integral from 0 to ∞, and the AUC_{fit} was equal to this integral estimation number over the V_{d} as V_{d} is a constant at all time. Like the AUCfit, the Cmax was programmed to be calculated from the highest value of A divided by V_{d}, and the Tₘₐₓ were determined at the time point where the Cₘₐₓ was.

### Synthesis and Characterization of Nanoparticle Formulations of ABZ

Unlike AZT, ABZ did not dissolve in oil formulations. Hence, a suspension-based formulation of ABZ was produced. To maximize the rate of drug release from these formulations, the production of nanometer-sized particles was investigated. To achieve this, ABZ was dissolved in a mixture of dichloromethane and acetic acid (95:5 v/v) at a concentration of 20 mg/ml. The resultant non-polar phase was emulsified in an aqueous phase consisting of a polymer and surfactant. The emulsion was sonicated using a probe sonicator on an ice bath. The nano-emulsion was then frozen in liquid nitrogen and lyophilized. Nanoparticles so prepared were characterized for their size using dynamic light scattering (DLS).

Two polymers [poly(viny alcohol) (PVA) and hydroxypropyl methyl cellulose (HPMC)] and two surfactants (Cremophor EL and Tween 20) were used in different ratios to form a panel of formulations (Table 1.1). In these formulations, solutions of PVA and HPMC in water were prepared to achieve the concentrations of 20 and 5 mg/ml, respectively. Tween 20 and Cremophor EL were dissolved in water to obtain the aqueous solutions of 10 mg/ml.

TABLE 1.2
Screening panel of ABZ nanoparticle formulations.

| | | | Aqueous:Organic | |
|---|---|---|---|---|
| | | | phase | Polymer:Surfactant |
| Formulation | Polymer | Surfactant | (v:v ratios) | (v:v ratios) |
| 1 | PVA | Tween 20 | 1 | 0.5 |
| 2 | PVA | Tween 20 | 1 | 1 |
| 3 | PVA | Tween 20 | 1 | 2 |
| 4 | PVA | Tween 20 | 2 | 0.5 |
| 5 | PVA | Tween 20 | 2 | 1 |
| 6 | PVA | Tween 20 | 2 | 2 |
| 7 | PVA | Cremophor EL | 1 | 0.5 |
| 8 | PVA | Cremophor EL | 1 | 1 |
| 9 | PVA | Cremophor EL | 1 | 2 |
| 10 | PVA | Cremophor EL | 2 | 0.5 |
| 11 | PVA | Cremophor EL | 2 | 1 |
| 12 | PVA | Cremophor EL | 2 | 2 |
| 13 | HPMC | Tween 20 | 1 | 0.5 |
| 14 | HPMC | Tween 20 | 1 | 1 |
| 15 | HPMC | Tween 20 | 1 | 2 |
| 16 | HPMC | Tween 20 | 2 | 0.5 |
| 17 | HPMC | Tween 20 | 2 | 1 |
| 18 | HPMC | Tween 20 | 2 | 2 |
| 19 | HPMC | Cremophor EL | 1 | 0.5 |
| 20 | HPMC | Cremophor EL | 1 | 1 |
| 21 | HPMC | Cremophor EL | 1 | 2 |
| 22 | HPMC | Cremophor EL | 2 | 0.5 |
| 23 | HPMC | Cremophor EL | 2 | 1 |
| 24 | HPMC | Cremophor EL | 2 | 2 |

Based on the results of particle size experiments, one formulation (formulation 2 in Table 1.2) was selected for further testing. For its scaled up formulation, 200 mg of ABZ powder was dissolved in 10 ml mixture of dichloromethane : glacial acetic acid (95:5 in volume). Ten milliliters of polymer-surfactant solution was prepared by mixing 5 ml of 20 mg/ml PVA and 5 ml of 10 mg/ml Tween 20 in a 50 ml Falcon tube. To this polymer-surfactant solution, ABZ solution was added gradually on the wall of Falcon tube. The mixture was homogenized (Silverson Homogenizer) at speed of 8000 rpm for 3 minutes and transferred into a 50-ml round bottom flask immediately. Then, the flask was dipped into liquid nitrogen bath for 5 minutes to freeze the mixture. The organic solvent was evaporated using a rotovap (Buchi R-215 Rotavapor System) for 20 minutes at <20 mbar, 4° C. with an ice bath. Following the 20-minute evaporation, the freezing and evaporation step were repeated twice to ensure maximal removal of organic solvent. Finally, the flask was placed at −80 C for 1 h and then on a lyophilizer for 2 days to remove solvents.

### Effect of Nanosizing on Drug Release of ABZ

To test the effect of nanosizing, the rate of release of ABZ in SIF when ABZ was used unformulated and when formulated in nanoparticles was compared. Additionally, a physical mixture of ABZ, PVA and Tween 20 was prepared to delineate the effects that the excipients had on drug release and the effect of nanosizing.

For the release study, 17.5 mg of the nanoparticle formulation, or physical mixture were placed in a 50-ml falcon tube. ABZ powder equivalent to the amount present in the physical mixture and nanoparticle formulation was used for the drug release. SIF was added to each formulation and drug release studies were conducted as described for AZT. Concentration of ABZ in release media was measured using HPLC.

### HPLC Method for Analyzing ABZ

ABZ samples after being prepared with methanol were analysed by Agilent 1260 Infinity I Quaternary LC equipped with a diode array detector (DAD). Isocratic elution of 0.1% formic acid in distilled water and acetonitrile was 55:45 with flow rate of 1 ml/min. Injection volume was 5 μl, and Zobrax Eclipse XDB C18 column (4.6×150 mm) was used at 40° C. The running time was 6 minutes and the retention time of ABZ is 3.4 mins. DAD was employed, and readout was detected at the wavelength of 254 nm, bandwidth of 4.0 nm and 10 Hz data acquisition rate. The method gained good peak shape and symmetry.

Synthesis of ABZ Oleopaste without Solubilizers

An oleopaste used ABZ nanoparticles and another one used ABZ powder as a reference were formulated with an oil, a gelling agent and no solubilizers to test if there was any favorable effect of nanosizing observed in an oil-based system.

Oleopaste containing ABZ powder is prepared according to the below table:

TABLE D

| Ingredients | % w/w | Amount (mg) | |
|---|---|---|---|
| ABZ | 4.0 | 60.0 | mg |
| 1-stearyl alcohol | 6.0 | 90.0 | mg |
| Cottonseed oil | q.s. | 1350.0 | mg |

Accordingly, 1-stearyl alcohol and cottonseed oil were weighed into glass vial. Then this glass vial containing a stir bar was placed on a heating plate at 70° C. and 1000 rpm to melt the stearyl alcohol. Next, ABZ powder was added into this hot melt liquid and taken off from this heating plate after being evenly dispersed in hot oily vehicle.

Oleopaste containing ABZ nanoparticles from formulation 2 with 50% w/w is prepared according to the below table:

TABLE E

| Ingredients | % w/w | Amount (mg) | |
|---|---|---|---|
| ABZ nanoparticles | 8.0 | 120.0 | mg |
| 1-stearyl alcohol | 6.0 | 90.0 | mg |
| Cottonseed oil | q.s. | 1290.0 | mg |

ABZ nanoparticles were weighed into vials. Cottonseed oil was added to the ABZ nanoparticles, and the mixture was sonicated for 30 seconds with 40% of power. The dispersion was vortexed and the sonication was repeated for another 1.5 minute. 1-stearyl alcohol was added to this dispersion while heating plate at 70° C. and 1000 rpm. The vial was removed from the stir plate after the stearyl alcohol had melted and placed at room temperature to allow gel formation.

### Pharmacokinetic Study of Oleopastes in Rats

The in vivo pharmacokinetic study was carried out on 2 groups of Sprague Dawley rats. Specifically, the testing group consisted 3 rats assigned with the oleopaste using ABZ nanoparticles without solubilizer above, while 2 rats were used in reference group to test a commercialized tablet. Rats were fasted overnight before the treatments. In the testing groups, the oleopaste was suspended in water then administered by syringes into rat mouths at the dose of 5.7 mg/kg. Similarly, ABZ tablet was crushed into powder and dispersed into water before being administered orally at the same dose. Blood was sampled after 0.25, 0.5, 1, 2, 3, 4, 6 and 24 hours, respectively. At each time point, a maximal blood volume of 500 uL was collected at the lateral tail vein by using a butterfly needle tip and a syringe. Then the whole blood volume was transferred from the syringe into a red-topped serum seperator tube immersed in an ice bath. In order to clot the blood, the tube content was shaken a few times in the tube and leave it undisturbed up to 30 mins at room temperature. The serum was collected as the supernatant after the clotted blood was spun down at 2,000×g, 4° C. for 10 minutes. Finally, serum samples were stored at −80° C. before being analysed later.

The serum was thawed and prepared by protein precipitation before Ultra-Performance Liquid Chromatography-Tandem Mass Spectrometry (UPLC-MS/MS) analysis. Stock solution of ABZ and its metabolites, i.e. ABZSO₂ and ABZSO, were prepared in methanol at a concentration of 500 μm/ml. A twelve-point internal calibration curves of each testing substance ranging from 1.25-5000 ng/ml were prepared. Besides, 80 μL of 250 ng/mL carbamazepine in acetonitrile, i.e. internal standard, was constantly added into calibration standards, blanks and testing samples. After internal standard was added, testing samples were centrifuged at 13000 rpm for 10 minutes. 100 μL of supernatant was pipetted into a 96-well plate containing 100 μL of water.

The UPLC-MS/MS analysis was undergone on a Waters ACQUITY UPLC®-I-Class System aligned with a Waters Xevo® TQ-S mass spectrometer (Waters Corporation, Milford Mass.). Liquid chromatographic separation was performed on an Acquity UPLC® BEH C18 (50×2.1 mm, 1.7 μm particle size) column at 50° C. 2.0 μl of each sample was injected into the system then ionized by electrospray ionization (ESI) in the positive ionization mode. The mobile phase composition included a mobile phase A using aqueous 0.1% formic acid and 10 mM ammonium formate solution, and a mobile phase B consisting of acetonitrile: the solution of 10 mM ammonium formate and 0.1% formic acid solution (95:5 v/v). The gradient elution mode was programmed as such: The initial composition, 80% mobile phase A, was held for 0.50 minutes. The mobile phase composition was then changed linearly to 0% of mobile phase A and 100% of mobile phase B until 2.50 minutes. The composition was held constant at 100% of mobile phase B until 3.50 minutes. At 3.51 minutes the composition returned to 80% of mobile phase A, where it remained for column equilibration for the duration of the run, ending at 5.00 minutes. The flow rate was constantly kept at 0.6 ml/min during the whole elution time. Waters MassLynx 4.1 software was used for data acquisition and analysis. The mass to charge transition (m/z) used to quantitate ABZ was 266.184>234.129 and 237.191>194.206 for internal standard carbamazepine. The mass to charge transition (m/z) used to quantitate the metabolite ABZSO was 282.179>240.110 and 298.174>266.186 for metabolite ABZSO₂.

### Measurement of ABZ Solubility in Various Solubilizers

The solubilizer where 50 mg of ABZ could be most solubilized was screened out of the panel of 9 different candidates (Peceol, Capryol 90, Maisine CC, Plurol Oleique CC 497, Labrasol®ALF, Labrafac™ lipophile WL 1349, Labrafil® M 1944 CS, Labrafil® M 2125 CS and Lauroglycol FCC. After dispensing 50 mg of ABZ into each eppendorf vial, 1 g of each candidate was added and vortexed so that drug was evenly dispersed. Then place those vials onto a tray covered from light on a horizontal shaker for 24 hours. After 24 hours of continuous shaking, vials were centrifuged at 4000 rpm in 10 minutes. Vials with 2 separated phases among which one is clear were selected so that the clear supernatant was taken out and diluted in ratio of 1:10 with methanol.

Likewise, the solubilizer where 100 mg of ABZ could be most solubilized in simulated intestinal fluid (SIF) was screened out of the same panel of 9 different candidates (Peceol, Capryol 90, Maisine CC, Plurol Oleique CC 497, Labrasol®ALF, Labrafac™ lipophile WL 1349, Labrafil® M 1944 CS, Labrafil® M 2125 CS and Lauroglycol FCC. 100 mg of ABZ, 1 g of each solubilizer and 39 ml of SIF were added into each Falcon tube. Then these tubes were vortexed so that drug was dispersed evenly. Again, they were put on horizontal shaker for 24 hours. After 24 hours, they were centrifuged at 4000 rpm in 10 minutes. Again, tubes which have clear middle phase were chosen. Finally, this clear aliquot was diluted with methanol at ratio of 1:10.

Synthesis of ABZ Oleopaste with Selected Solubilizers

Oleopaste containing ABZ powder is prepared according to the below table:

TABLE F

| Ingredients | % w/w | Amount (mg) | |
|---|---|---|---|
| ABZ | 4.0 | 60.0 | mg |
| 1-stearyl alcohol | 6.0 | 90.0 | mg |
| Labrasol ®ALF or Labrafac ™ | 10.0 | 150.0 | mg |
| lipophile WL 1349 | | | |
| Cottonseed oil | q.s. | 1200.0 | mg |

First, 1-stearyl alcohol, Labrasol®ALF or Labrafac™ lipophile WL 1349 and cottonseed oil are weighed into glass vial, accordingly. Then this glass vial containing a stir bar was put on a heating plate at 70° C. and 1000 rpm till being melted. Next, ABZ powder was added into this hot melt liquid and taken off this heating plate after being evenly dispersed in hot oily vehicle.

Oleopaste containing ABZ nanoparticles (formulation 2 from Table 1.1) was prepared according to the below table:

TABLE G

| Ingredients | % w/w | Amount (mg) | |
|---|---|---|---|
| ABZ nanoparticles | 8.0 | 120.0 | mg |
| 1-stearyl alcohol | 6.0 | 90.0 | mg |
| Labrasol ®ALF or Labrafac ™ | 10.0 | 150.0 | mg |
| lipophile WL 1349 | | | |
| Cottonseed oil | q.s. | 1140.0 | mg |

ABZ nanoparticles were weighed into vials. Surfactants and cottonseed oil were added to the vial, and the mixture was probe sonicated as described before. Following complete dispersion, the mixture was heated to 70° C. on a stir plate. 1-Stearyl alcohol was added to the hot mixture. On melting of the stearyl alcohol, the mixture was removed from the stir plate and allowed to cool to room temperature.

Measurement of Drug Release from ABZ Oleopaste by HPLC:

250 mg of oleopaste which is equal to 10 mg of ABZ was weighed into Falcon tubes. Then 18 ml of lipolysis medium was pipetted and the whole formulation immersed in medium was dispersed in 10 minutes. After that, 2 ml of pancreatin solution was complemented. 90-minute time course started immediately when this pancreatin solution was added. The whole release studies were carried out in a shaker chamber at 37° C. and 200 rpm. At certain time points of 15, 30, 45 and 90 minutes, 1-ml aliquots were taken out from these Falcon tubes into microcentrifuge tubes containing 5 μl of 1M 4-bromophenylboronic acid. Then, those small tubes were loaded into centrifuge at 21000 g in 30 minutes. If possible, keep tubes or vials at 37° C. while preparing samples. After centrifugation, clear middle liquid phase was collected and filtered through 0.45 p.m pore sized syringe filters into a new set of microcentrifuge tubes. These aliquots were then diluted with methanol at ratio of 1:1 and place them at −4° C. overnight as whitish precipitation could be detected. Thus, tubes were centrifuged again at 10000 rpm in 10 minutes. The clear supernatant was collected into HPLC vials.

### Results

### Stability of AZT During Manufacturing of Oleogel

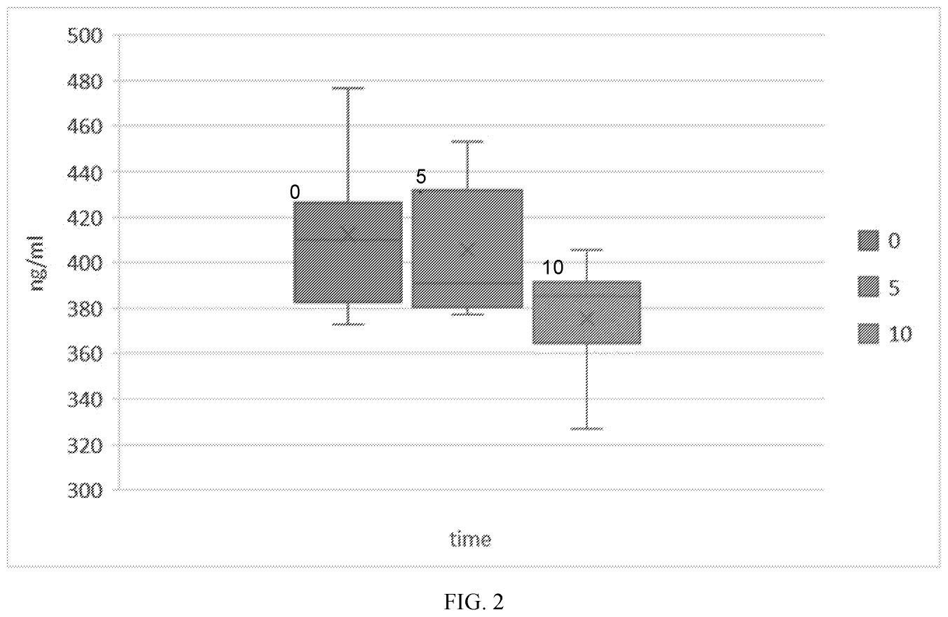

Seven-point internal standard calibrations are established to determine AZT concentrations in each testing sample. In each condition there are 6 replicates of AZT concentrations under testing. The concentrations of AZT recovered following 5 and 10 min of heating at 90C are shown in FIG. 2. A one-way ANOVA statistical test was performed to compare the effect of heating temperature at 90° C. on AZT concentrations after 0, 5 and 10 mins. A conclusion was drawn that there was no statistical effect of temperature on AZT concentrations at the α=0.05 for the three conditions [F(2,15)=1.977, p=0.173]. In other words, it was proved that AZT can be safely heated in oil at 90° C. in either 5 or 10 mins without any statistical reduction of AZT. However, as the oily phase containing AZT with a solid gelling agent is emulsified smoothly within 5 mins, it is meaningful to prolong this step within 5 mins at 90° C.

Drug Release from AZT Oleogel

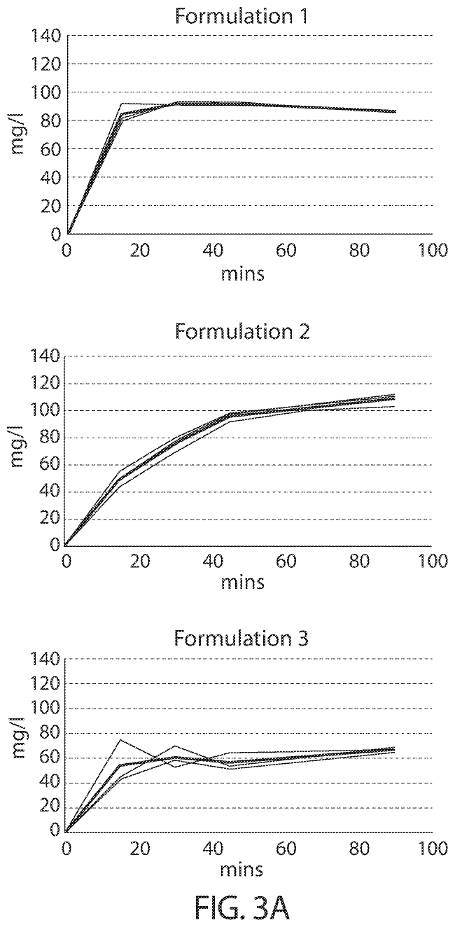

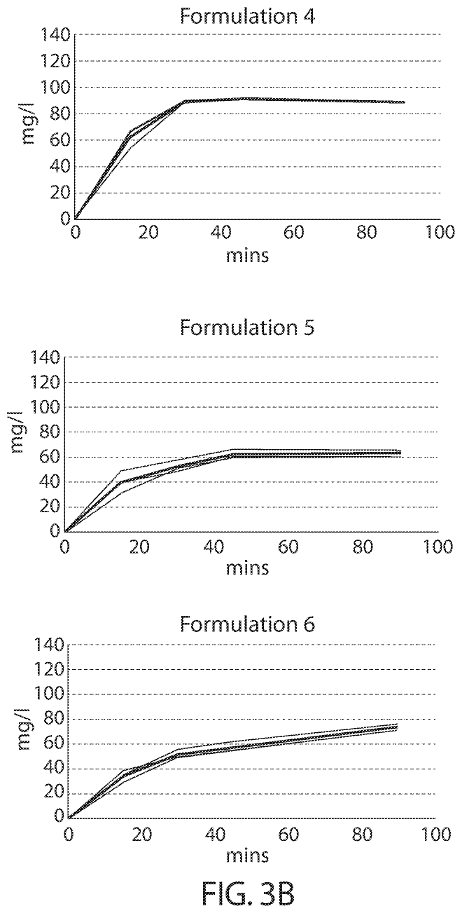

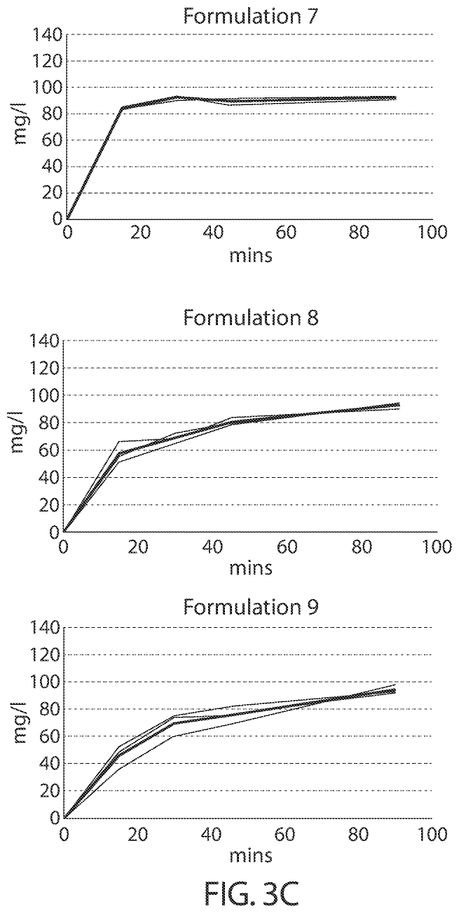

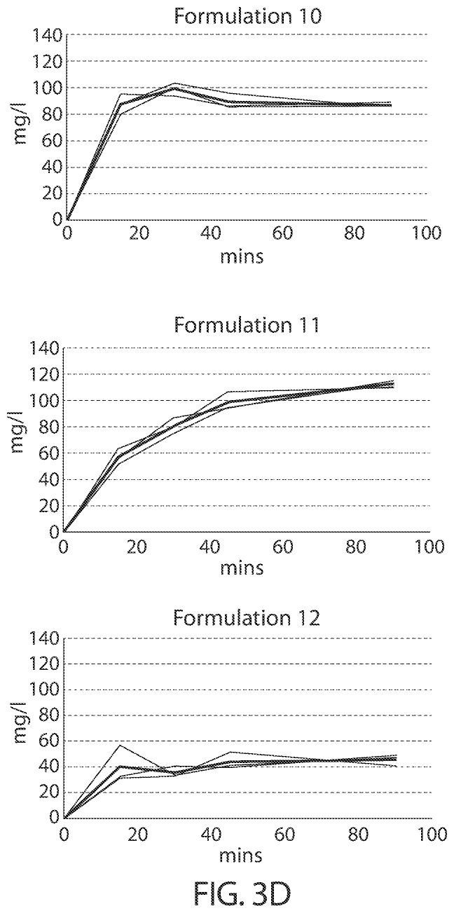

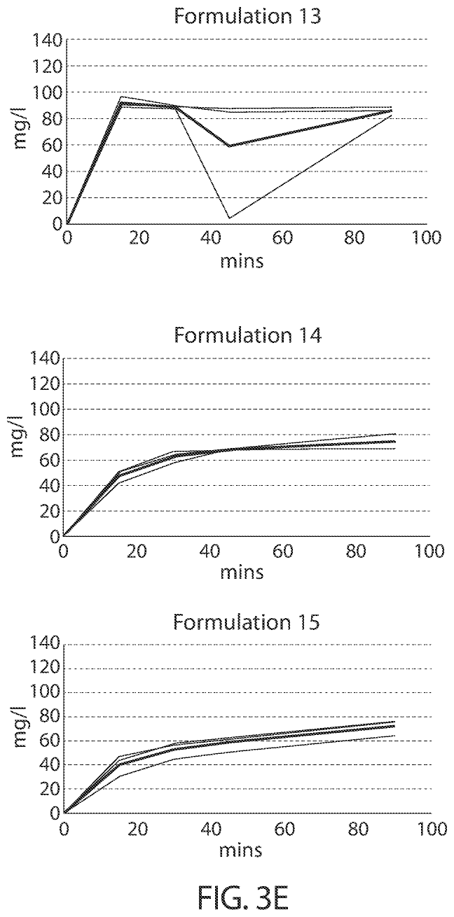

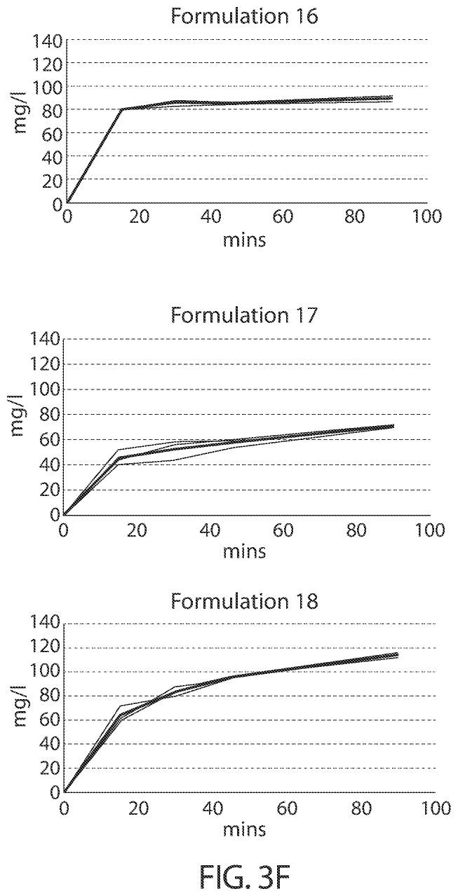

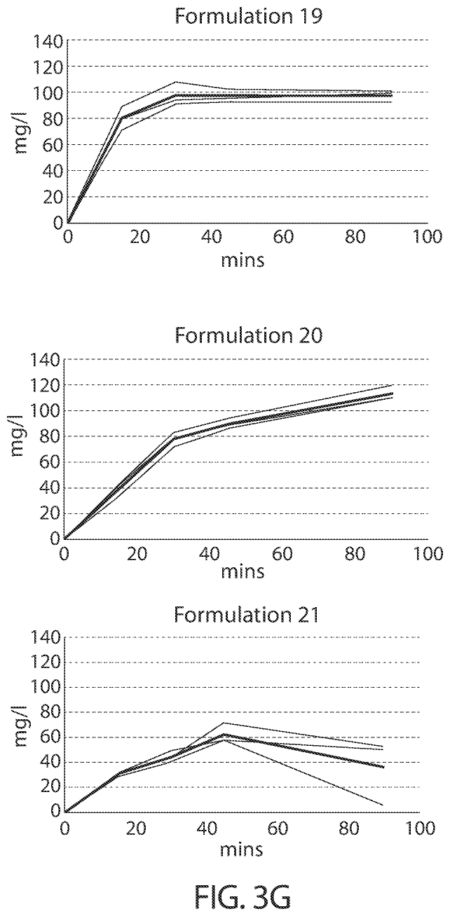

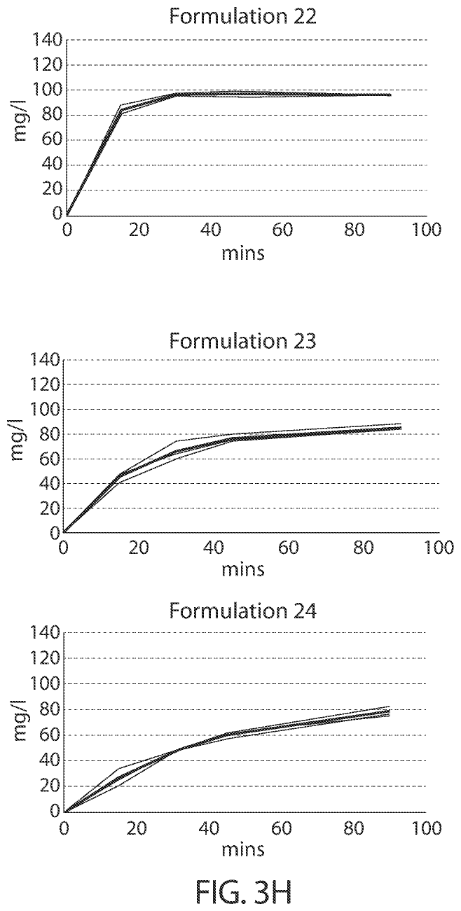

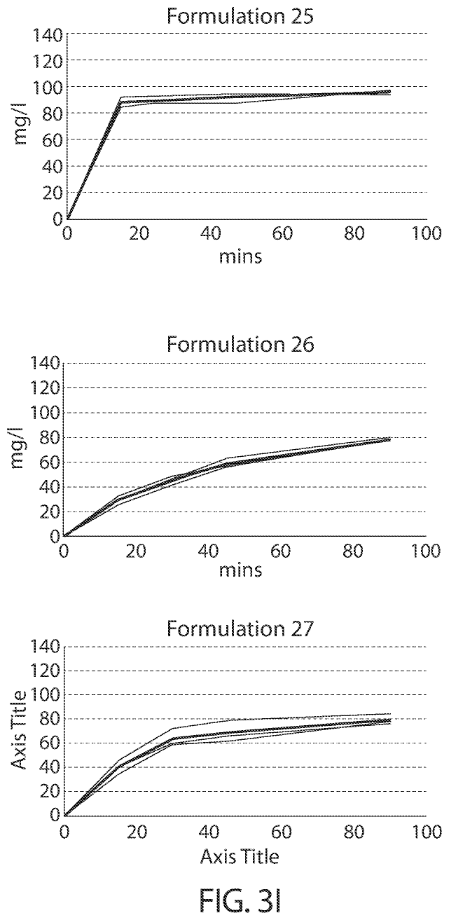

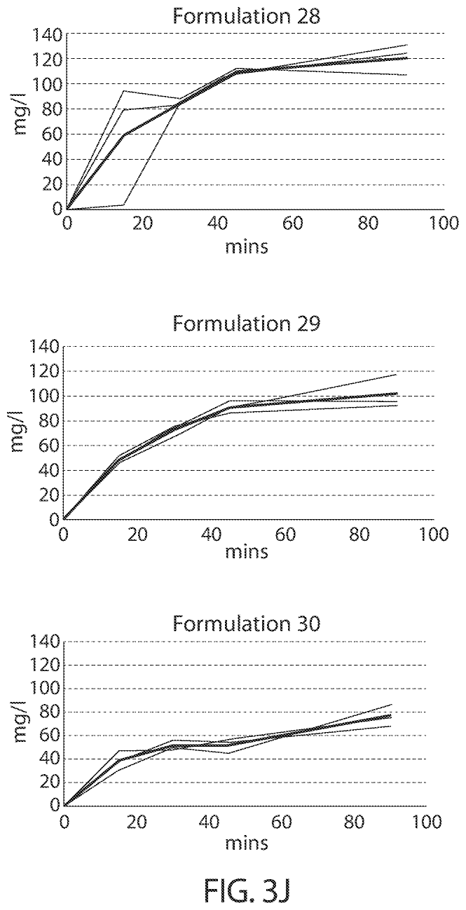

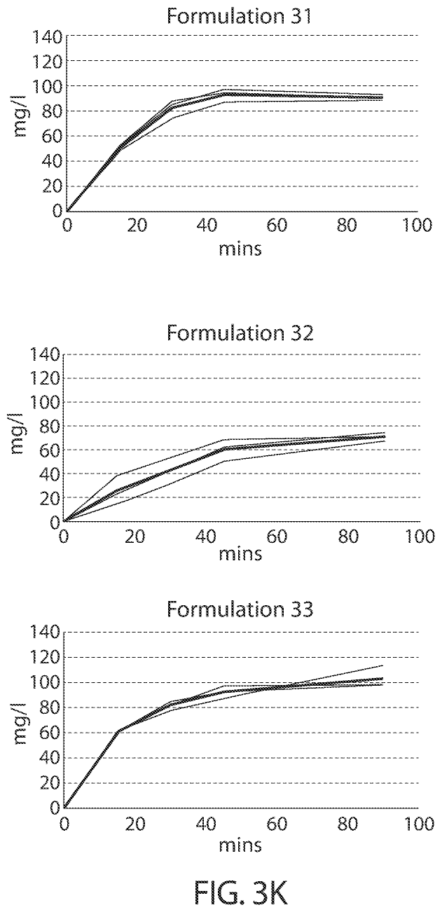

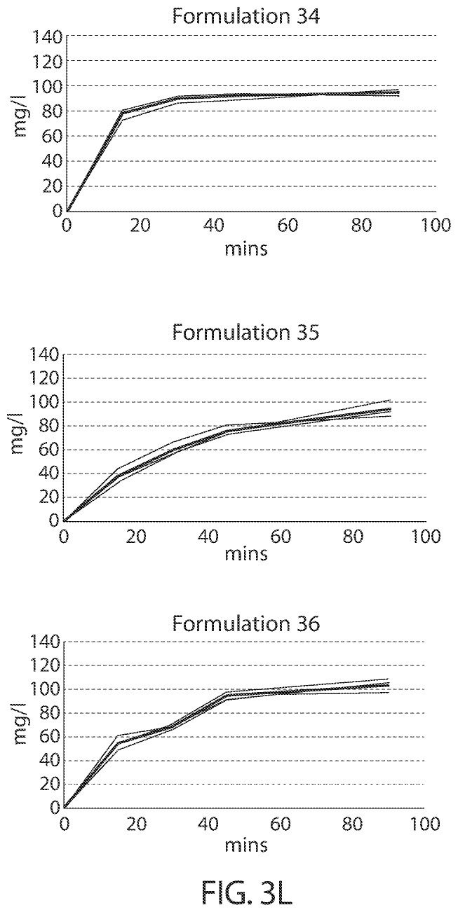

AZT aliquots from release studies of 36 formulations are withdrawn at 4 specific time points then quantitively measured by LC-MS. As a result, 36 average curves of release concentrations over 90 minutes are obtained in FIGS. 3A-3L.

In general, some of oleogels, namely formulation 4, 5, 7, 12, 16, 19, 22, 25 and 34, reached plateau phase within 90 minutes while the others did not reach saturation in the duration of the experiment. Specifically, among them, concentration curves over time of formulation 2, 3, 6, 8, 9, 11, 14, 15, 16, 17, 18, 20, 23, 24, 26, 27, 28, 29, 30, 32, 33, 35 and 36 remained in the upward trend, and their plateaus are estimated to lie beyond 90 minutes. Besides, there are some oleogels, i.e. formulation 1, 10, 13 and 21, that showed a fall in concentrations following an initial peak.

Rate of drug liberated from formulations in early minutes of release course, i.e. the first 15 minutes, can make a decisive impact on the liberation rate of formulation. Indeed, this rate of concentration change over time or the slope of concentration curve in this specific time range can help categorize a formulation into immediate versus sustained liberation type. The steeper the slope in the first 15 minutes compared to other later time intervals is, the higher chance a formulation fits into the immediate release group. In addition to that, the level of plateau concentrations or the near-equilibrium concentrations in later release stage, and the duration from onset to these equilibria also play roles as well. Hence, formulation 7, 16, 19, 22, 25, and 34 appear to release immediately after 15 minutes. Meanwhile, formulation 3, 5, 6, 12, 15, 17, 24, 26, 27, 30 and 32 may be sustained-release oleogels although some formulations have not yet approached a plateau yet.

According to AZT concentrations curves from 36 different formulations, areas under the curve (AUCs) from each release profile are calculated using the trapezoidal rule as in FIGS. 3A-3L. By definition, the AUC expresses drug concentration as a function of time (mg·h/l) as well as a measure of drug released from formulations regarding time. More interestingly, the formulation combined by beeswax, cottonseed oil without any solubilizers gives the highest AUCs (136.63 mg·h/l). The formulation made of cottonseed oil, candelilla wax and Maisine CC showed lowest AUCs with the average of 58.17 mg·h/l

### Effect of Oil on Drug Release

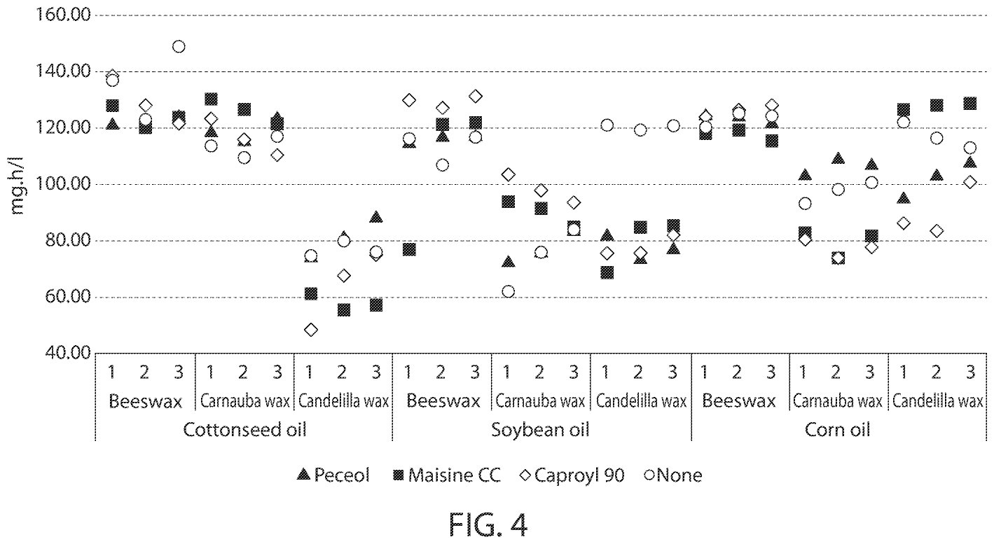

The effect the choice of oil had on drug release (FIG. 4) was studied. Cottonseed oil delivered the best results. Most formulations containing cottonseed oil resulted in high AUCs. The one exception was when cottonseed oil formulations were mixed with candelilla wax. For soybean oil and corn oil, it was more difficult to generalize their effects on AZT release. For formulations containing soybean oil and corn oil, AUC was highest when beeswax was used as the gelling agent. However, use of other gelling agents led to a decrease in AUC.

### Effect of Solubilizer on Drug Release

Assessment on effects of solubilizers, i.e. Peceol, Maisine CC, Caproyl 90 and none, was carried out throughout the screening panel of FIG. 4. Formulations without any solubilizers proved to be the best selection. Six out of 9 formulations without solubilizer showed high AUCs. This interesting phenomenon of not using solubilizers is reinforced when zooming in into a specific group of 4 formulations containing soybean oil and candelilla wax with 4 types of different solubilizers. Here, the formulation lacking solubilizer releases the highest amount of AZT.

Among the 3 solubilizers, regardless of oils and gelling agents, formulations using Maisine CC appear to be the runner up behind no solubilizer.

### Effect of Gelling Agent on Drug Release

A closer look on the impact of 3 different gelling agents, i.e. beeswax, carnauba wax and candelilla wax, on AUCs of 36 formulations in the screening panel of FIG. 4 are taken. Out of them, regardless of solubilizers and oils, oleogels with beeswax consistently produced formulations with higher AUCs. Meanwhile, AUCs of carnauba wax and candelilla wax are not easily forecasted to give high or low values after 90-min release studies.

### In Silico Pharmacokinetic Modelling of AZT Oleogels for the Whole Screening Panel

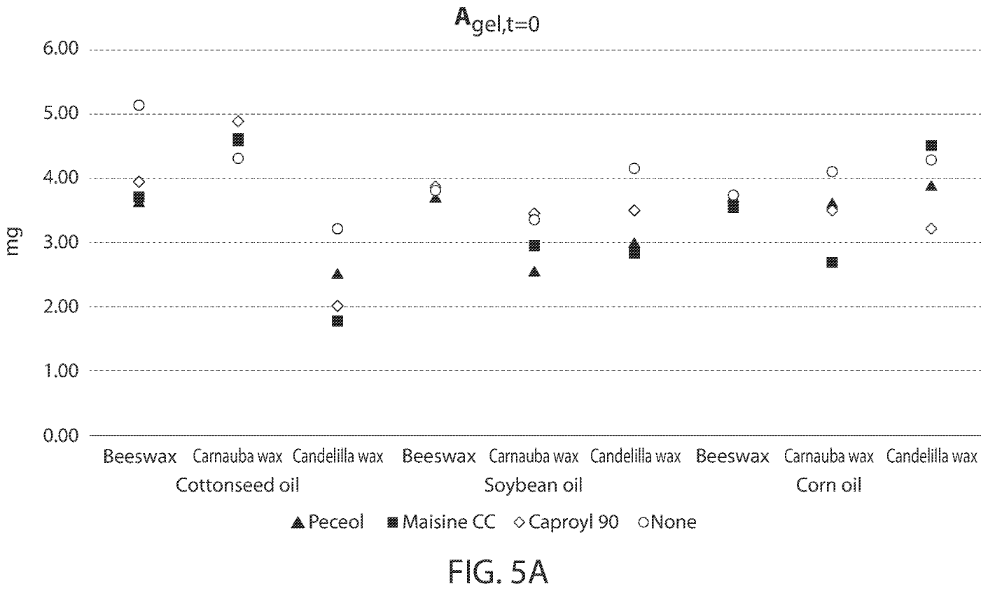

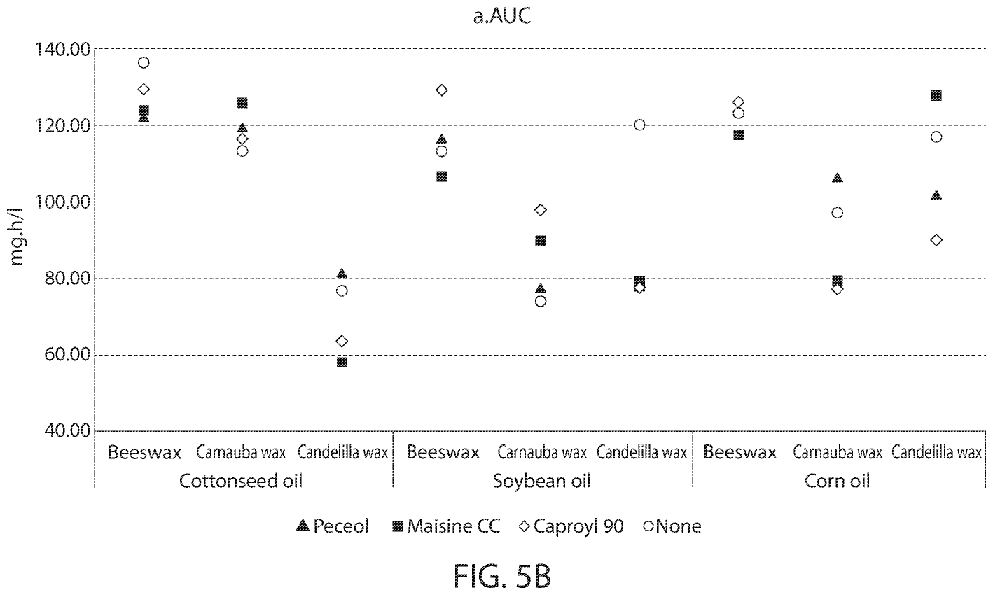

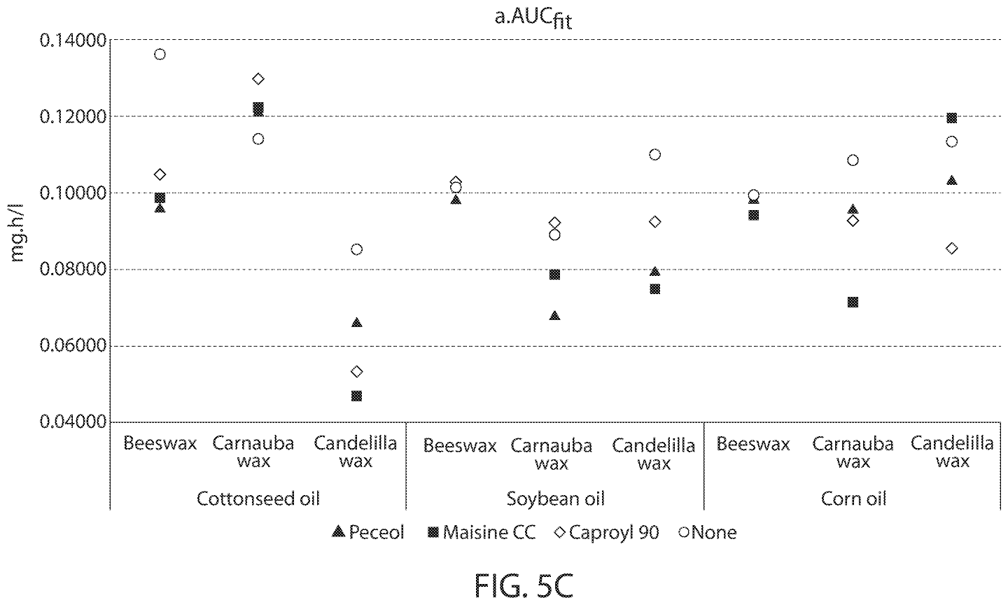

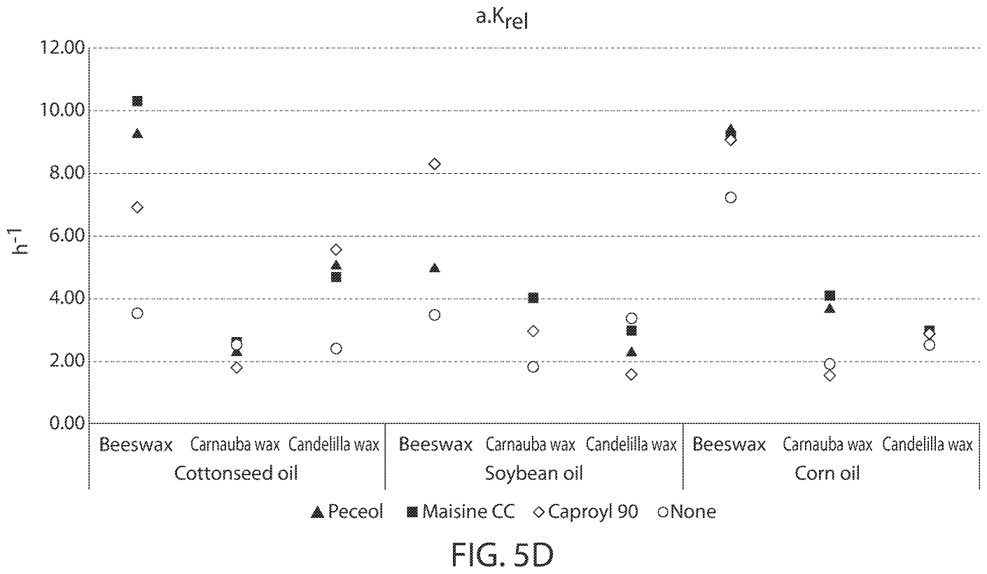

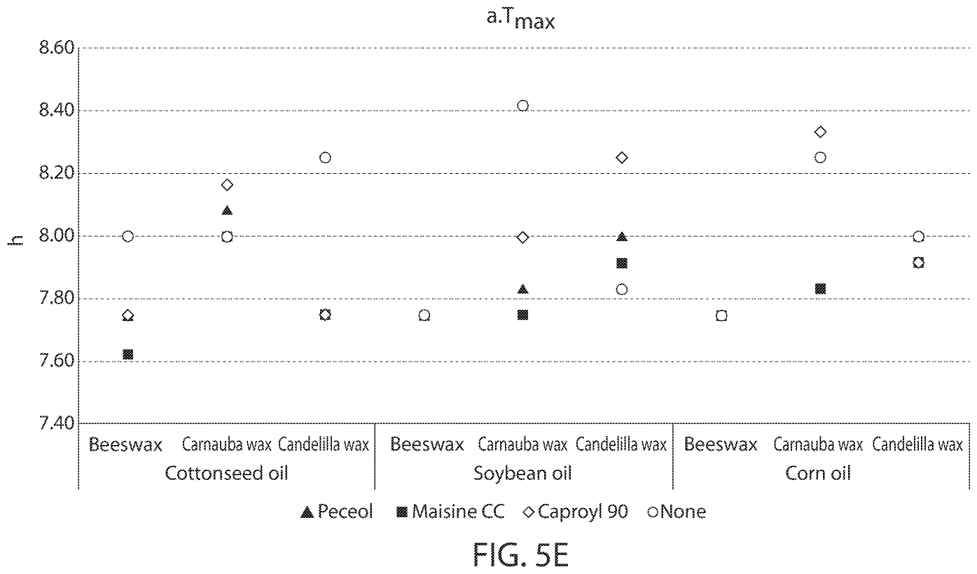

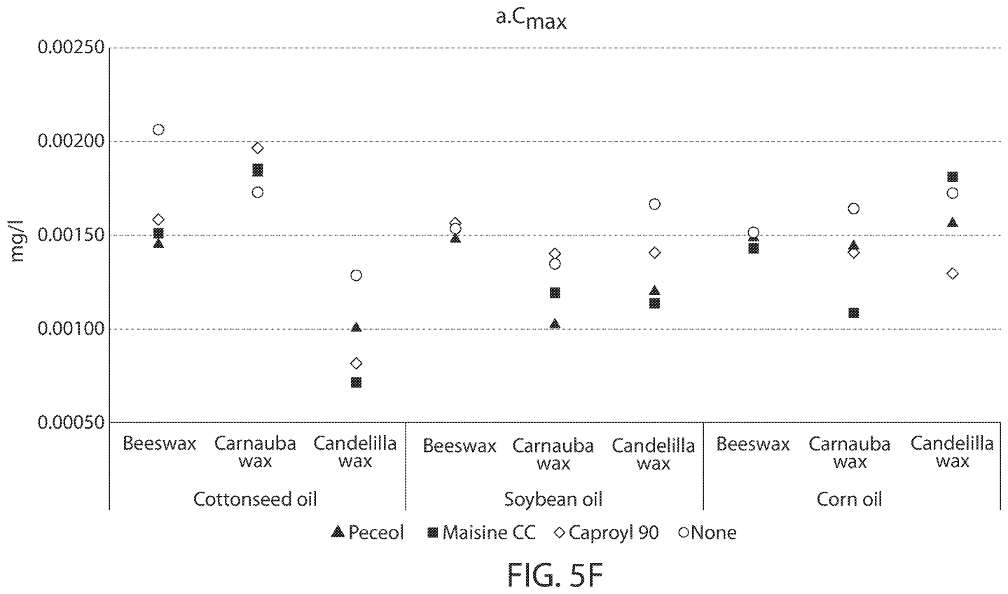

Definite integral of A_{gel}, A_{git} and A are calculated by R program in respect to tin the range from 0 to infinity. However, the amount of drug after 5 times of T_{1/2} was approximated as also approaching the trivial drug amount left in the system at t=∞. Thus, the upper and lower limit of tin these integral coincide in the range from 0 to 250. Some pharmacokinetic parameters, i.e. average released AUC, average A_{gel,t=0,} average AUC_{fit}, average Kᵣₑₗ, average Cₘₐₓ and average Tₘₐₓ are shown in FIGS. 5A-5F.

The highest rank in some pharmacokinetic parameters, i.e. average A_{gel,t=0 ,} average AUC_{fit} and average Cₘₐₓ in pharmacokinetic modelling match with the best formulation screened out from in vitro release studies. Indeed, the best oleogel made of cottonseed oil, beeswax without any solubilizers, i.e. formulation 28, proves to possess highest values of average A_{gel,t=0} (5.12 mg), average AUC_{fit} (0.13643 mg·h/l) and average Cₘₐₓ (0.00207 mg/l). On the other extreme, the lowest values of average A_{gel,t=0} (1.78 mg), average AUC_{fit} (0.04731 mg·h/l) and average Cₘₐₓ (0.00072 mg/l) belong to the oleogel performing worst, i.e. formulation 12, in terms of the released AUC approximated. In addition to analysis of solubilizer on the drug release mentioned, most formulations not using solubilizers and symbolized as red dots also appear to rank as 1^{st} or 2^{nd} positions in terms of average A_{gel,t=0}, average AUC_{fit} and average Cₘₐₓ. In other words, this indicates a trend of not using any solubilizers in oleogels may to lead to a good performer in general. Interestingly, the simulated A_{gel,t=0} varies broadly in the range of 1.78-5.12 mg instead of 4.00 mg of AZT which was actually the amount put into release studies. This explains the actual initial drug amount released into lipolysis media taking into account the partition coefficient of AZT between the oil and aqueous phases.

Apart from those pharmacokinetic parameters mentioned, average kᵣₑₗ and average Tₘₐₓ are not easily estimated from experimental released AUC data because the simulated maxima and minima do not fit into the best and worst formulations mentioned, respectively. Apparently, the oleogel made of cottonseed oil, beeswax and Maisine CC, i.e. formulation 10, offers the highest average kᵣₑₗ (10.32 h⁻¹) and the lowest average Tₘₐₓ (7.63 h), being the formulation that delivers fastest in the screening panel. Values of the average Tₘₐₓ, indeed, does not create a big oscillating range, categorizing formulations into sustained release type ranging from 7.63 h to 8.42 h. Unlike the average Tₘₐₓ, the average kᵣₑₗ creates a value spectrum ranging from 1.54-10.32 h⁻¹, and its trend also does not match with the average Tₘₐₓ's when its maximal and minimal values are not aligned with the corresponding ones in average Tₘₐₓ.

### Comparison Between the Best AZT Formulation and Negative Control

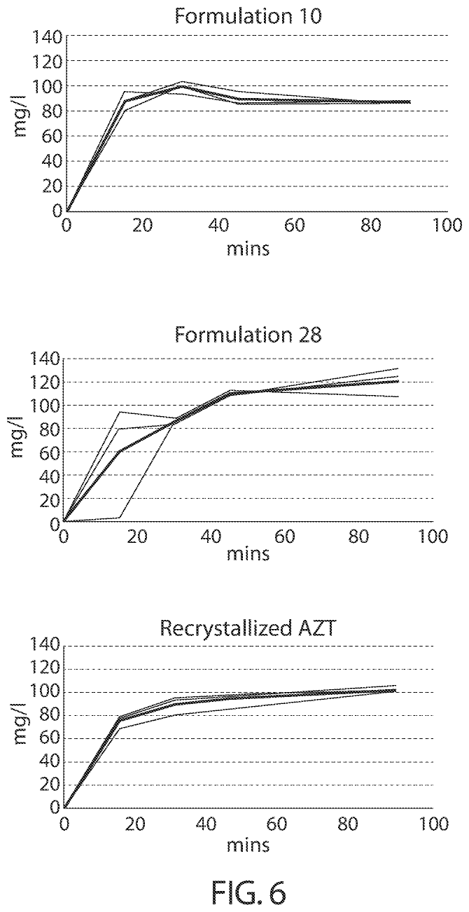

Graphs comparing the 90-minute release course between formulation 10, formulation 28, and recrystallized AZT are shown in FIG. 6. Recrystallized AZT in methanol was chosen as a negative control. In terms of release course, average concentration curve over time of the formulation 28 seems to reach higher values compared to the corresponding ones of the control. At 15-minute time point, values in the formulation 28 is lower and bears a variation among triplicates. For formulation 10, the average concentration curve over time does not look as good as the control.

TABLE 1.3
Simulated pharmacokinetic parameters of the formulation 28 and the control

| | Average | Average | average | average | average | average |
|---|---|---|---|---|---|---|
| | AUC | A_{gel, t=0} | kᵣₑₗ | AUC_{fit} | Cₘₐₓ | Tₘₐₓ |
| | (mg · h/l) | (mg) | (h⁻¹) | (mg · h/l) | (mg/l) | (h) |
| Formulation 10 | 124.13 | 3.71 | 10.32 | 0.09887 | 0.00151 | 7.63 |
| Formulation 28 | 136.63 | 5.12 | 3.52 | 0.13643 | 0.00207 | 8.00 |
| Control | 127.23 | 3.98 | 5.28 | 0.10603 | 0.00161 | 7.75 |

Based on the comparison of the in vitro AUCs and other pharmacokinetic parameters listed in Table 1.3, formulation 28 is the best candidate to proceed next into in vivo study. Also, the formulation 10 could be the runner up due to its outstanding average kᵣₑₗ and other comparable simulated parameters to the control.

### Effect of Polymer and Surfactant on the Size of ABZ Nanoparticles

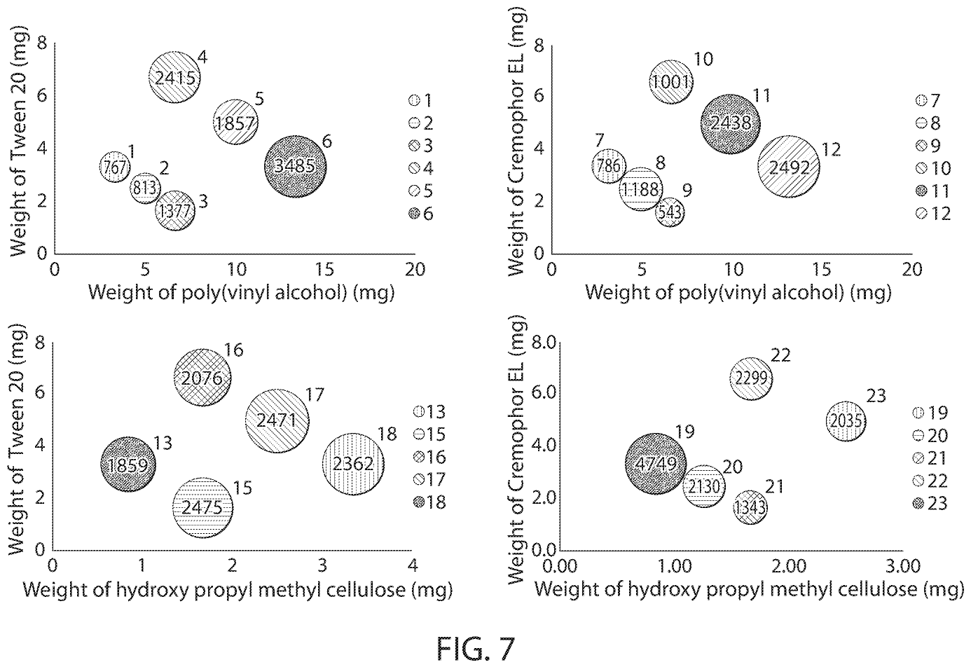

A library of ABZ nanoparticles was prepared by combining drug with polymers [poly(vinyl alcohol) and hydroxy propyl methyl cellulose] and surfactants (Tween 20 and Cremophor EL). Various weight ratios of drug: polymer:surfactant were used. The size of nanoparticles was measured using dynamic light scattering (DLS). A range of nanoparticle size from 543-4749 nm were obtained (FIG. 7). Formulations 1, 2 and 3 (refer to Table 1.2 for details) contained Tween 20 as the surfactant and poly(vinyl alcohol) as the solubilizer showed consistently low particle size, and were taken forward for further characterization.

Drug Release from ABZ Nanoparticles (Non-Oil-Based Formulations)

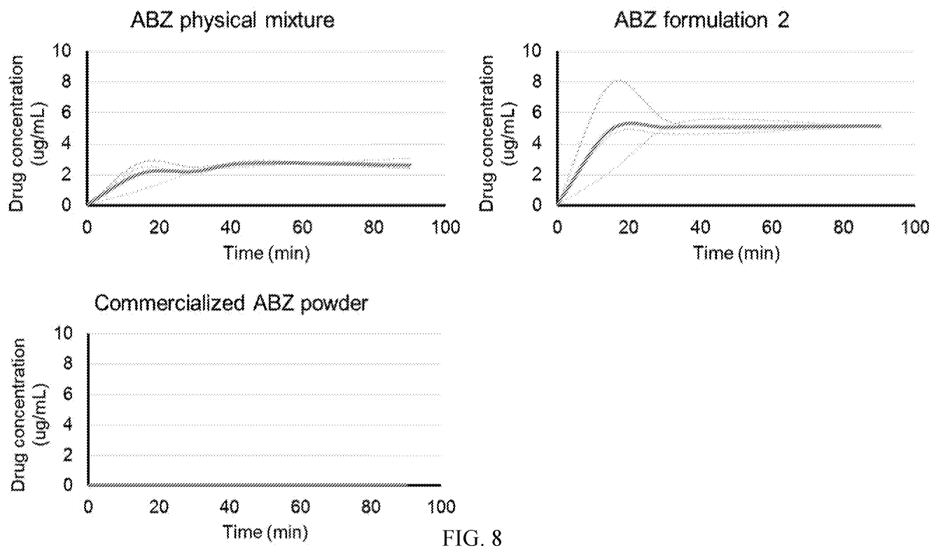

Drug release of albendazole in simulated intestinal fluid was measured. Three formulations were considered: albendazole powder, albendazole nanoparticles prepared using poly(vinyl alcohol) and Tween 20 (formulation #2), and a physical mixture of the drug, polymer and surfactant. ABZ formulation 2 resulted in the best release profile over the 90-minute time course. Although there is a gap of concentrations among 3 replicates within the first 30 minutes, the amounts of ABZ released and solubilized are stabilized and reach plateau at approximately 5.1 μg/ml till the end of release studies (see FIG. 8). This average plateau concentration is approximately double the average value of physical mixture at 2.5 μg/ml. Meanwhile, the ABZ powder is solubilized consistently in lipolysis buffer at 0.05 μg/ml. Formulation of ABZ in nanoparticles can help improve the solubility of ABZ in lipolysis buffer over the ABZ powder and physical mixture.

Drug Release from ABZ Oleopastes
Effect of Nanosizing on Drug Release from Oleopaste without Solubilizers

An oleopaste of ABZ was formulated by incorporating ABZ as powder or as nanoparticles into an oil. Release of ABZ from these formulations in simulated intestinal fluid was measured (see FIG. 9). The oleopaste containing ABZ nanoparticles showed higher drug release in comparison to the ABZ powder containing oleopaste. At the end of the release study, the nanoparticle containing formulations produced a concentration of ˜20 ug/ml. In contrast, the formulation containing ABZ powder resulted in a concentration of ˜10 ug/ml.

### Pharmacokinetics of ABZ Oleopaste

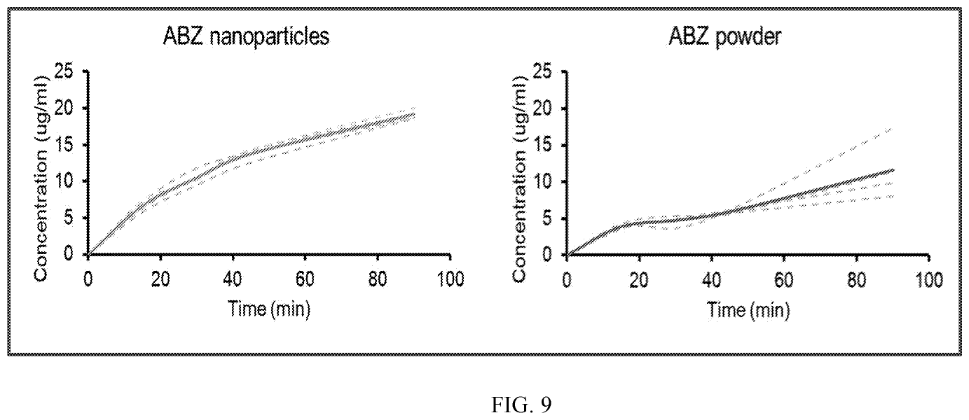

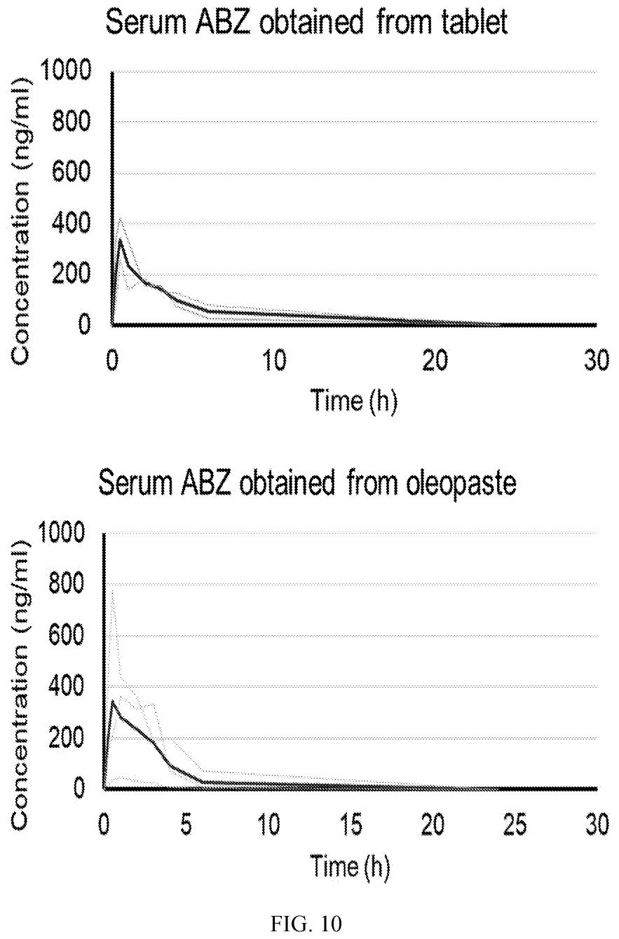

The pharmacokinetics of ABZ administered as commercial tablets or in the oleopaste formulation was assessed. The serum pharmacokinetics of the two formulations is shown in FIG. 10. The pharmacokinetics of the two formulations were comparable. The tablets achieved an average Cₘₐₓ 339.35 ng/ml at 0.5h after dosing. The Cₘₐₓ of the oleopastes was 340.60 ng/ml at the same Tₘₐₓ. Moreover, ABZ is almost completely eliminated at 24-h time point. Among 3 rats receiving ABZ oleopastes, there was variation. This variation could be attributed to the extremely low dosing volumes of oleopastes used in this experiment.

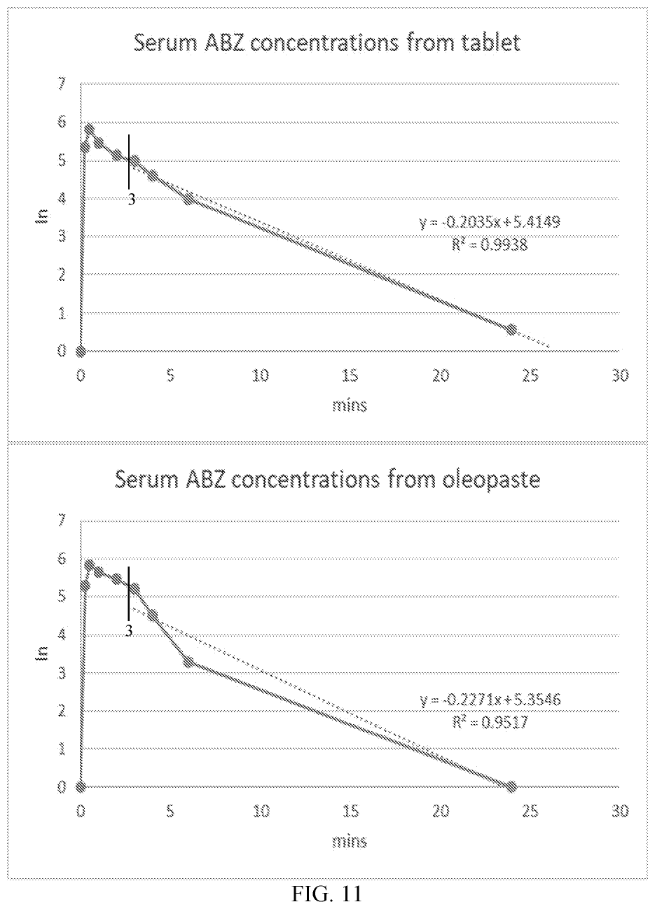

The elimination rate constant and half-life of ABZ for the two formulations was estimated using the terminal pharmacokinetics. According to the concentration curves in FIG. 11, elimination phase of 2 groups is supposed to last between 3 - and 24-hour time points. By using regression line method on those points, elimination constants corresponding to powder and oleopaste are found out as 0.2035 and 0.2271 h⁻¹. Based on these constants and the formula, T_{1/2} of ABZ powder and ABZ oleopaste without solubilizer correspond to 3.41 and 3.05 h.

Using the trapezoidal rule, the serum AUC of ABZ for the treatment groups was estimated. On average, the AUC for the tablets and oleopastes were 1372.00 ng·h/ml and 1214.67 ng·h/ml respectively. As mentioned previously, there was variability in the serum pharmacokinetics of the oleopastes and this was reflected in the AUC analysis.

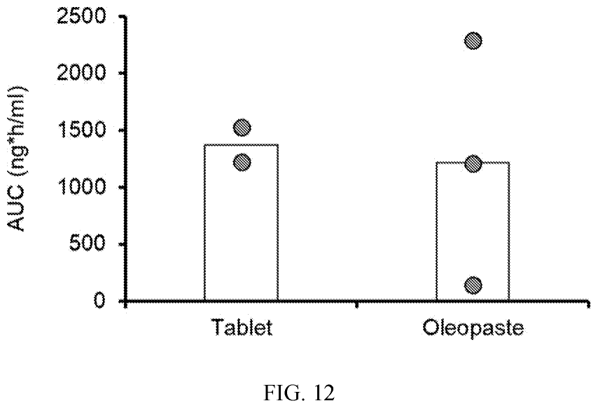

In summary, ABZ released from tablet and oleopaste was comparable (see FIG. 12).

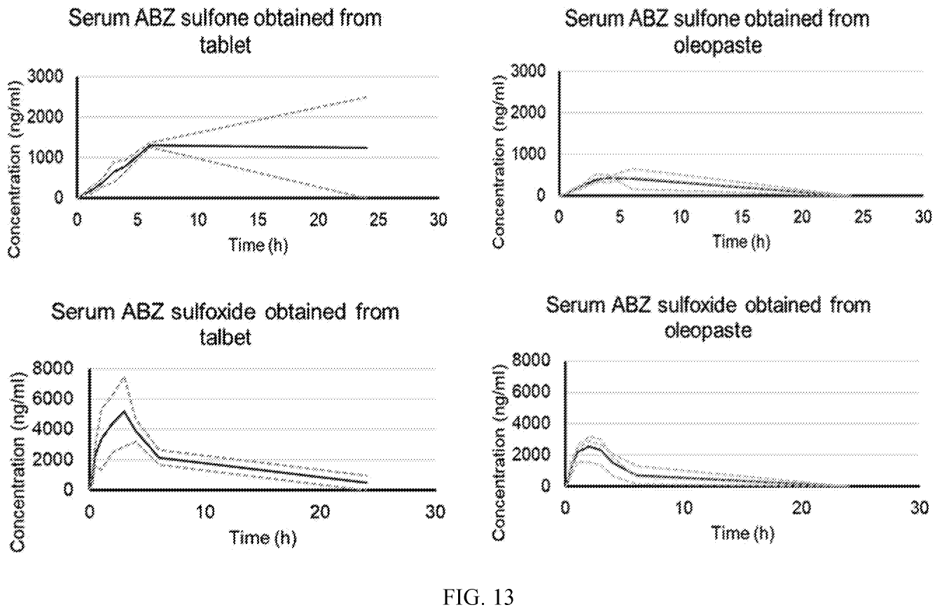

Unlike ABZ with which oleopaste group is recorded with higher variation among individual values, this phenomenon is observed differently in serum ABZ sulfone and ABZ sulfoxide. According to FIG. 13, there is a bigger difference in serum concentrations observed in the tablet group than in oleopaste group. By contrast, serum concentrations in oleopaste group fluctuate in a magnitude not as broad as the tablet group. Apparently, ABZ in tablet group appears to be metabolized more extensively than the oleopaste.

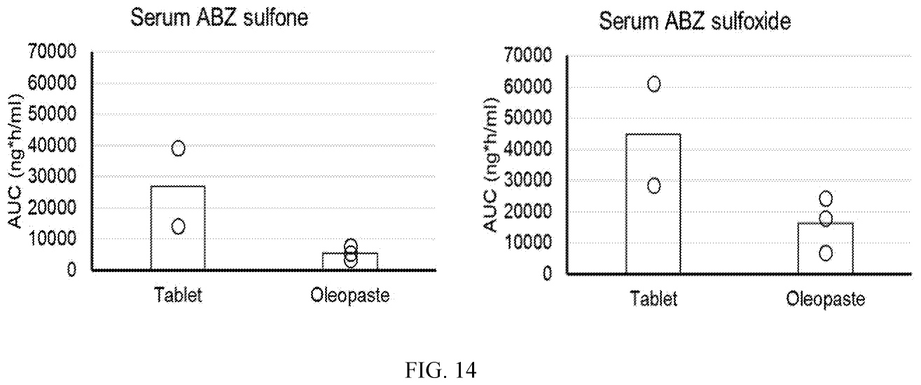

The distinguished value variation in tablet group is also reflected in terms of AUC calculation in FIG. 14. Moreover, the drug metabolite exposure AUCs in tablet group of both ABZ sulfone and ABZ sulfoxide are averagely higher than ones in oleopaste, respectively. Additionally, the serum ABZ sulfoxide, i.e. the primary active ABZ, has higher average AUCs in both tablet (44764.5 ng·h/ml) and oleopaste groups (16364 ng·h/ml) than the serum, inactive ABZ sulfone (26797.2 ng·h/ml in tablet vs. 5545.1 ng·h/ml in oleopaste group). This suggests that ABZ transforms and maintains in the active metabolite at higher proportion than the inactive one in both groups, and the ratio of active:inactive metabolite in oleopaste group is higher in the tablet one in particular.

### Effect of Surfactants on ABZ Solubility in Various Solubilizers

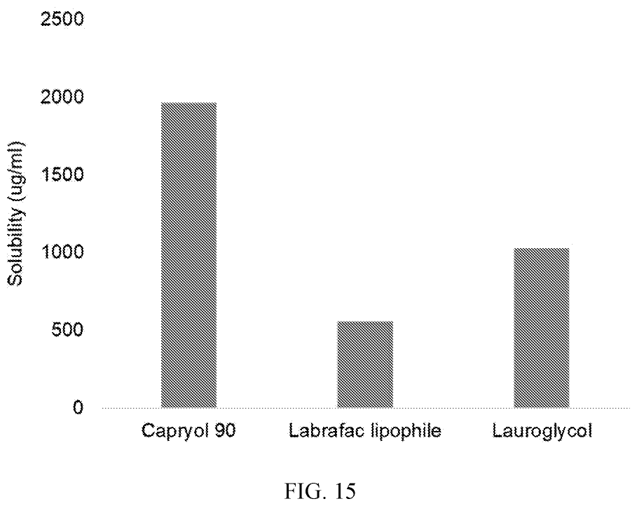

Whether inclusion of lipophilic surfactants would increase the solubility and drug release of ABZ in simulated intestinal fluid was investigated. Average solubility of ABZ powder in solubilizers, i.e. Capryol 90, Labrafac™ lipophile WL 1349 and Lauroglycol FCC is shown in FIG. 15.

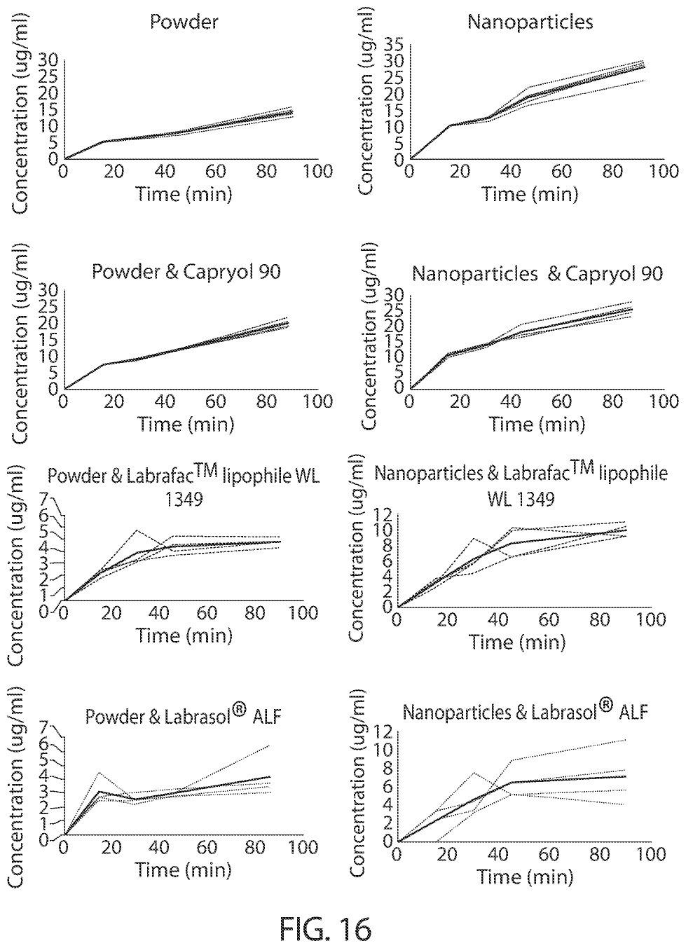

Oleopastes containing either albendazole powder or albendazole nanoparticles mixed with three surfactants—Capryol 90, Labrafac lipophile WL1349 or Labrasol ALF were prepared. Drug release from these formulations in SIF was measured (see FIG. 16). In formulations not containing the surfactants, nanoparticles led to a higher drug release in comparison to ABZ powder. However, when a solubilizer was included the difference between the nanoparticle group and the powder group diminished. This suggests that nanosizing could be avoided if a surfactant is included in the formulation. Unfortunately, there was no further benefit of using the surfactant. In fact, inclusion of the solubilizer led to a slight reduction in overall amount of drug released. This observation is similar to what was observed with AZT.

### Discussion

### AZT Oleogels

The 36 AZT combinations made of varying oils, solubilizers and gelling agents reveal 36 different concentration curves over time in in vitro release studies. Among them, a few formulations are slowly reaching its plateau phase, suggesting that a more accurate picture in drug release could have been obtained by extending the release time more than 90 minutes. Then the equilibrium state between AZT solubilized in lipolysis media and oil-based system as well as maximal releasing concentrations could be determined for all. This may promote the prediction and explanation of pharmacokinetic modelling. Also, technically, because the oleogel could not evenly dispersed in lipolysis media and this capability is varied differently in each oleogel, an unavoidable error in sampling potentially exists, signifying the variation of triplicates of a few formulations.

Collectively, individual effects of oil, solubilizers and gelling agents are concluded. In some instances, AZT oleogels compounded without any solubilizers have higher chance to be more favorable than solubilizer options. It contradicts to established paradigm that solubilizer helps to reduce the contact angle and facilitate the solubilization of drug particles. Normally, solubilizer molecules form micelles at or beyond the critical micelle concentration, creating a vehicle where drug molecules are incorporated inside the sphere or between solubilizer molecules. This controversy may have emerged from the fact that solubilizers compounded in oleogels are also technically oil entities rather than a solubility enhancer in this context. Thus, instead of facilitating the micellization of drug into the lipolysis media, these solubilizer probably increase the lipophilic property of the oil-based system, trapping AZT molecules inside even in the presence of bile salt as a natural surfactant. Therefore, the intricate contribution of solubilizers in a bigger picture involving interactions with other formulation excipients may be reconsidered based on observations regarding AZT oleogels and ABZ oleopastes, as discussed in more detail below.

Regarding effect of oil on AZT drug release, cottonseed oil seems to outstand the other 2 oils, i.e. soybean oil and corn oil. Also, regarding to effect of gelling agents on AZT drug release, beeswax performs better than carnauba wax and candelilla wax. These observed advantages may be explained by the varied component and diverse amounts of each constituent in corresponding oil or wax, which can be evidenced by next experiments.

Furthermore, few formulations indicate that there is a fall in release concentration at a certain time point where the spring-parachute effect comes into play (Bavishi & Borkhataria, 2016). Thanks to this lipid-based system and the solubilization enhancement from lipolysis medium, AZT oleogel increases the drug dissolution, achieving higher energy state of supersaturation. Instead of the phenomenon of sudden precipitation from supersaturation called the spring state, there is a gradual energy relaxation called parachute effect right after the drug concentration lands on the supersaturation state. The AZT release studies show that some oleogels possess this relaxation effect.

In silico pharmacokinetic modelling is deployed to predict the pharmacokinetic profile of AZT oleogels in human. Basically, the best performer concluded from this modelling also aligns with the best observed in in vitro release studies. Besides, taking the kₐ and kₑ from literature, the oleogels seem to liberate drug in a sustained way by which they take longer time to reach serum Cₘₐₓ than a typical oral immediate formulation like Zithromax tablet (Food and Drug Administration, 2011). Average Cₘₐₓ and AUC_{fit} are lower than those in clinical studies of Zithromax tablet (Food and Drug Administration, 2011). This means that certain formulations identified after screening and pharmacokinetic modelling may be superior to the AZT powder form. The initiative may come from the improvement in drug loading of oleogel to increase the serum drug concentrations and other related parameters when a higher supersaturation can be established, affecting to both A_{gel} and kₐ in equations 6,7 and 8. Or to increase the kᵣₑₗ , oleogel could be reformulated with more hydrophilic polymer(s) to create a multipurposeful scaffold, for instance, hydroxypropyl methylcellulose or methyl cellulose combined with xanthan gum (Patel et al., 2014), gelatin and xanthan gum (Patel et al., 2015). Accordingly, owing to the hydrophilic characteristic of the matrix, the oleogel disintegrated more quickly in aqueous media, enhance both the kᵣₑₗ then probably the permeability or kₐ. This hydrophobic oleogel could be developed as an oral sustained release system for other potential drugs as its long simulated Tₘₐₓ and T_{1/2} for AZT was observed.

### ABZ Oleopaste Containing Nanoparticles

An oil-in-water emulsion template using Tween 20 and PVA scaffold in mass ratio 1:1 was synthesized to produce the AZT nanoparticles with approximate nanosize of 813 nm. This new nanoscale entity produced by the ETFD technique proves to perform best in SIF media in comparison to the drug powder and drug physical mixture, emphasizing the combined effect of particle nanosizing and the emulsion excipient over the sole effect of each. Following that, oil-based formulation of ABZ nanoparticles without any solubilizer is prepared and outperformed its counterpart using ABZ powder. An in vivo pharmacokinetic study of the mentioned oleopaste is then carried out on rats using commercialized tablet as its reference, showing different parameters of ABZ and its 2 metabolites. Although the pharmacokinetic profiles of both testing oleopaste and reference are comparable in terms of serum ABZ, parent ABZ molecules are metabolized to ABZSO and ABZSO₂ more extensively in tablet group. As lipid—based vesicles or micelles carrying parent ABZ were predicted to be absorbed into lacteal lymphatic system preferentially, they are likely to bypass the hepatic biotransformation via portal hepatic vein, leading to lower concentrations of the 2 primary metabolites in the oleopaste group compared to the tablet one. This hypothesis will be probably tested by formulating another potential drug which targets lymphatic system or organ to specifically measure drug concentrations from these certain places.

Afterwards, in order to determine the most suitable solubilizer to complete the formulation, a panel of 9 different solubilizers is screened out by the amount of ABZ dissolved directly in each. Of them, ABZ is solubilized most in Capryol 90, and the runner up is Lauroglycol. In parallel, another screening experiment where ABZ is solubilized in each solubilizer and SIF medium, instead. Labrafac™ lipophile WL 1349 is the best, and the next is Labrasol ALF while Capryol 90 and Lauroglycol are undetected. Again, release studies of oleopaste containing ABZ nanoparticles when formulation with Capryol 90 and without solubilizer outperform the other 2 solubilizers. Maybe there are diverse effects of solubilizer on ABZ solubilization when it interacts with different excipients with the underlying mechanism. As discussed above with respect to AZT oleogels, incorporating a solubilizer into oleopaste can adjust the lipophilicity of the system, leading to facilitating or impede the ABZ solubilization from the lipid-based system.

### Section 2: Azithromycin, Lumefantrine and Praziquantel

### Results

### Analysis of the WHO Model List of Essential Medicines for Children

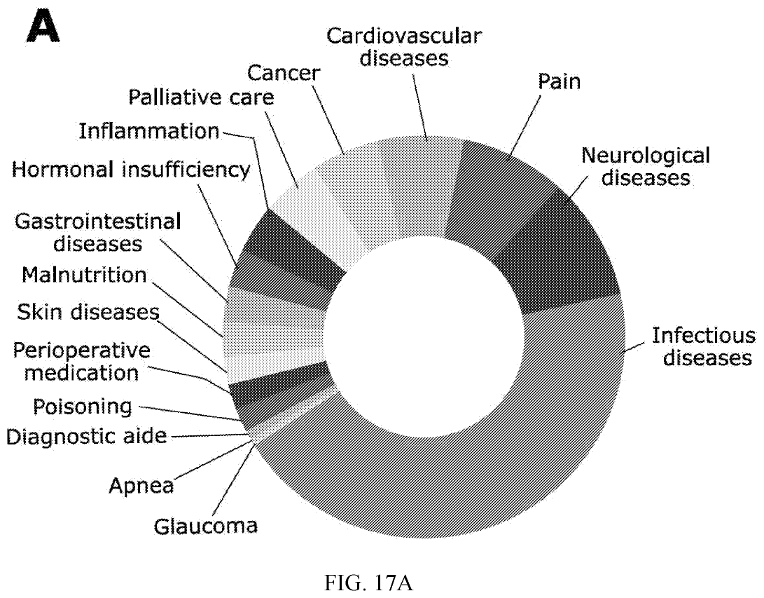

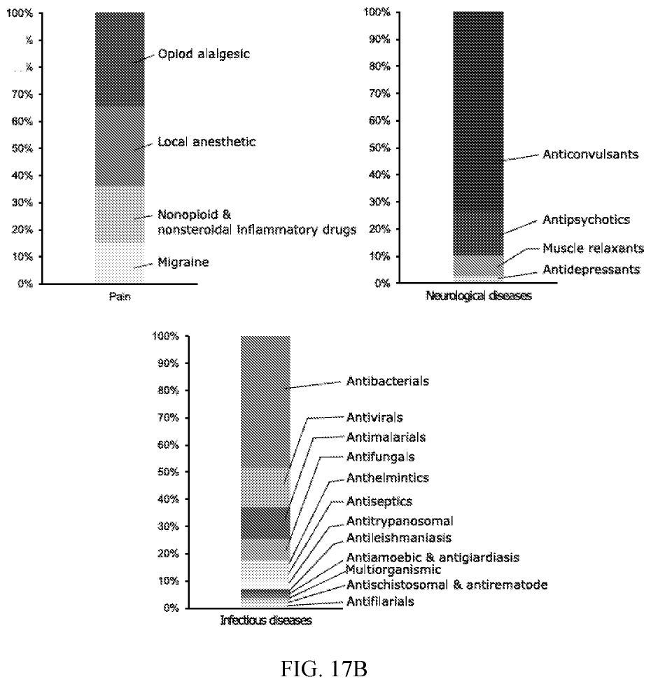

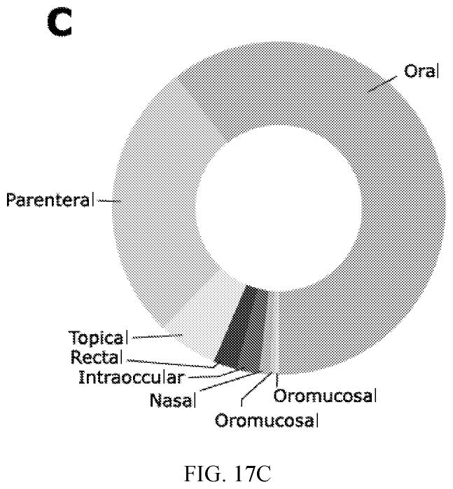

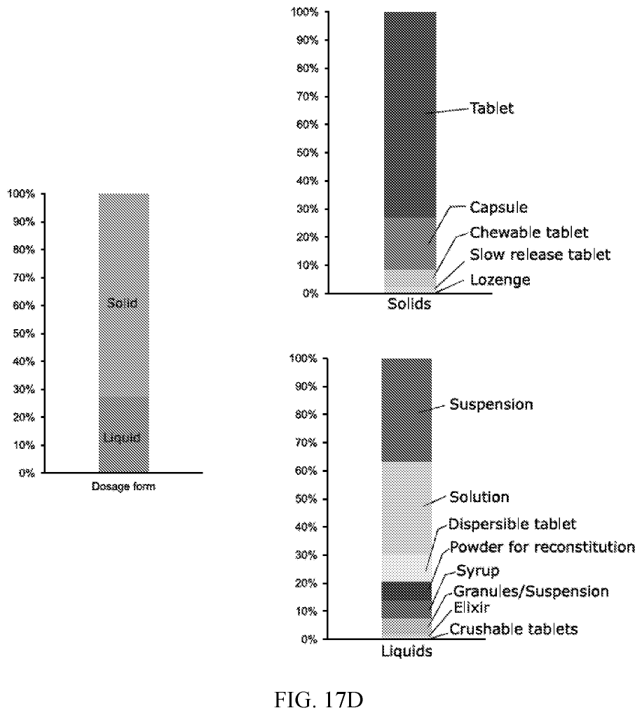

Identifyication of drug categories that would be most vital for care of children were investigated. Hence, the WHO model list of essential medicines for children²⁶, a minimum list of medicines needed by a basic health care system, was consulted. These essential medications were categorized based on their target disease. Drug products intended to treat infectious diseases (44%), neurological diseases (10%) and pain management (8.4%) formed the bulk of the list (FIG. 17A). Medicines for cancer and cardiovascular diseases comprised of >5% of the medication list.

The various drug categories amongst the top three disease areas were then analyzed (FIG. 17B). Amongst infectious diseases, antibacterials were the most commonly listed drug products and comprised of about half of the anti-infectives. Other common anti-infectives included antiviral and antimalarial drugs. Anticonvulsant drug products formed the majority of those listed for the management of neurological diseases. Drug products that could be used for pain management had comparable numbers of opioid analgesics, non-steroidal and non-opioid analgesics and local anaesthetics.

Patient acceptability of drug products is strongly reliant on the route of administration. Hence, the most popular routes of administration for the drug products in this list were studied. Unsurprisingly, about 60% of the drug products were intended to be used orally (FIG. 17C). Other common routes of administration included parenteral (27.2%), topical (5.8%) and rectal (2.3%).

About 72% of the oral products were intended to be consumed as solids (FIG. 17D). This is despite the fact that granules, powder for reconstitution, crushable tablets and dispersible tablets were categorized as liquids due to the change in their physical form right before administration. Roughly, 90% of the solid dosage forms were tablets (immediate and sustained release) and capsules-dosage forms that present a challenge for children to swallow. This suggested that there is a pressing clinical need to design dosage forms that enable drug delivery to children, preferably from a non-invasive route of administration.

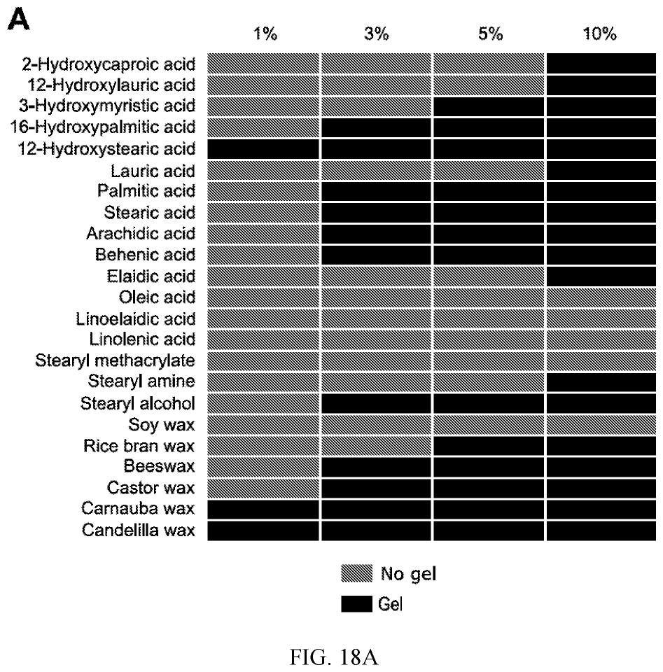

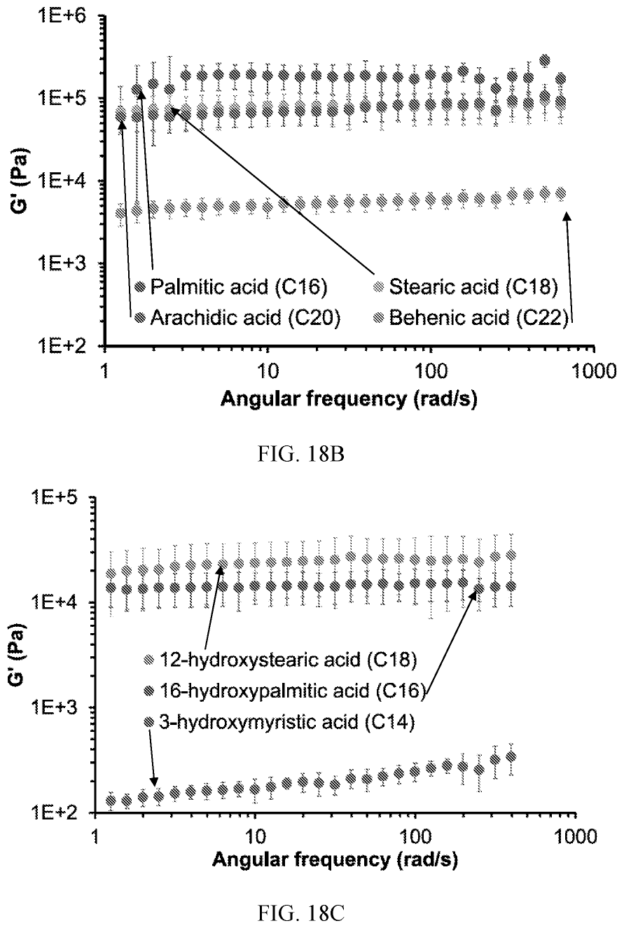

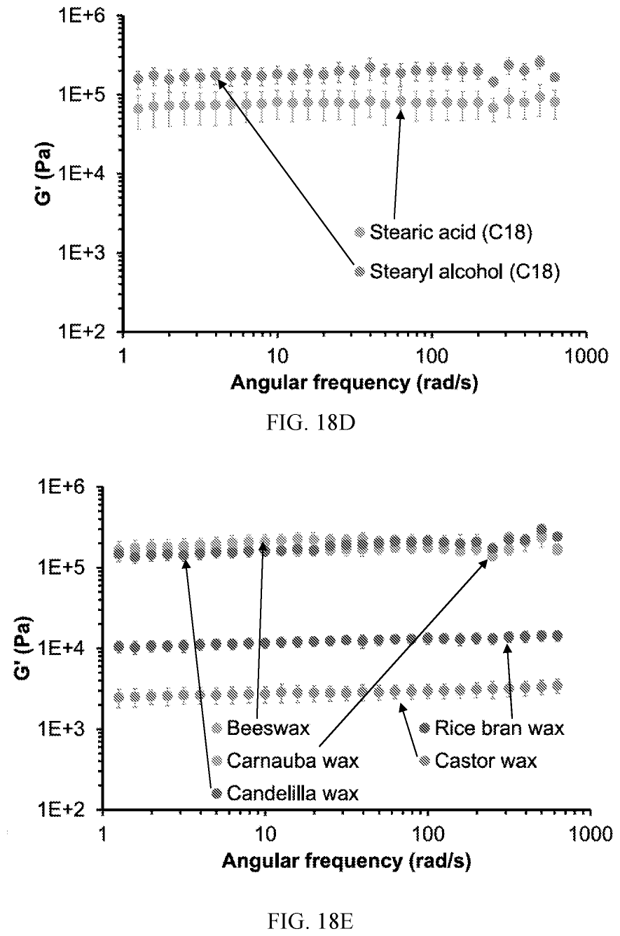

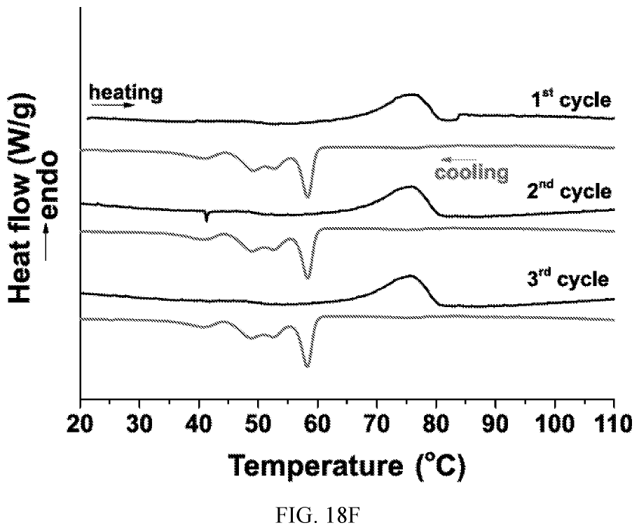

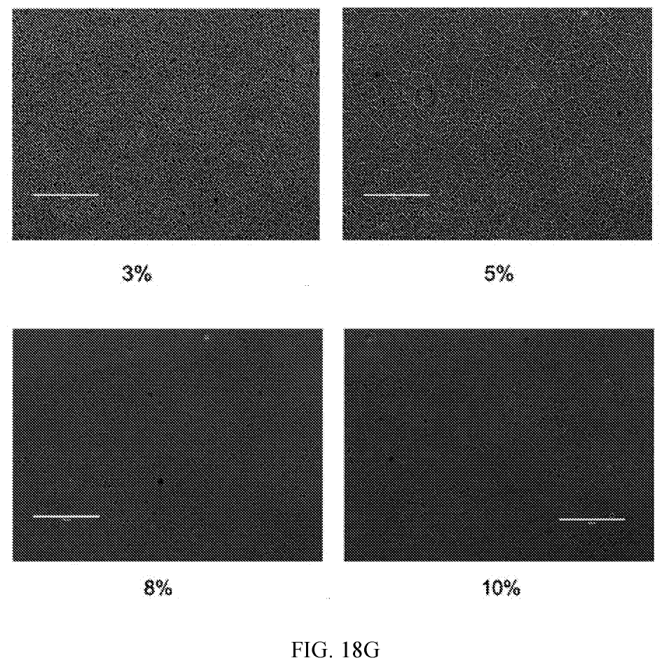

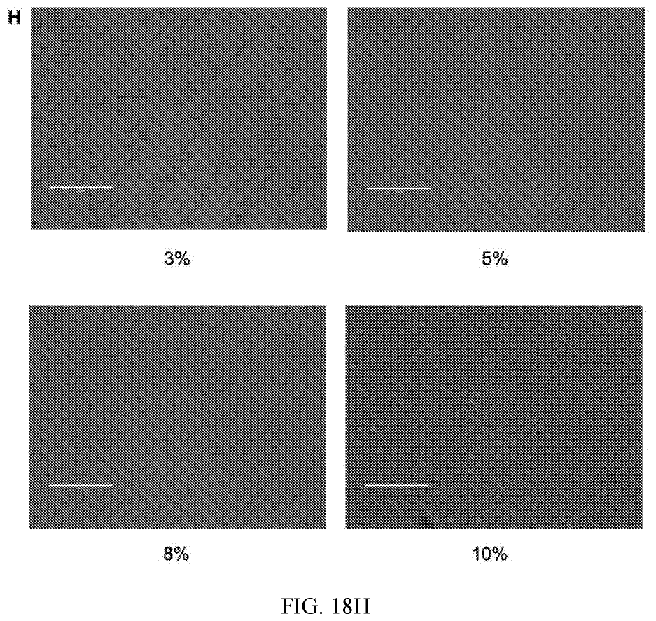

Due to their occurrence in oils and miscibility with oils, gel formation with fatty acids were examined. Formulations were made by heating the oil-fatty acid mixture above the melting point of the fatty acid, followed by cooling to room temperature. Five concentrations of fatty acids were analysed (FIG. 18A). The smallest linear fatty acid tested, lauric acid (C12), formed gels at a concentration of 10%w/w. Long-chain fatty acids such as palmitic (C16), stearic (C18), arachidic (C20), and behenic acid (C22) were capable of forming gels at much lower concentrations (3% w/w). A similar trend was observed with hydroxy fatty acids, in that, chemicals with longer chains formed gels at lower concentrations. Interestingly, when comparing hydroxy stearic acid and stearic acid, it was observed that the hydroxy fatty acid was capable of forming gels at a lower concentration than the fatty acid.

The effect of addition of unsaturated fatty acids on the consistency of oil was also analyzed. Gelling capacity of two isomers of monounsaturated octadecanoic acid viz. elaidic acid and oleic acid were compared. Interestingly, the cis-isomer (oleic acid) was not capable of forming gels in the concentration range tested, while the trans-isomer (elaidic acid) formed gels at a concentration of 10% w/w. None of the poly-unsaturated fatty acids studied formed gels.

The effect of terminal functional group on gel formation was tested. Gel formation with stearyl amine, stearyl alcohol and stearyl methacrylate was tested. The alcohol formed gels with comparable potency to the acid of the same carbon chain length. The amine formed gels at high concentrations (10% w/w), while the methacrylate was unsuccessful at forming gels at the concentrations tested.

Finally, the gel formation capacity of six waxes that are widely used in the food industry as bland texture manipulating agents or as coating agents³⁵⁻³⁷ was tested. Carnauba wax and candelilla wax were found to be most potent at forming gels (1% w/w). Soy wax was unable to gel the oil even at the highest concentrations tested. Rice bran wax and beeswax had intermediate gelling concentrations.

In summary, the ability to form oil-based gels was dependent on molecular weight, degree of unsaturation, chirality and the functional groups of the various gelling agents.

### Physical Characterization of Oleogels

The gel strength of the oleogels formed using the various gelling agents was compared. Gels formed using saturated fatty acids of various chain lengths was first tested (FIG. 18B). Oleogels formed from palmitic acid (C16) were found to have the greatest gel strength. Interestingly, fatty acids with longer chain lengths such as stearic (C18) and arachidic acid (C20) had a nearly 50% lower gel strength. The G′ value for the largest fatty acid tested [behenic acid (C22)] tested was nearly 2 orders of magnitude lower than palmitic acid (C16). The gel strength of hydroxy fatty acids was then investigated (FIG. 18C), revealing a near opposite trend. The smallest hydroxy fatty acid tested [3-hydroxymyristic acid (C14)] formed the weakest gels, while larger hydroxyl fatty acids formed stronger gels. Although, the location of the hydroxyl group is not comparable across the gelling agents. Next, the effect of terminal functional group on the gel strength of the oleogels was compared. The gel strength of the alcohol containing gelling agent was nearly two-fold higher than that of the acid (FIG. 18D).

Finally, the rheological performance of gels formed using five different waxes was compared (FIG. 18E). Interestingly, it was observed that the various waxes formed gels with G′ values ranging two orders of magnitude. Beeswax, carnauba wax and candelilla wax formed the strongest gels with comparable G′ values. The G′ value of gels formed using castor wax was the lowest, while gels formed using rice bran wax had intermediate G′ values.

Next, the microstructures of the gels formed using various concentrations of rice bran wax and 12-hydroxystearic acid was probed using light microscopy. These two gelling agents were chosen as they formed gels at low concentrations allowing study of a concentration range, and as they represented a natural and synthetic gelling agent. Rice bran wax crystals had a fibrous morphology forming a dendritic interconnected network and their length ranged between 10-20 μm (FIG. 18G). An increase in crystal size was observed with increasing rice bran wax concentration, attributed to the higher available amount of crystalline material facilitating crystal growth, as previously reported ³⁸. At the lowest concentration 12-hydroxystearic acid formed branched dendritic crystals. On increasing the concentration of 12-hydroxystearic acid, crystal arrangement changed dramatically to a rosette-like morphology.

The thermal behaviour of oleogels formed using rice bran wax was then evaluated with differential scanning calorimetry (DSC). The DSC thermogram showed the characteristic melting endothermic peak of rice bran wax at 75° C. and the sequential crystallization of the gelling agent upon cooling at 58° C. (FIG. 18H) highlighting the relative heat-resistance behaviour of the system. In addition, the melting-crystallization transition of rice bran wax is a reversible process, indicating that the system can recover to its initial state after exposure to increased temperatures without any changes in the interaction between the components of the system (FIG. 18F).

Gelling agents were shown to form expansive dendritic microstructures that enabled formation of oleogels. The strength of these gels could be tailored to fit consumer preference or application needs by using different gelling agents.

### Measuring Drug Solubility in Oil-Solubilizer Mixtures

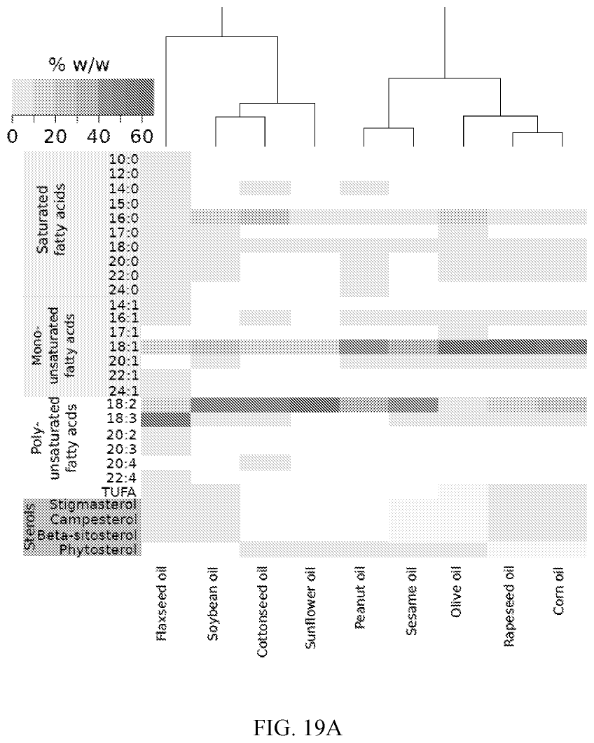

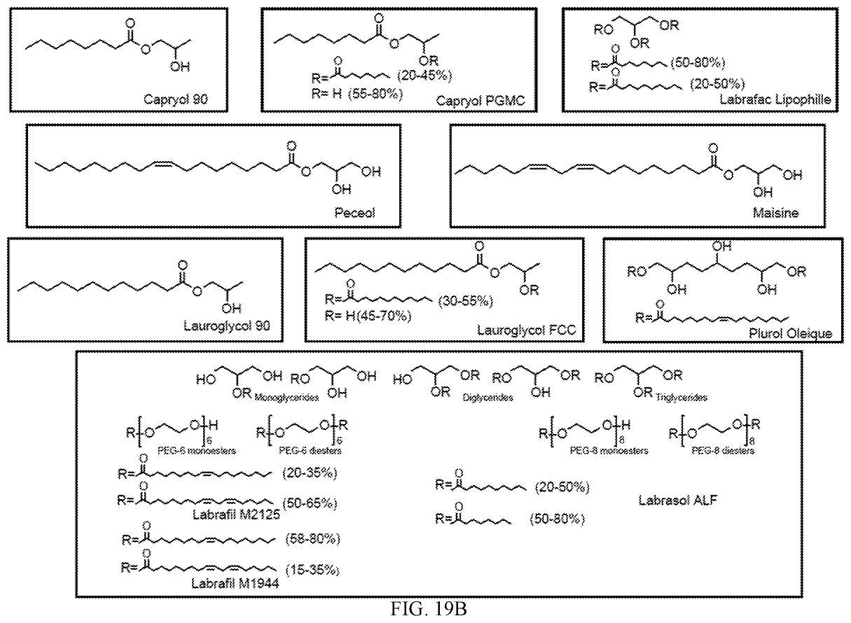

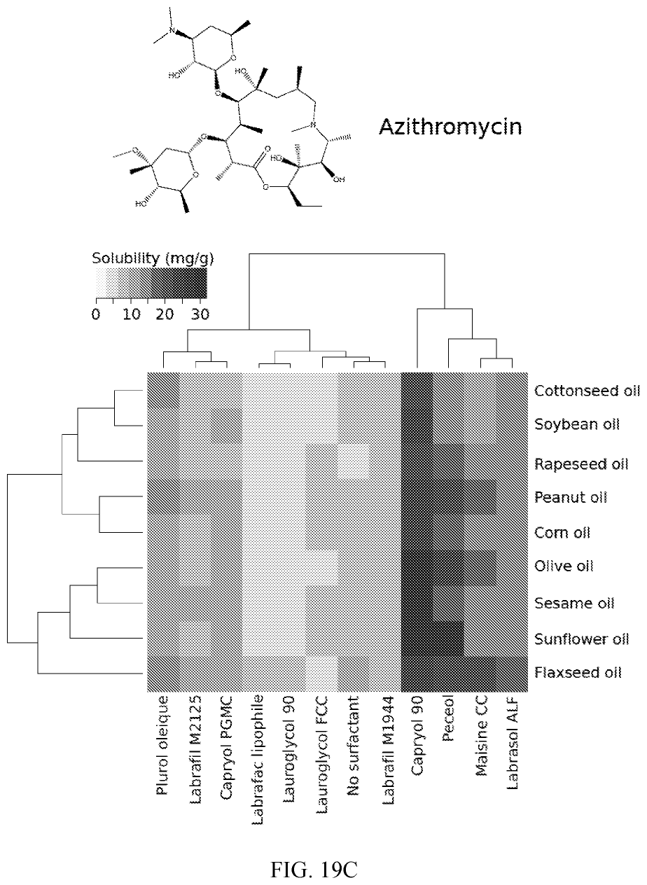

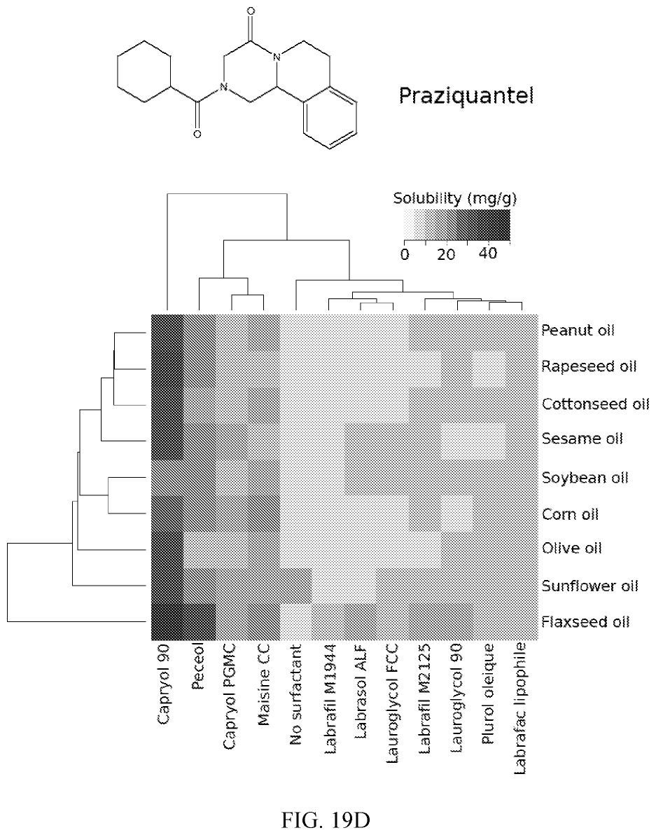

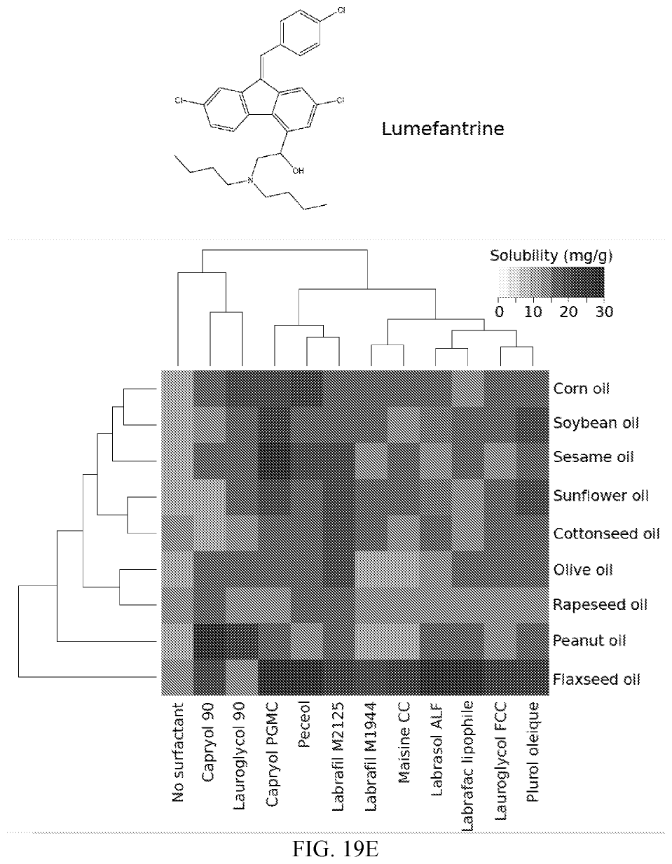

Dissolution is the rate limiting step in the absorption of BCS class II and one of the rate limiting steps for BCS class IV drugs. To maximize drug absorption, design of a drug delivery system that contained the drug in solution was investigated, thereby circumventing the drug dissolution step. To design this formulation, how choice of oil affected drug solubility was first investigated. Nine plant-based oils were selected for these studies (FIG. 19A). The major components of the oils were mono- and di-unsaturated 18-carbon fatty acids. Additionally, the oils contained varying levels of other fatty acids and sterols, which provided a diverse formulation library. To further increase diversity, the oils were mixed with 11 solubilizing agents (FIG. 19B). Solubilizing agents were predominantly fatty acid esters of di- and tri-alcohols. The solubilizing agents have been previously used in foods and FDA-approved drug products, and were used at a concentration comparable to those used in FDA-approved products. As anti-infectives were the most frequently listed drug class on the WHO model list, solubility studies were conducted with three anti-infectives-azithromycin, praziquantel and lumefantrine (FIGS. 19C-19E).

The solubility of azithromycin in the oils was ˜6-10 mg/g (FIG. 19C). Interestingly, addition of solubilizers such as Lauroglycol 90 and Labrafac lipophile led to a slight decrease in solubility. Other solubilizers improved solubility to varying degrees. The maximal increase in solubility was observed with Capryol 90 [31.7±1.6 mg/g (olive oil+Capryol 90) vs. 8.9±0.55 mg/g (olive oil alone), n=3, P<0.05, One-way ANOVA, post-hoc Bonferroni] and Peceol [22.5±0.8 mg/g (olive oil+Peceol) vs. 8.9±0.55 mg/g (olive oil alone), n=3, P<0.05, One-way ANOVA, post-hoc Bonferroni]. In contrast, solubility of azithromycin in water is ˜0.2 mg/g³⁹, nearly 100-fold lower than certain tested formulations.

The solubility of praziquantel in the various oil-solubilizer formulations is shown in FIG. 19D. Praziquantel was soluble up to 5-7 mg/g in most oils. Interestingly, its solubility in sunflower oil was ˜2-fold higher than in the rest of the oils (17.8±2.2 mg/g for sunflower oil vs. 7.2±3.9 mg/g for the oils, n=3, P<0.05, one-sample t-test). Addition of solubilizers led to an increase in solubility of praziquantel. Capryol 90 had maximal impact on drug solubility. Drug solubility in the mixture of Capryol 90 and flaxseed oil was 47.2±3.6 mg/g, n-3 (vs. 6.1±0.8 mg/g in flaxseed oil alone, n=3, P<0.05, two-sample t-test). Interestingly, the solubility of praziquantel in the mixture of Capryol 90 and soybean oil was nearly half of that in the Capryo190+flaxseed oil mixture (23.6±1.3 mg/g vs. 47.2±3.6 mg/g, n=3, P<0.05, two-sample t-test). Hence, choice of both solubilizer and oil achieved maximal solubility.

The solubility of lumefantrine in the oils was highest among the drugs tested (>10 mg/g) (FIG. 19E) and was generally comparable across the oils. Unlike azithromycin and praziquantel, where one/two solubilizers maximally impacted solubility, for lumefantrine there was a small increase in solubility observed with solubilizers. For eight of the eleven solubilizers, maximum solubility was observed when mixed with flaxseed oil. For example, the solubility of lumefantrine in the mixture of Labrafac lipophile and flaxseed oil was 26.6±1.8 mg/g, n=3. In contrast, its average solubility in other formulations containing Labrafac lipophile was ˜1.6-fold lower (16.6±3.8 mg/g, P<0.05, one-sample t-test). Nonetheless, the solubility of lumefantrine observed in formulations tested herein was nearly 10000-fold higher than its reported water solubility (<2 μg/g⁴⁰).

### In Vitro Digestion of Oleogels

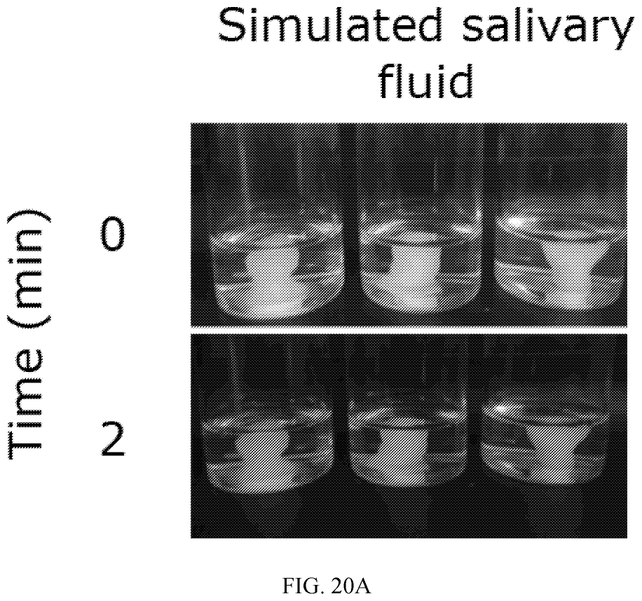

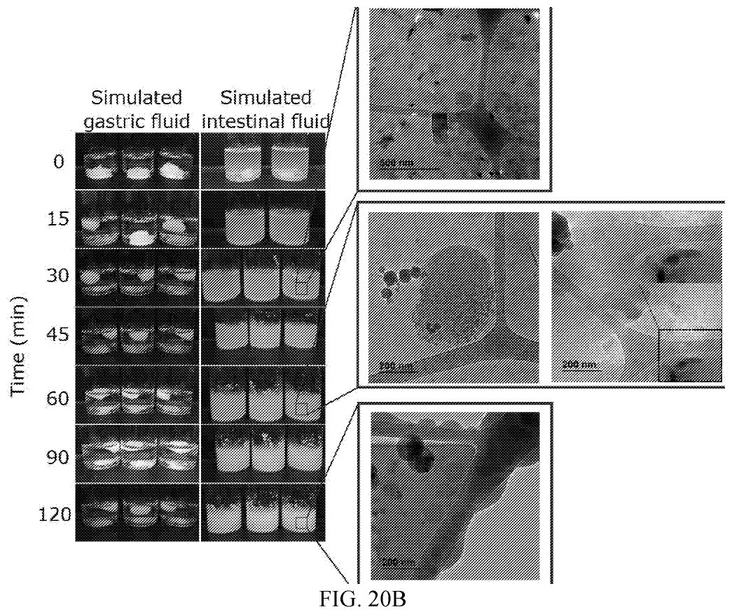

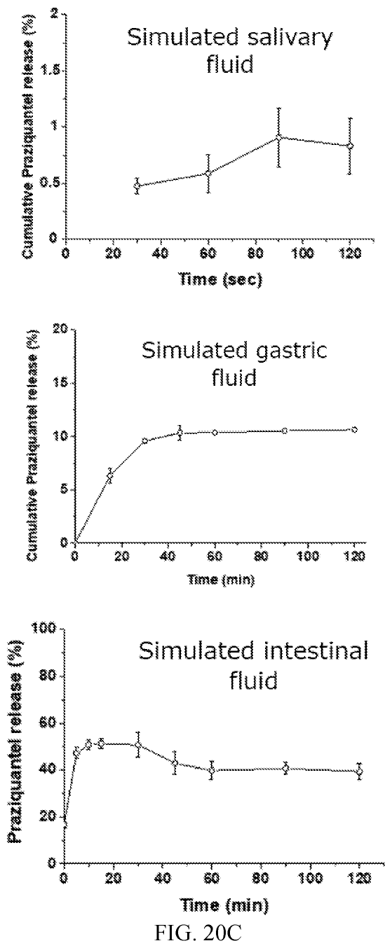

The digestion of oleogels in vitro in simulated salivary, gastric and intestinal conditions were then studied (FIGS. 20A-20B). Incubating the oleogel in simulated salivary and gastric conditions had minimal impact on the overall integrity of the gels. In fact, following 2 h of incubation in simulated gastric fluid, the gel remained phase separated. In contrast, placement of the gel in intestinal fluid led to a rapid disintegration and emulsification of the oleogel (FIG. 20B), which is likely due to the presence of bile salts and surfactants in this medium.

The lipolytic products generated during the digestion of the using cryo-TEM were observed (FIG. 20B, right panel). Different colloidal structures emerged with time. Intact oil droplets and rectangular particles were distinguished during the first 30 min of digestion. The presence of rectangular particles was identified during the initial stage of lipolysis and may be the result of self-association of emulsifying components present in excess in the medium⁴¹. At 60 min, there was a slight decrease in the size of oil droplets, in that droplets of 30-150 nm diameter were observed. Digestion proceeded as mild ‘exfoliation’ of the external layers of the oil droplets, resulting in the formation of lamellar structures with high periodicity. Finally, after 120 min, bilamellar and unilamellar vesicles co-existing with oil droplets and micelles were observed.

The amount of drug released in the various media was then measured (FIG. 20C). Consistent with visual observations, drug release was maximal in simulated intestinal fluid. The amount of praziquantel available in the aqueous phase during simulated gastric digestion reached a maximum 10% of the total drug content within 30 min, attaining a plateau till the end of the 2 h digestion process. The amount of praziquantel transferred in the aqueous simulated intestinal digest was quantified in the mixed micellar phase that represents the bio-accessible fraction of the drug. The solubilized fraction of praziquantel into the aqueous intestinal phase reached 51% at t=15 min. A moderate decrease in praziquantel content in the aqueous phase was observed after 15 min from the initiation of lipolysis, possibly due to a decrease in the solubilization capacity of the digestion medium. The amount of praziquantel detected in the SSF didn't exceed 1% of the total drug content during the time scale of the experiment.

In sum, results presented here indicate the digestion of the formulation under simulated intestinal conditions, while at the same time achieving minimum drug release in the salivary fluid, which is highly desirable in order to minimize drug contact in the mouth cavity and avoid patient aversion, due to the bitter taste of many drug compounds.

### Pharmacokinetics of Oleogels

The pharmacokinetics of the oleogel and tablet formulations of azithromycin, praziquantel and lumefantrine were characterized in a swine model. The tablet was administered orally as is common practice. It was postulated that infants who have difficulty swallowing soft gels may be effectively treated via the rectal route. Hence, the oleogels were administered via both the oral and rectal route. This allowed comparison of the effect of formulation as well as dosing route on the pharmacokinetics.

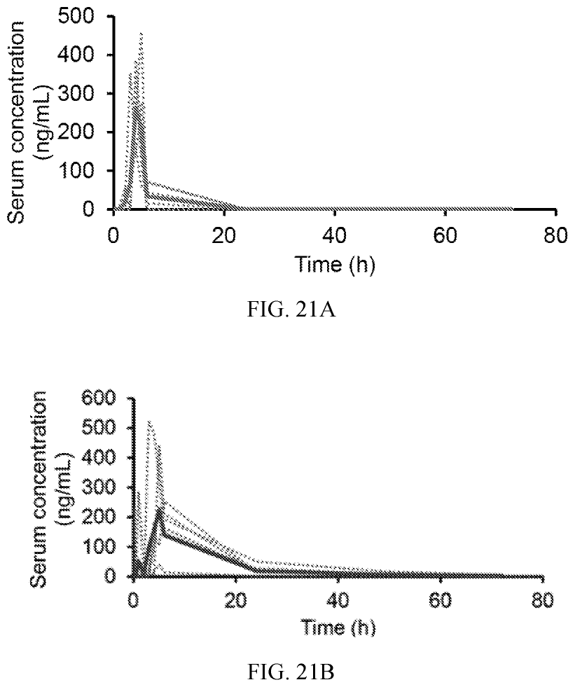

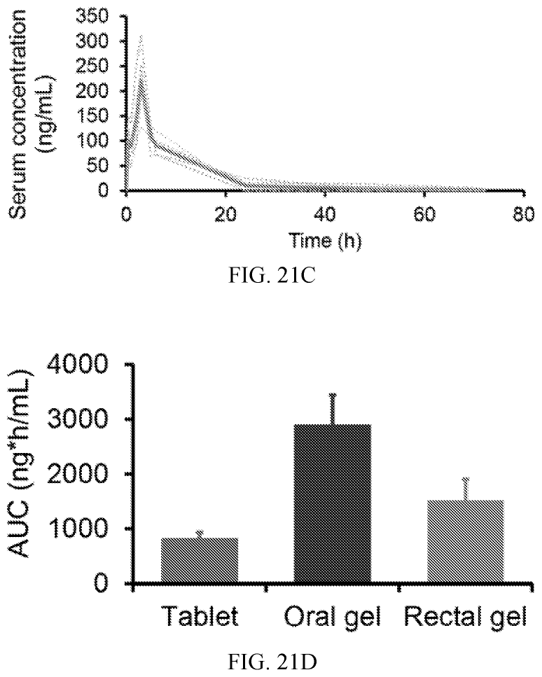

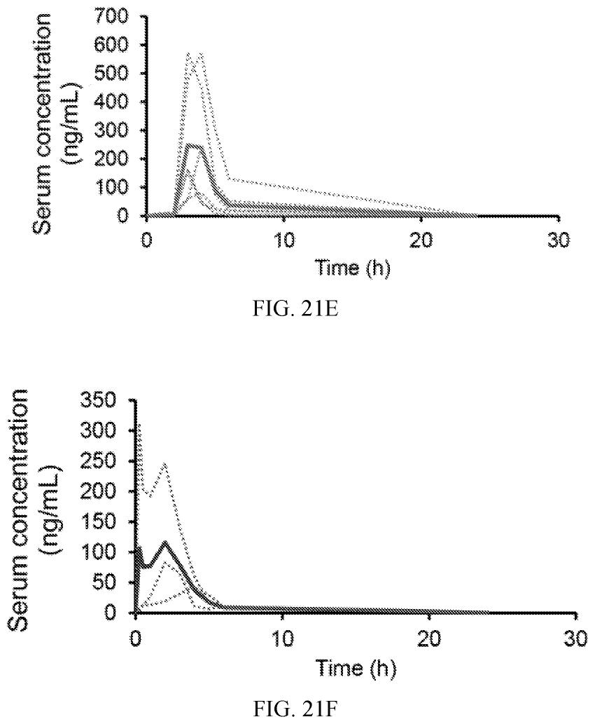

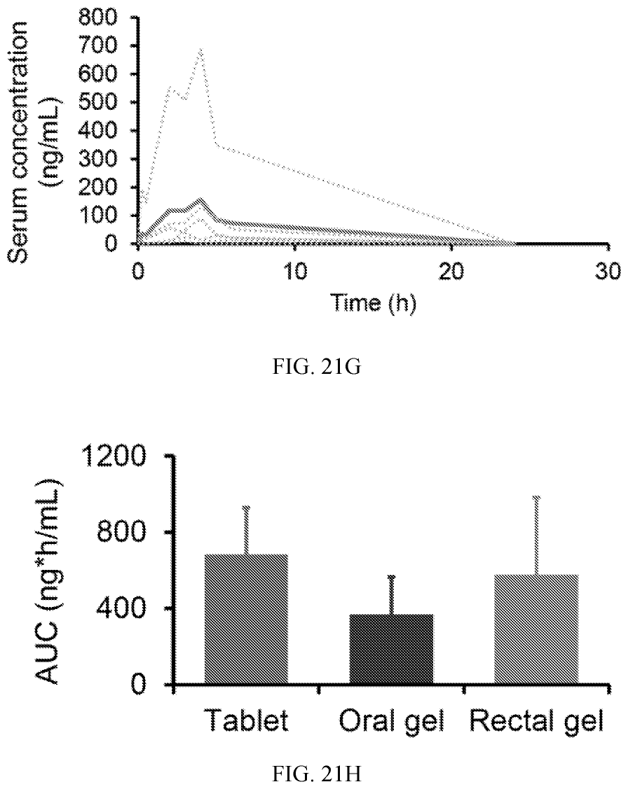

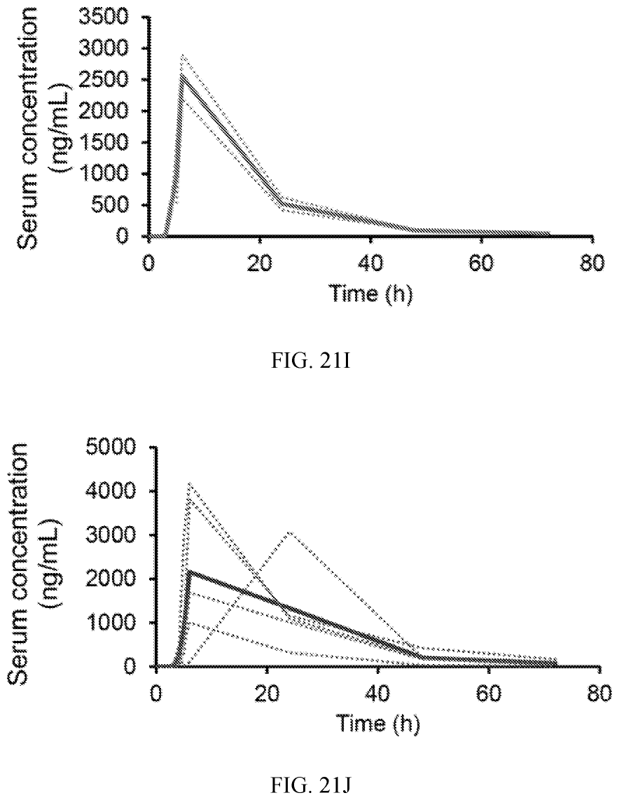

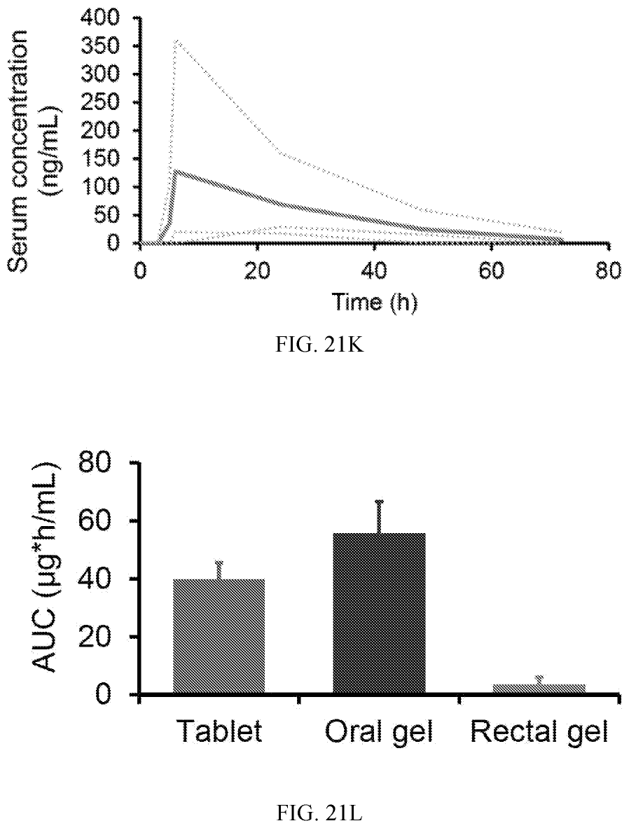

The pharmacokinetics of azithromycin tablets, oral and rectal oleogels are shown in FIGS. 21A-21C. Drug was rapidly absorbed from the formulations, and reached maximal concentrations within 3-4 h. The maximal concentration for the tablet, oral gel and rectal gel were 334±41 ng/mL, 317±54 ng/mL and 224±39 ng/mL (n=4-6) respectively. The bioavailability of azithromycin when dosed as an oral oleogel was nearly 3-fold higher than the tablet (2896±545 ng*h/mL vs. 823±112 ng*h/mL, n=6, *P<0.05, One-way ANOVA, post-hoc Bonferroni). The rectal oleogel resulted in a 2-fold higher bioavailability than the tablet (FIG. 21D).

The pharmacokinetics of praziquantel are shown in FIGS. 21E-21G. Praziquantel was also rapidly absorbed from both the oral and rectal routes. On average, maximal concentrations were observed at 2.1±1.1 h and 3.7±0.4 h for the oral and rectal oleogels. The tₘₐₓ of praziquantel tablets was 3.5±0.2 h. There was no statistical difference between the areas under the curve of praziquantel gels and oral tablets (FIG. 21H), which result in near complete bioavailability⁴².

Finally, the pharmacokinetics of lumefantrine were analyzed upon oral and rectal administration (FIGS. 21I-21K). Regardless of formulation and route of administration, lumefantrine showed a characteristic prolonged half-life. The bioavailability of oral oleogel was comparable to that of the commercial tablet (FIG. 21L). Interestingly, the area under the curve of the rectal oleogel was ˜15-fold lower than the oral oleogel (3389±2617 vs. 55654±10912, n=3-5, *P<0.05, Student's t-test). Without wishing to be bound by theory, the inventors posit that poor partitioning of the drug from the rectal formulation results in lower drug absorption. On the other hand, high solubility of lumefantrine, a weak base, in gastric acid results in complete release from the oral formulation and high drug uptake.

### Single and Multi-Dose Containers for Dispensing Oleogels

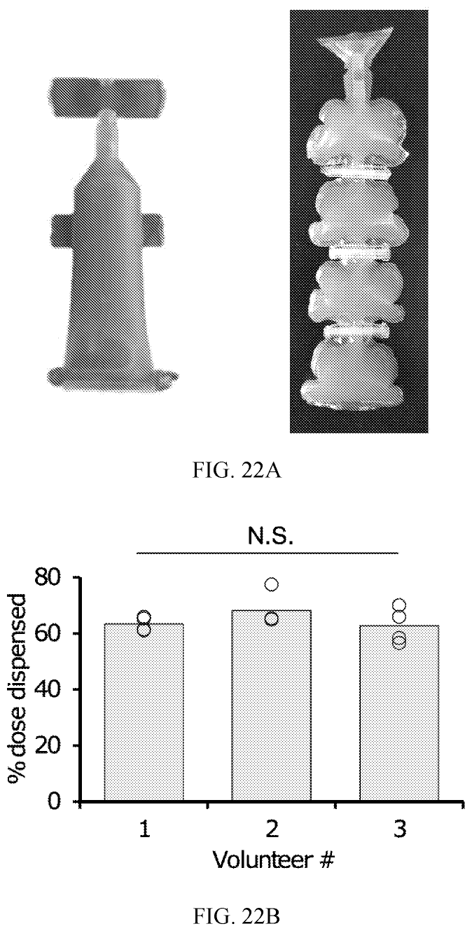

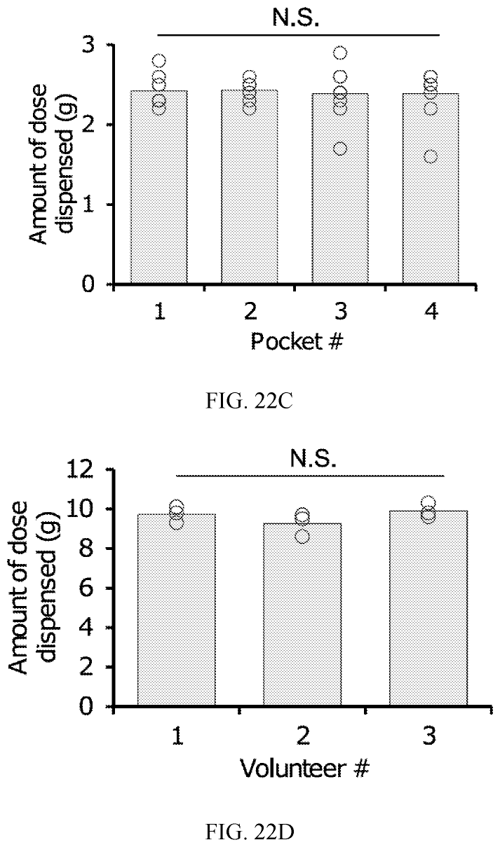

The ideal packaging that will enable easy dispensing and metered dosing of the oleogels was investigated. Two applicator designs were tested—plastic ampoules or unit dose packaging (commercially procured from LF of America) and multi-dose applicators designed and fabricated in-house (FIG. 22A). The uniformity of filling the applicators as well as the reproducibility of dispensing the formulation from the applicators were tested.

Oleogels were hand-filled in 12 single dose containers, and gravimetrically determined consistency in filling. On average, 2.85 g of the oleogel were filled in each container with a standard deviation of 4.5%. Three volunteers were then asked to dispense gels from the containers. Overall, the three individuals were able to dispense 64.7±5.4% (n=12) of the gel was dispensed. The standard deviations for the three individuals dispensing the gels were 2.5% (volunteer 1, n=4), 6.5% (volunteer 2, n=4) and 6.3% (volunteer 3, n=4) (FIG. 22B).

Multi-dose containers were designed to have 4 pockets each, with each pocket sealed off from the next. 10.6±0.3 g (n=9) of the gel was able to be filled in the container. Three volunteers were able to dispense 90.8±5% (n=9) of the formulation from the container, markedly higher than the dispensing from the single dose container. Across three individuals, the average dose dispensed from the four pockets were 2.4±0.2 g (pocket 1, n=9), 2.4±0.1 g (pocket 2, n=9), 2.4±0.3 g (pocket 3, n=9), and 2.4±0.3 g (pocket 4, n=9) (FIG. 22C). Hence, the dispensing across pockets was highly consistent. Finally, quantities of formulation dispersed by the three individuals were 9.7±0.4 g (volunteer 1, n=3), 9.3±0.6 g (volunteer 2, n=3), and 9.9±0.4 g (volunteer 3, n=3) (FIG. 22D), suggesting high consistency even across individuals.

### Discussion

Children are a vulnerable population, especially in low sociodemographic index countries. They are susceptible to diseases due to poor quality of living as well as the unavailability of clean water and adequate nutrition. Safe and efficacious medications that can be administered to children are essential to promote their well-being. However, as children form a small portion of the patient population, most drug products are not optimally designed for their use. Perhaps, the lack of patient volume has also stunted innovation in this field. This compels on-field reformulation of drug products designed for adults by individuals who may not be experts at this craft. Hence, formulations that can be easily administered to children were designed, thereby addressing an unmet clinical need. The formulations described here—oleogels—are inspired by the manipulation of oils described in the food industry. The choice of oleogels as candidate drug carriers was motivated by three factors. First, as most drugs are hydrophobic, oils can serve as an excellent solvent. Second, oils have a long-standing history of use in people, and hence have an established safety profile. Finally, the manufacturing of oleogels is very simple and scalable involving three-unit operations viz. heating, mixing and cooling. Oleogels are amenable to be dosed by both the oral and rectal routes, with the former not involving swallowing of a hard solid. Hence, these have the potential to be used in newborns, infants and children.

Oleogels were composed of three inactive ingredients viz. gelling agents, solubilizers and oils. Here an analysis was performed of the effect of each of these ingredients on the physicochemical properties of the oleogels. It was evident that the choice of gelling agent could affect the viscosity of the formulation, as well as the softening temperature of the formulation. Gelling agents with higher melting temperatures are desired to produce oleogels with high heat stability. However, the use of gelling agents with higher melting points means that the syntheses occur at a higher temperature. This may not be suitable for all drugs. Hence, careful selection of the gelling agent balances long-term physical stability of the formulation, and heat stability of the drug during synthesis. The effect of oil and solubilizer on drug solubility was investigated. It was observed that addition of the solubilizers had a tremendous effect on the solubility of the drug in the oleogel base. It was interesting to note that drug solubility in the oil-solubilizer mixture was not a mere arithmetic sum of its solubility in the individual components. Solubilizers were able to enhance drug solubility in some oils better than in others. The mechanism for this phenomenon was not studied here, but may be of interest in the future. It should be noted that solubilizers were used at different concentrations, that were determined by the maximum levels that they have been used before. This was done to obtain translational information. Future studies analyzing the effect of drug solubility in the various solubilizers, all at the same concentration, may be of interest. A finding of this report is that oleogel formulations of drugs may perform similar to or better than commercial tablets.

### Methods

### Materials

Oils, palmitic acid, 12-hydroxystearic acid, behenic acid, lauric acid, arachidic acid, 12-hydroxylauric acid, linolenic acid, 16-hydroxypalmitic acid, 2-hydroxycaprioic acid, stearyl alcohol, stearyl methacrylate, stearyl amine, oleic acid, stearic acid, glyceryl monooleate [90% United States Pharmacopeia (USP) reference standard (USP)], L-α-phosphatidylcholine [from egg yolk, Type XVI-E, ≥99% (TLC), lyophilized powder], maleic acid (99% pure), sodium taurocholate hydrate, sodium oleate, α-amylase from human saliva, pepsin from porcine gastric mucosa [3200-4500 U mg-1 protein] pancreatin (8×United States Pharmacopeia (USP) specifications) and 4-bromophenylboronic acid (4-BBBA, ≥95.0%) were purchased from Sigma Aldrich. 3-hydroxymyristic acid was purchased from Tokyo Chemicals Industry. Linolenic acid, linoelaidic acid and elaidic acid were purchased from Cayman chemical company. Rice bran wax and castor oil wax was purchased from HalalEveryday. Carnauba wax was purchased from Luxuriant. Beeswax was purchased from Stakich Inc. Candelilla wax was purchased from Plant Guru Inc. Soy wax was obtained from Golden Brands. Lauroglycol FCC, Labrafil M1944, Labrafil M2125, Labrasol ALF, Plurol Oleique, Lauroglycol 90, Labrafac Lipophile, Maisine, Capryol PGMC, Peceol and Capryol 90 were kindly gifted by Gattefossé (Saint Priest, France).

Inversion Assay to Measure Gelling Capacity of Gelling Agents

A variety of gelling agents were mixed with corn oil in increasing concentrations and heated to 5-10° C. above their melting point and then cooled to room temperature. Gel formation was determined using a vial inversion test. Combinations that did not fall to the bottom of the vial were considered gels.

### Rheology

Rheological analysis for the gels was performed on a TA AR2000 rheometer equipped with a 60 mm 2° cone upper geometry with a peltier stage. The gel mixtures were heated beyond their melting point and then transferred onto the peltier stage which was heated to the same temperature. The upper cone was lowered to ensure complete contact with the gel and temperature of the stage was then cooled to 25° C. allowing the melted mixture to congeal. Excess gel was removed using a spatula. Dynamic oscillatory strain amplitude sweep measurements were performed at a frequency of 1 Hz to estimate the viscoelastic region for the gels. After determining the linear viscoelastic region, dynamic oscillatory frequency sweep measurements were performed at 0.01% strain amplitude. For these studies storage modulus (G′) was used as a measure of gel strength. The measurements were analyzed using TA Universal analysis software.

### Brightfield Microscopy

The microstructure of the gels with increasing concentration of gelling agents was examined with light microscopy. A drop of each molten gel sample was deposited on a heated glass slide and slightly pressed with a coverslip to ensure the formation of a thin film. Samples were allowed to cool at ambient temperature for 24 h before being visualized using a Nikon Eclipse ME600 light microscope (Nikon instruments Inc., NY, USA). Images were acquired with a DS-Ri1 camera (Nikon instruments Inc., NY, USA).

### Differential Scanning Calorimetry (DSC)

The thermal behavior (melting and crystallization) of the gel was evaluated using DSC (DSC-8000, Perkin Elmer, USA) equipped with an Intracooler 2 cooling accessory. Data was analyzed using Pyris® (version 11.0.0.0449, Perkin-Elmer) software. Samples in the molten state were weighed in aluminum pans (ca. 5 mg), hermitically sealed and left at ambient temperature for 24 h prior to analysis. Specimen were subjected to three heating/cooling cycles from 20° C. to 120° C. at a rate of 4° C/min and from 120° C. to 20° C. at the same rate under a nitrogen purge of 20 mL/min.

### Measuring Drug Solubility in Oil-Solubilizer Mixtures

Solubility of three anti-infectives, azithromycin, praziquantel and lumefantrine, were measured in 108 formulations prepared by mixing various oils and solubilizers (9 oils×11 solubilizers, and 9 oils without solubilizer). Given the focus in rapid clinical translation, the weight fraction of the solubilizer was limited to the maximum level they were used in FDA-approved products. The concentration of the solubilizers tested is shown in Table 2.1.

TABLE 2.1
List of solubilizers and their levels used in solubility study

| Solubilizer | % w/w |
|---|---|
| Lauroglycol FCC | 0.25 |
| Lauroglycol 90 | 0.25 |
| Labrafil M1944 | 2.94 |
| Labrafac lipophile | 4 |
| Labrasol ALF | 6.9 |
| Labrafil M2125 | 7.5 |
| Plurol Oleique | 8.25 |
| Maisine | 8.6 |
| Peceol | 10 |
| Capryol 90 | 10 |
| Capryol PGMC | 10 |

Approximately two grams of oil-solubilizer mixtures in a 20 mL glass vial. Drug was added in excess to each formulation and the mixture was stirred overnight at room temperature. One milliliter of the mixture was removed, placed in a microcentrifuge tube, and centrifuged at 6000 rpm for 15 min to remove insoluble drug particles. A fraction of the supernant was removed and drug was extracted using methanol or acetonitrile. Drug concentrations in the extracts were measured using HPLC.

An Agilent 1260 Infinity II HPLC system equipped with a quaternary pump, autosampler, thermostat, control module, and diode array detector. Data processing and analysis was performed using OpenLab CDS ChemStation®. Praziquantel chromatographic separations were carried out on an Agilent ZORBAX Eclipse Plus C-18 analytical column (4.6>150 mm) with 5 μm particles, maintained at 40° C. Gradient separation at a flow rate of 1 mL/min was achieved using water and acetonitrile, which corresponds to A and B, respectively. The run time was 5 minutes with a 3-minute post-run and a gradient profile of: 0 min A: 70% and B: 30%, 2.5 min A: 30% and B: 70%. The injection volume was 5 μL. The diode array detector was set using an ultraviolet (UV) detection wavelength of 217 nm with no reference at an acquisition rate of 40 hertz.

Azithromycin chromatographic separations were carried out on an Agilent 4.6×150 mm ZORBAX Eclipse Plus C-18 analytical column with 5 μm particles, maintained at 40° C. Isocratic separation at a flow rate of 1 mL/min was achieved using 10% 10 mM ammonium phosphate dibasic in water and 90% methanol. The run time was 6 minutes. The injection volume was 10 μL. The diode array detector was set using an ultraviolet (UV) detection wavelength of 210 nm with no reference at an acquisition rate of 5 hertz.

Ivermectin chromatographic separations were carried out on a Phenomenex Sphereclone ODS-1 C18 column (4.6×250 mm) with 5 μm particles, maintained at 30° C. Isocratic separation at a flow rate of 0.850 mL/min was achieved using 10% water and 90% methanol. The run time was 15 minutes. The injection volume was 10 μL. The diode array detector was set using an ultraviolet detection wavelength of 254 nm with no reference at an acquisition rate of 10 hertz.

Lumefantrine chromatographic separations were carried out on an Agilent Poroshell 120 PFP column (4.6×100 mm) with 2.7 μm particles, maintained at 40° C. Gradient separation at a flow rate of 1 mL/min was achieved using 0.1% formic acid in water and methanol, which corresponds to A and B, respectively. The run time was 7 minutes with a 3-minute post-run and a gradient profile of: 0 min A: 30% and B: 70%, 3 min A: 5% and B: 95%. The injection volume was 10 μL. The diode array detector was set using an ultraviolet detection wavelength of 303 nm with no reference wavelength at an acquisition rate of 40 hertz.

### In Vitro Digestion and Drug Bioaccessibility Studies

The digestion of gels were characterized in three simulated conditions—oral, gastric and intestinal phases (fasted state).

### Oral Conditions

Simulated salivary fluid (SSF) pH 7.0 was prepared in the presence of a-amylase based on a previously described method^{24,43} with slight modifications. Briefly, SSF stock solution (1.25x concentrated, 3.5 mL) was placed in a glass vial. The 1.25× SSF stock solution contained the following ingredients: 15.1 mmol/L potassium chloride, 3.7 mmol/L potassium dihydrogen phosphate, 13.6 mmol/L sodium bicarbonate, 0.15 mmol/L magnesium chloride and 0.06 mmol/L ammonium carbonate. To this 0.5 mL of a-amylase solution (0.3 mg/mL in SSF stock solution), 0.025 mL of 0.3M calcium chloride and 0.975 mL of water were added and thoroughly mixed.

The SSF was added to 0.5 g praziquantel-loaded gel placed in a pre-heated glass vial at 37° C. containing 5 glass beads to aid in mixing. The vials were placed in an incubator shaker and shaken at 160 rpm and 37° C. One hundred microliters of the sample were withdrawn periodically, centrifuged at 14,000 rpm for 15 min. The supernant was collected and syringe filtered through a 0.22 μm nylon syringe filter (Nalgene Syringe Filters, Thermo Scientific™). Drug concentration in the filtrate was analyzed using HPLC.

### Gastric Conditions

In vitro digestion in gastric phase was performed in 7.5 mL simulated gastric fluid (SGF) pH 2 (2 g/L sodium chloride, 2.917 g/L concentrated hydrochloric acid) in the presence of pepsin (3.4 g/L). SGF was added to 0.5 g gel placed in a pre-heated glass vial at 37° C. containing 5 glass beads to aid in mixing. The vials were shaken as in the oral condition. Samples (0.2 mL) were withdrawn periodically, centrifuged at 14,000 rpm for 15 min and syringe filtered through 0.22 μm nylon syringe filters (Nalgene Syringe Filters, Thermo Scientific™) prior to drug quantification with HPLC.

### Intestinal Phase in Fasted Conditions

The fasting state simulated intestinal fluid version 2 (FaSSIF-V2) medium pH 6.5 was prepared based on a previously described method⁴⁴ in the presence of pancreatin (8×USP specification). To initiate digestion, 50 mL FaSSIF-V2 medium was added in 0.1 g gel placed in a pre-heated glass vial at 37° C. containing 5 glass beads to aid in mixing. Periodically, samples (0.4 mL) were withdrawn and immediately inhibited with the addition of 3 μL of 1M 4-BBBA, prior to ultracentrifugation in polycarbonate tubes for 60 min at 40,000 rpm and 37° C. (ultracentrifuge Optima™ TL with rotor type TLA 120.1, Beckman Coulter, Calif., USA). One hundred microliters were carefully withdrawn from the aqueous micellar phase and diluted with 700 μL of acetonitrile. Samples were then centrifuged for 15 min at 14,000 rpm at room temperature and drug concentration in the supernatant was measured using HPLC.

### Cryogenic Transmission Electron Microscopy of the In Vitro Lipolysis Under FaSSIF Conditions

Visualization of the lipolytic products under fasted state conditions was performed with cryogenic transmission electron microscopy (cryo-TEM). In vitro digestion of the gel was initiated in FaSSIF as described above. Samples were withdrawn at 30 min, 60 min and 120 min, inhibited with the addition of 4-BBBA and immediately processed for cryo-TEM. Three microliters of each sample were deposited on a lacey copper grid coated with a continuous carbon film and blotted to remove excess sample without damaging the carbon layer (Gatan Cryo Plunge III). Grid was mounted on a Gatan 626 single tilt cryo-holder in the TEM column. The specimen and holder tip were cooled down with liquid-nitrogen, which was maintained during transfer into the microscope and subsequent imaging. The images were recorded with a CCD camera (Gatan 2 k×2k UltraScan) on a JEOL 2100 FEG microscope under low dose conditions to avoid sample damage under the electron beam. The microscope was operated at 200 kV and with a magnification in the ranges of 10,000-60,000 for assessing particle size and distribution.

### Oral and Rectal Pharmacokinetics in Pigs

All procedures conformed to the protocols approved by the Massachusetts Institute of Technology Committee on Animal Care.

The pharmacokinetics of azithromycin, praziquantel and lumefantrine were characterized in a large animal model. Female Yorkshire pigs weighting approximately 30-75 kg were fasted overnight and on the day prior to administration with access to water ad libitum. Animals were sedated with intramuscular injection of Telazol (tiletamine/zolazepam) 5 mg/kg, xylazine 2 mg/kg, and atropine 0.04 mg/kg and after intubation, anesthesia was maintained with isoflurane (1-3% inhaled). A central venous catheter was then inserted using the Seldinger technique to allow for frequent blood sampling. Oral administration of the gels was performed in the stomach using an oro-gavage tube with the aid of a syringe. The administered drug dose was 5 mg/kg for azithromycin, 20 mg/kg for praziquantel and 24 mg/kg for lumefantrine. The formulations of azithromycin, praziquantel and lumefantrine gels are shown in Table 2.2. Commercial tablets were administered in gelatin capsules via an oro-gavage tube with 200 mL water using an oral syringe. Rectal gels were dosed with a syringe, inserted 4 inches inside the anal cavity to ensure administration in the rectum. Blood samples were collected in serum separator tubes and centrifuged at 3202 g for 10 min at 4° C. Serum was separated and stored at −80° C. until LC-MS analysis.

TABLE 2.2
Formulations used in pharmacokinetic study

| | |
|---|---|
| Ingredient | Quantity (% w/w) |

Praziquantel oral formulation

| | |
|---|---|
| Praziquantel | 3.8 |
| Ricebran wax | 8 |
| Capryol 90 | 10 |
| Flaxseed oil | q.s. |

Praziquantel rectal formulation

| | |
|---|---|
| Praziquantel | 3.8 |
| Ricebran wax | 6 |
| Capryol 90 | 10 |
| Flaxseed oil | q.s. |

Azithromycin oral formulation

| | |
|---|---|
| Azithromycin dihydrate | 1.78 |
| 1-octadecanol | 8 |
| Capryol 90 | 10 |
| Flaxseed oil | q.s. |

Azithromycin rectal formulation

| | |
|---|---|
| Azithromycin dihydrate | 1.78 |
| 1-octadecanol | 6 |
| Capryol 90 | 10 |
| Flaxseed oil | q.s. |

Lumefantrine oral formulation

| | |
|---|---|
| Lumefantrine | 3.6 |
| Ricebran wax | 8 |
| Capryol 90 | 10 |
| Flaxseed oil | q.s. |

Lumefantrine rectal formulation

| | |
|---|---|
| Lumefantrine | 3.6 |
| Ricebran wax | 6 |
| Capryol 90 | 10 |
| Flaxseed oil | q.s. |

Analyte concentrations in serum were quantified using Ultra-Performance Liquid Chromatography-Tandem Mass Spectrometry (UPLC-MS/MS). Analysis was performed on a Waters ACQUITY UPLC®-I-Class System aligned with a Waters Xevo® TQ-S mass spectrometer (Waters Corporation, Milford MA). Liquid chromatographic separation was performed on an Acquity UPLC® BEH C18 (50 mm×2.1 mm, 1.7 μm particle size) column at 50° C. The mobile phase consisted of aqueous 0.1% formic acid, 10 mM ammonium formate solution (Mobile Phase A) and acetonitrile: 10 mM ammonium formate, 0.1% formic acid solution (95:5 v/v) (Mobile Phase B). The mobile phase had a continuous flow rate of 0.6 mL/min using a time and solvent gradient composition.

For the analysis of praziquantel, the initial composition, 80% Mobile Phase A, was held for 0.50 min, following which the composition was changed linearly to 0% Mobile Phase A over the next 2.00 min. The composition of 0% Mobile Phase A and 100% Mobile Phase B was held constant until 3.50 min. The composition returned to 80% Mobile Phase A at 3.51 min and was held at this composition until completion of the run, ending at 5.00 min, where it remained for column equilibration. The total run time was 5.00 min.

For the analysis of azithromycin, the initial composition, 100% Mobile Phase A, was held for 1.00 min. Following which, the composition was changed linearly to 50% Mobile Phase A and 50% Mobile Phase B until 1.25 min. At 1.50 min the composition was 20% Mobile Phase A. At 2.50 min the composition was 100% Mobile Phase B, where it was held constant until 3.00 min. At 3.25 min the composition returned to 100% A, where it remained for column equilibration for the duration of the run, ending at 4.00 min.

For the analysis of lumefantrine the initial composition of 70% Mobile Phase A was held until 0.50 min. The concentration was changed linearly to 0% Mobile Phase A and 100% Mobile Phase B until 2.50 min, where it was held until 3.50 min. At 3.51 min, the composition returned to 70% Mobile Phase A, where it remained for column equilibration for the remainder of the run. The total run time was 5.00min.

The mass to charge transitions (m/z) used to quantitate praziquantel were 313.22>203.09 and 313.22>83.01 for quantitation and confirmation respectively. For internal standard, mebendazole, 296.06>264.03 and 296.06>76.99 m/z transitions were used for quantitation and confirmation, respectively.

The mass to charge transitions (m/z) used to quantitate azithromycin were 749.732>116.087 and 749.732>83.06 for quantitation and confirmation, respectively. For internal standard, roxithromycin, 837.81>158.14 and 837.81>116.09 m/z transitions were used for quantitation and confirmation, respectively.

The mass to charge transitions (m/z) used to quantitate lumefantrine were 528.28>346.06 and 528.28>276.21 for quantitation and confirmation, respectively. For internal standard, artemisinin, 283.234>247.17 and 283.234>125.135 m/z transitions were used for quantitation and confirmation, respectively.

Sample introduction and ionization was performed by electrospray ionization (ESI) in the positive ionization mode. Waters MassLynx 4.1 software was used for data acquisition and analysis. Stock solutions were prepared in methanol at a concentration of 500m/mL. A twelve-point calibration curve was prepared in analyte-free, blank serum ranging from 1.25-5000 ng/mL. One hundred microliters of each serum sample were spiked with 200 μL of 250 ng/mL internal standard in acetonitrile to elicit protein precipitation. Samples were vortexed, sonicated for 10 min, and centrifuged for 10 min at 13,000 rpm. Two hundred microliters of supernatant were pipetted into a 96-well plate containing 200 μL of water. Finally, 1.00 μL, 10.00 μL, and 2.00 μL were injected onto the UPLC-ESI-MS system for analysis of praziquantel, azithromycin, and lumefantrine, respectively.

### Manufacturing of Multi-Dose Dispenser

To manufacture the multi-dose applicator, three inch thin film PE sleeves (McMaster Can) is used as the base packaging material. A 2D vector design of the applicator was prepared in Adobe Illustrator for laser etching on a 60 W CO2 laser cutter (Universal Laser Systems). The design features four—three mL doses pods that are daisy chained or connected together in a linear fashion with a notched opening at the top for the application. Methods of sealing and resealing was explored including clamping or twisting the channel the separates the pods. The proof of concept was tested using a hermetic zipper aka a ziplock seal. The plastic sleeves are secured down to acrylic sheets using tape and the air is evacuated before sealing the opening of the sleeves. The sleeves are etched using 10P 90S etching settings then removed and excess material is trimmed to form the blank multi-dose applicators. To load the multi-dose applicators, oleogels prepared in syringes were secured to the opening of the applicator with zipties and then dispensed into the applicator until full. The opening of the applicators were sealed with a tabletop impulse sealer (McMaster Can).

The plastic ampoules or unit dose packaging features a blow molded vessel with a one-time use twist off cap. The unit dose applicators were filled in a similar manner to the multi-dosage packaging; three mL of the oleogel were injected into the packaging and then sealed with the tabletop impulse sealer. Both the applicators and syringe were weighed before loading and after loading.

Both packaging forms were tested to understand usability and reproducibility. Three volunteers received four unit-dose applicators and three multi-dose applicators (with four doses total) and instructed to dispense each dose into a weighing boat. The weighing boats were weighed between each dosage and participants were surveyed to understand usability. From the study, participants were able to extract 90.8% of the viscous oleogel from the multi dose applicator while extracting 64.8% of the gel from the unit dose applicator. From surveying the participants, they stated that the thin film multidose applicator did not require as much force to squeeze out the oleogel as compared to the unit dose applicator however the multidose applicator was complex to use. Participants had difficulty extracting the dosages through the linear channels when using the multi-dose applicators. When considering the final packaging form of the oleogels, combining both approaches—thin-film packaging and unit-dose packaging in series—could be considered. Thin-film packaging does not require much force to extraction the viscous oleogels and packaging in series can allow users to tear off the required dosage for a single administration.

### Section 3: Additional Formulations

### Ivermectin

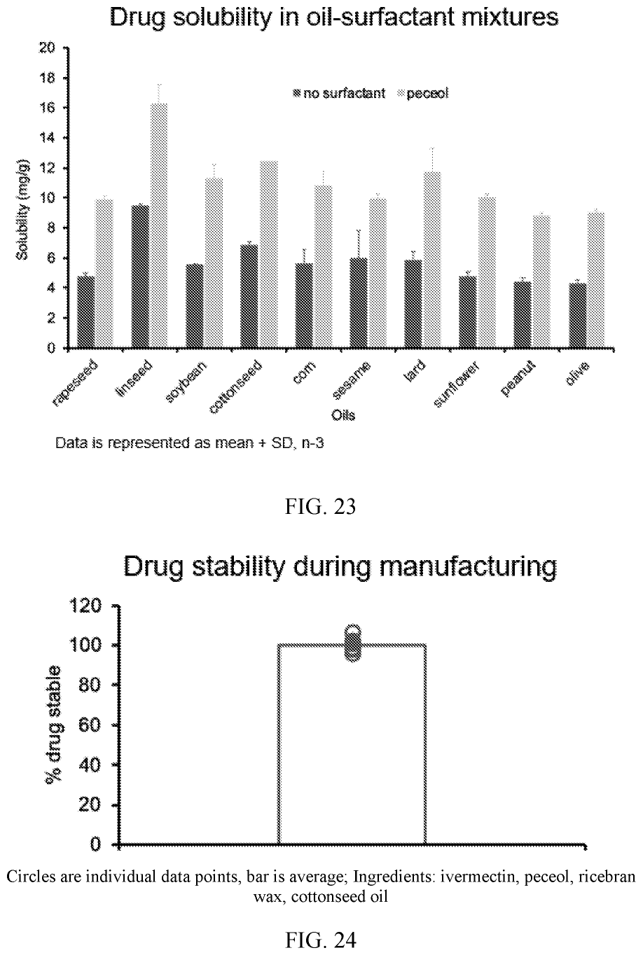

One drug of interest is ivermectin. The solubility of ivermectin in a subset of formulations was measured (see FIG. 23). It was identified that addition of the solubilizer, peceol, improves the solubility of ivermectin in the oil formulations. Cottonseed oil was used due to it being taste neutral as well as peceol for formulating ivermectin.

As manufacturing of the formulation involves heating, the stability of ivermectin during heating was assessed (see FIG. 24). As shown, 100% of the drug was recovered, indicating that the drug is stable during this process.

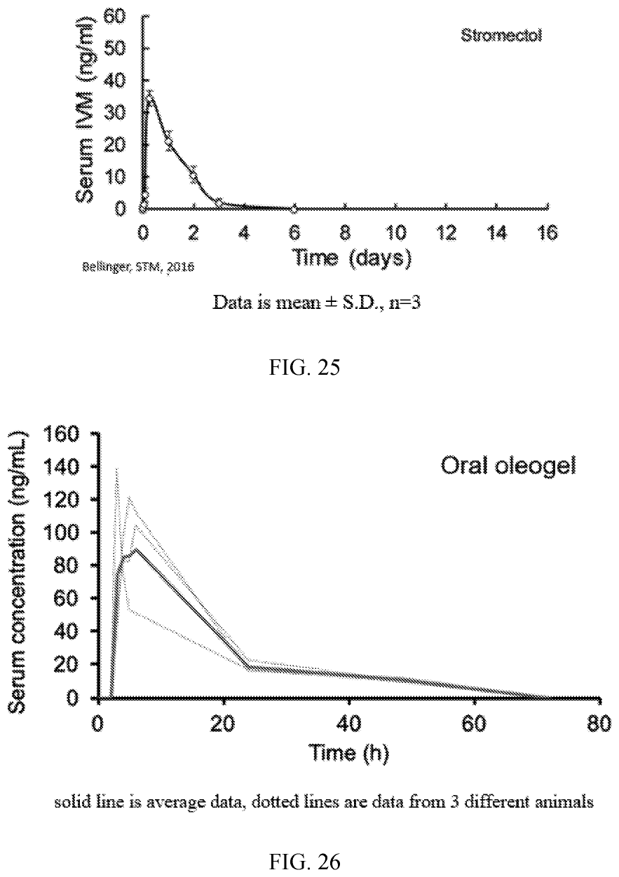

Next, an in vivo study was conducted in pigs to compare the pharmacokinetics of ivermectin oleogel to ivermectin tablets (see FIGS. 25 and 26, Table 3.1). It is evident that the drug is absorbed rapidly from both formulations, and measurable concentrations are observed up to 2 days following dosing. The maximum concentrations observed with the oleogel are 2-3 fold higher than that observed with tablet. The bioavailability (AUC) also is higher with the oleogel than with the tablet.

TABLE 3.1

| | Stromectol | Oleogel |
|---|---|---|
| Cmax (ng/mL) | 35 ± 2 | 122 ± 17 |
| AUC (ng*h/mL) | 1176 ± 264 | 1750 ± 73 |

### Moxifloxcain Oleopastes

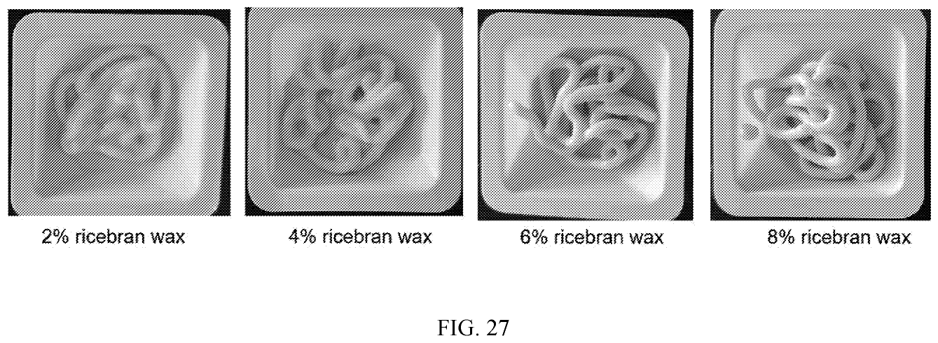

Moxifloxacin was formulated into oleopastes. Oleopastes loaded with 20% moxifloxacin, and with two levels of the gelling agent (2% or 8% rice bran wax) were made. Even at high gelling agent concentrations, the formulations are extrudable. The consistency of the paste can be modified by modulating the concentration of the gelling agent. Shown in FIG. 27 are 4 different oleopastes with the same concentration of moxifloxacin (20%) and formulated with cottonseed oil. However, the amount of gelling agent, ricebran, wax was modulated as indicated in the figure. The formulation with 8% ricebran wax is capable of holding its shape following extrusion from a syringe—indicating its stiffness. While the paste containing least amount of gelling agent (2%) lacks that ability. Regardless of this consistency, the formulations are “soft” and can be easily disfigured like one would a toothpaste.

The two major challenges with suspensions are (1) drug particles settle requiring shaking before administration and (2) as the drug is not solubilized in the formulation, dissolution in the GI fluid may pose a challenge, resulting in low bioavailability. Studies were conducted to determine if any of these are a problem with moxifloxacin oleopaste.

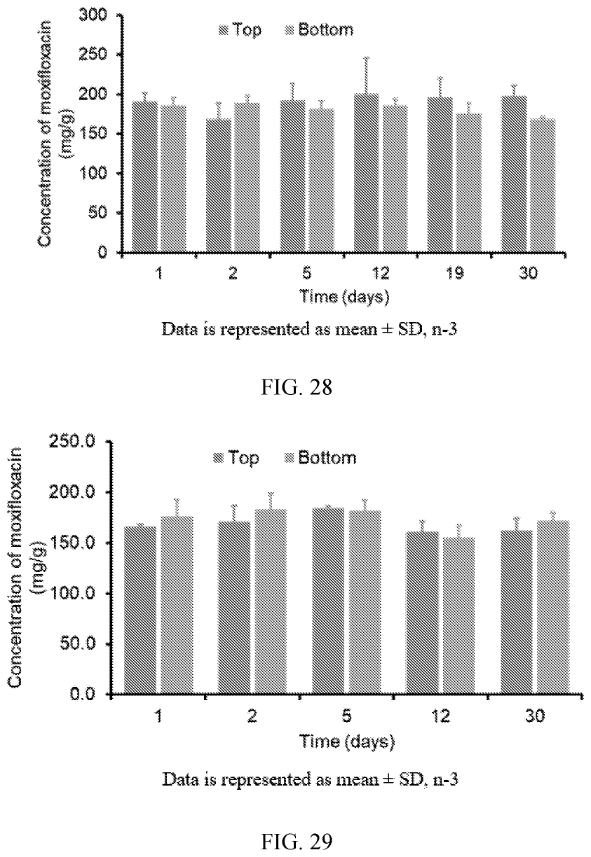

To test whether drug particles settle during storage and shipment, a series of tests were conducted. The oleopaste was prepared and placed in a container for a set time. At each time point, samples were withdrawn from the top and bottom half of the container, drug was extracted with methanol and drug concentration was analyzed. Shown in FIG. 28 are the drug concentrations in the top and bottom half of the formulation when the formulation was stored in a refrigerator (4° C.). The drug concentrations were nearly identical in two halves on day 1, and this similarity was maintained until day 30. This indicated that the particles were homogenously dispersed during its preparation and that there was no settling of the particles over time.

Furthermore, if the formulation was stored at 40° C., the same results were observed (see FIG. 29). The drug concentrations are the same in the top and bottom half of the formulation on day 1, and they remain comparable on day 30.

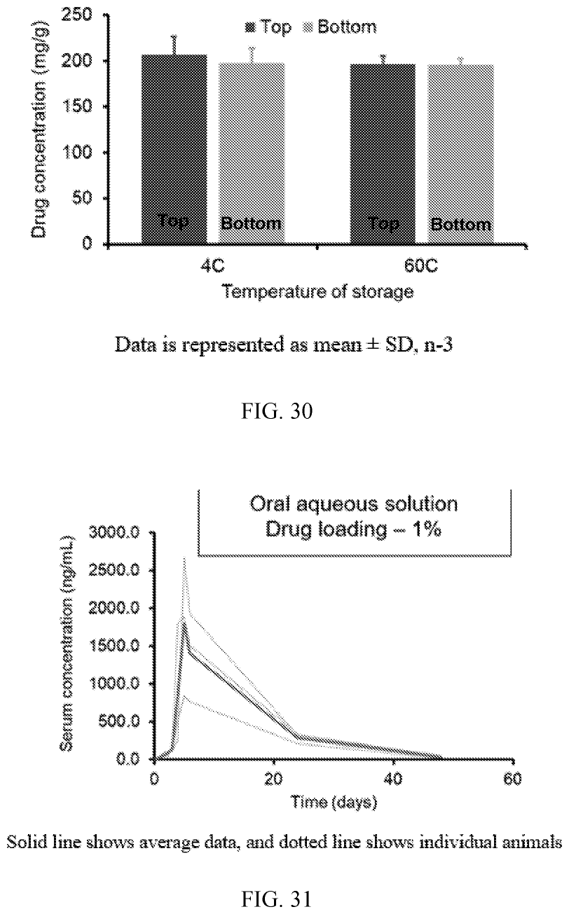

As commerical products can be exposed to temperatures as high as 60° C. for up to a week during transportation, this formulation was exposed to those conditions. The formulation extruded from a syringe after storage at 60° C. and returned to room temperature and a formulation stored in the refrigerator as a control extrude in an analogous way. The chemical stability and homogeneity of the drug contents in these two formulations were tested. It was observed that the drug concentrations in the top and bottom half of the formulations remained the same even when the formulation was stored at 60° C. (see FIG. 30). Additionally, the drug concentrations were the same between the formulation stored at 60° C. and the one stored in a refrigerator. These results indicated that the oleogel formulation base was excellent for maintaining drug particles in suspension for prolonged periods, and under harsh conditions.

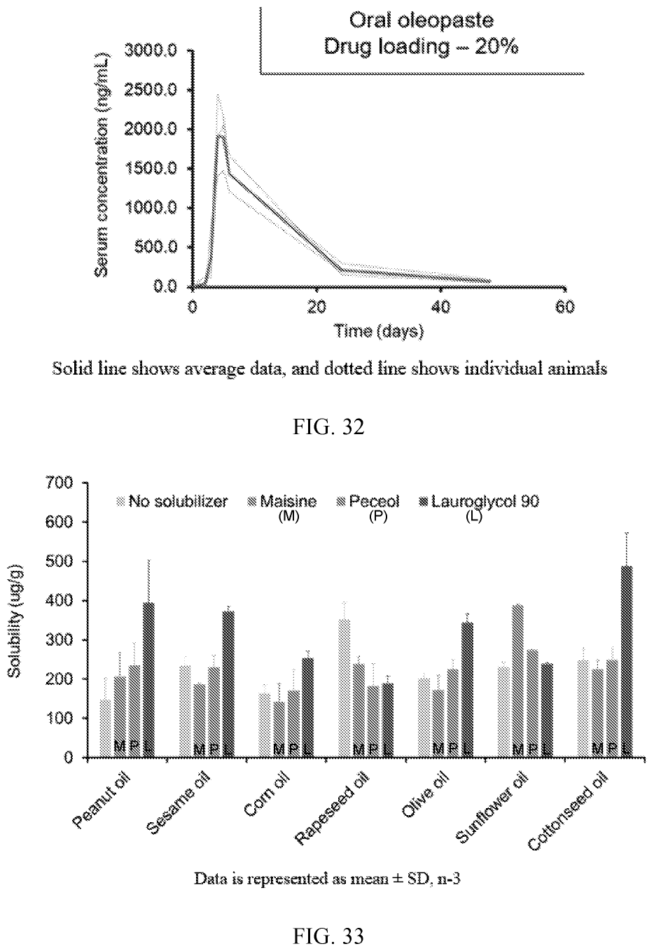

Moxifloxacin formulated as an aqueous solution or an oleopaste was administered orally to pigs. The pharmacokinetics are shown in FIGS. 31 and 32. The drug was absorbed from both formulations. The pharmacokinetics of the oleopaste and the solution were comparable in terms of both the AUC and Cmax (Table 3.2). The solution could be loaded with 1% drug, based on the solubility of moxifloxacin in water. In contrast, the oleopaste is loaded with 20% drug, enabling administration of the same amount of drug as the solution with much less material.

TABLE 3.2

| | Aqueous solution | Oleopaste |
|---|---|---|
| Cmax (ng/mL) | 1809 ± 503 | 1992 ± 280.5 |
| AUC (ug*h/mL) | 23.2 ± 4.7 | 24.2 ± 1.7 |

### Albendazole

One of the drugs pursued for formulation is albendazole. Albendazole was of interest to for two reasons. First, albendazole is a very poorly soluble drug that is available as tablets. In fact, tablets of albendazole are large and children have choked on these tablets before. Additionally, because of these solubility issues, albendazole tablets are recommended to taken with food, which increases its bioavailability by 5-6 fold. In these studies, the hypothesis that the effect of food, and hence dependence on it, may be mitigated when using the oleogel, was tested.

As a first step, the solubility of the drug in 28 formulations was tested, and it was found that its solubility in oil, and oil-solubilizer formulations is ˜10-fold higher than its reported water solubility (41 μg/g) (see FIG. 33). Unfortunately, these solubilities weren't high enough for it to be viable translationally, since at the highest solubility, one would need to ingest ˜1000 g of the formulation to get 400 mg of the drug.

It was then decided to suspend the drug in the oil formulation and make it into an oleopaste by making nanoparticles of albendazole. The inventors posit that making albendazole nanoparticles will afford two advantages: (1) it will allow incorporation of some hydrophilic excipients, which will help with wettability and (2) it will reduce the particle size which will aid in dissolution rate of albendazole in physiological fluids.

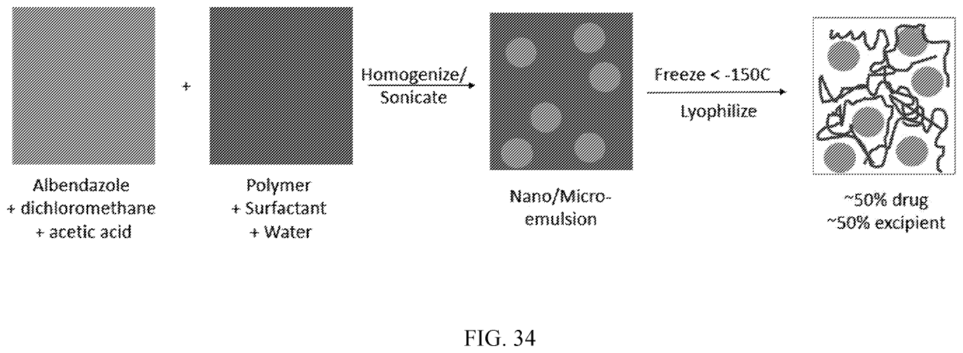

To formulate the nanoparticles on a lab scale, an emulsion of the drug dispersed in an aqueous polymer-surfactant mixture was formed (FIG. 34). Polymers tested include polyvinyl alcohol and hydroxyl propyl methyl cellulose. Surfactants tested include Cremophor EL and Tween 20. Under frozen conditions, the organic solvent was extracted and water was then removed by lyophilization. This provided nanoparticles with a 50% drug loading. Additional strategies such as ball milling or high pressure homogenization are available on an industrial scale to prepare nanoparticles.

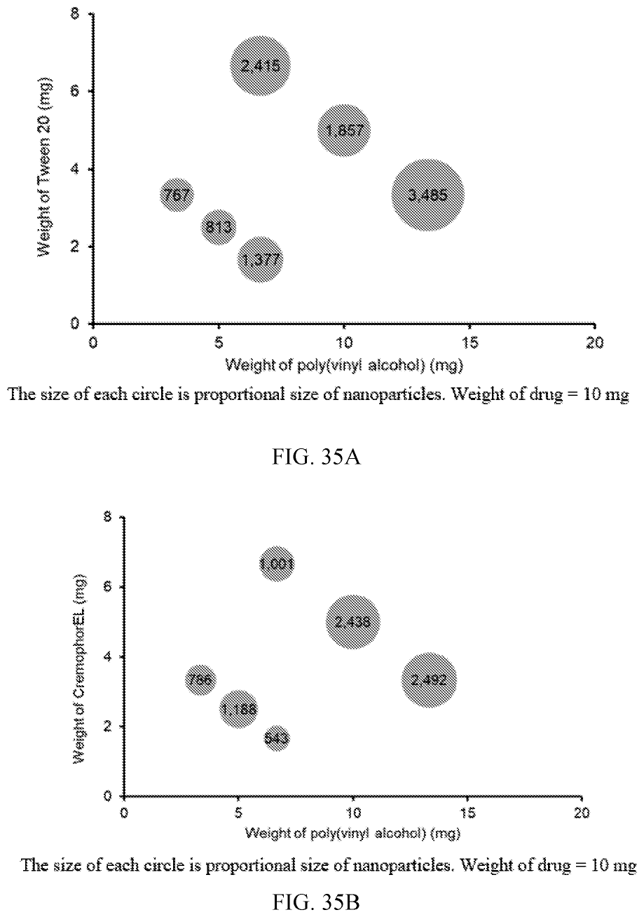

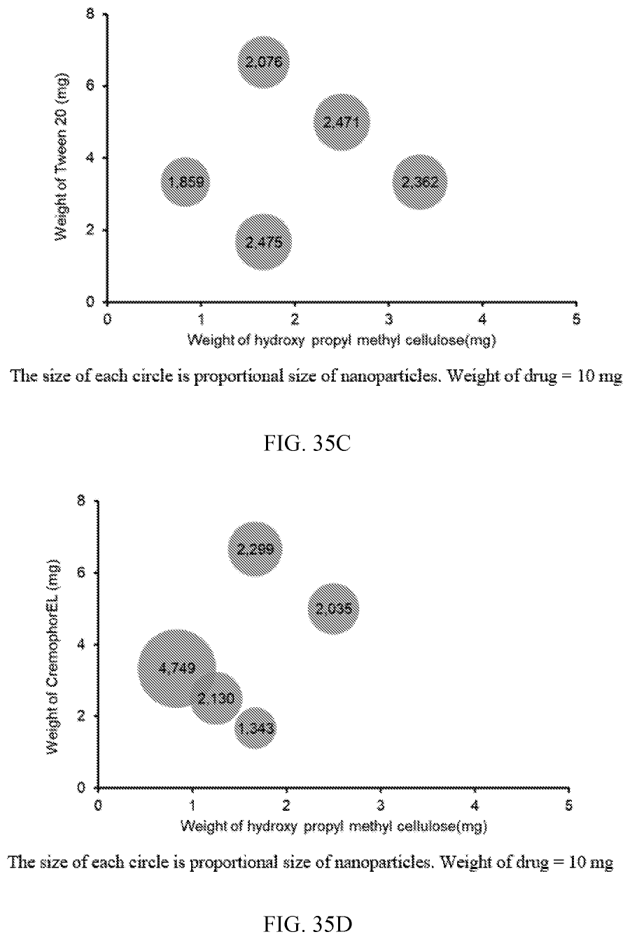

More than 20 formulations were prepared using this strategy. The polymer and surfactant and the quantities of these ingredients used in the formulation were varied. The size of the particles were tested using dynamic light scattering (see FIGS. 35A-35D). 3-4 formulations that were less than a micron a diameter were identified and selected for further development.

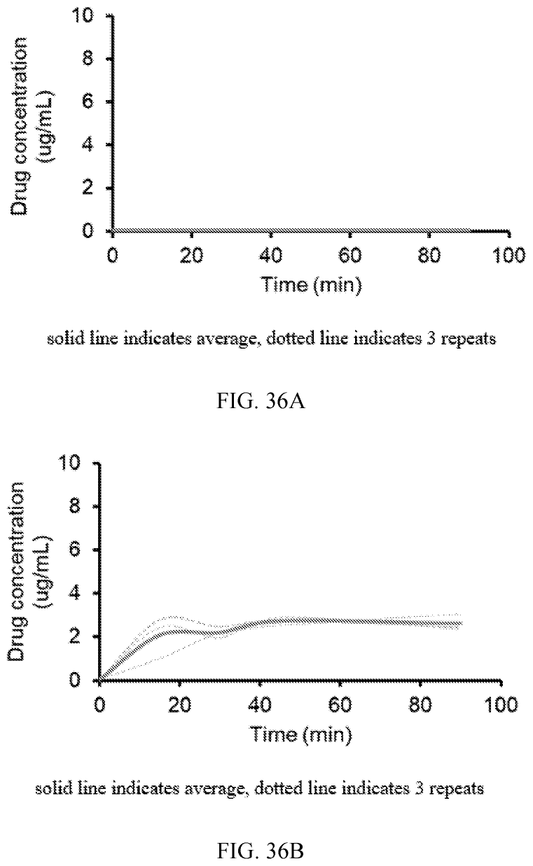

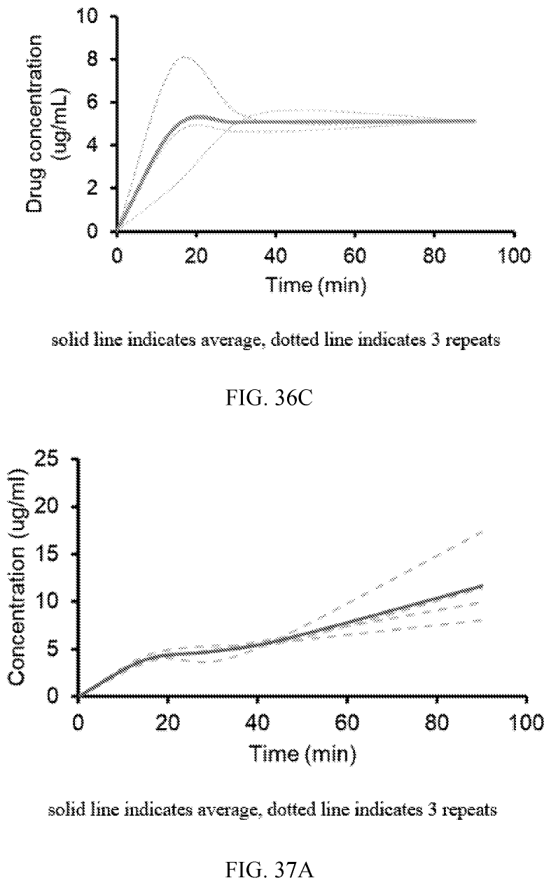

In FIGS. 36A-36C, the release of albendazole either formulated as a powder (FIG. 36A), a physical mixture with a surfactant (Tween 20) and polymer (PVA) (FIG. 36B), or formulated in nanoparticles with the same polymer and surfactant (FIG. 36C) were compared. The formulations were not formulated in oils in these figures. There was no drug release measured for the power. Introduction of the polymer and surfactant led to some drug release (FIG. 36B), however formulating in nanoparticles resulted double the drug release (FIG. 36C).

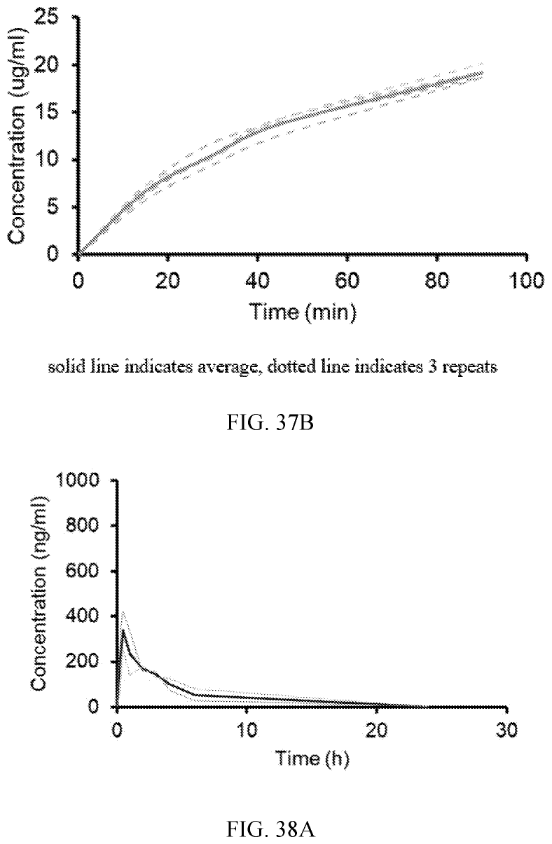

As a next step, the nanoparticles were placed in oil-based gels and analyzed the drug release. In FIGS. 37A and 37B, the rate of drug release from albendazole formulated in oleopaste either as a powder (FIG. 37A) or in the form of nanoparticles (FIG. 37B) was compared. Interestingly, simply putting the albendazole in the oil led to an increased dissolution of the drug in the simulated intestinal fluid (FIG. 37A). Moreover, formulating in the nanoparticles further increased the release rate and quantity from the oil-based formulation (FIG. 37B).

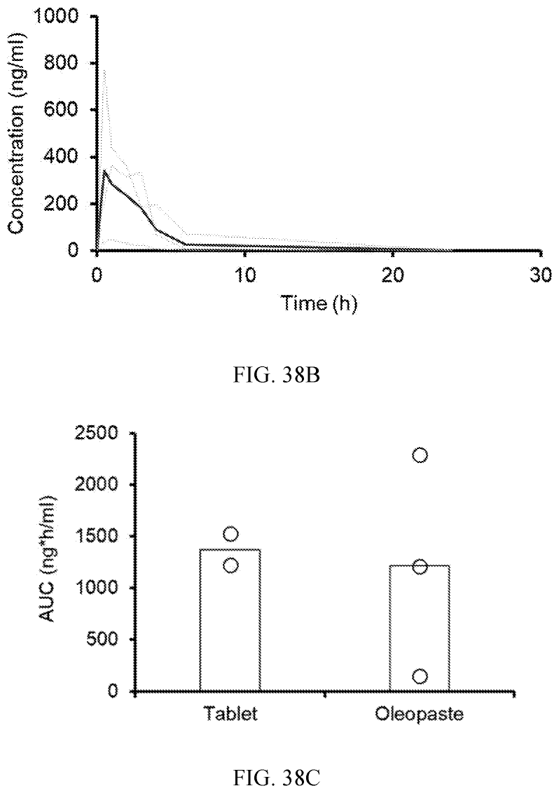

The pharmacokinetics of the commercial tablets of albendazole (FIG. 38A) and oleopaste formulation (FIG. 38B) were tested in rats. Rats were fasted overnight, and then dosed albendazole (5.7 mg/kg) orally. Solid lines are averages, and dotted lines are individual animals. As can be seen in FIGS. 38A-38C, the average serum pharmacokinetics of the two formulations were comparable. There was variability in the pharmacokinetics of the oleopaste (FIG. 38C). There were challenges dosing the oleopaste to rats due to the small volume, which could not be dosed precisely. Notably, this will not be an issue in larger animals. Despite this, the average AUC of the oleopaste and the tablets are comparable.

### Other Formulations

Finally, the stability of drugs in oleogels was tested (Table 3.3). Oleogels loaded with drugs (Azithromycin, Lumefantrine, or Ivermectin) were placed at different temperatures. At various times, drug was extracted from the oleogel and the concentration was measured using HPLC-UV or LC-MS/MS. Drug stability (last column) is represented as mean±S.D., n=5.

TABLE 3.3

| | Conditions | Duration | | % drug |
|---|---|---|---|---|
| Drug name | of storage | of storage | Stabilizer/Antioxidant | stable |
| Azithromycin | 40° C. | 44 days | Propyl gallate | 99 ± 5 |
| Lumefantrine | 60° C. | 28 days | Propyl gallate + Methyl paraben | 94 ± 5 |
| Ivermectin | 40° C. | 28 days | Butylated hydroxyanisole | 98 ± 11 |

### REFERENCES

1. Wang, H. et al. Global, regional, and national under-5 mortality, adult mortality, age-specific mortality, and life expectancy, 1970-2016: a systematic analysis for the Global Burden of Disease Study 2016. Lancet 390, 1084-1150 (2017).
2. Golding, N. et al. Mapping under-5 and neonatal mortality in Africa, 2000-15: a baseline analysis for the Sustainable Development Goals. Lancet 390, 2171-2182 (2017).
3. Liu, L. et al. Global, regional, and national causes of under-5 mortality in 2000-15: an updated systematic analysis with implications for the Sustainable Development Goals. Lancet 388, 3027-3035 (2016).
4. Keenan, J. D. et al. Azithromycin to Reduce Childhood Mortality in Sub-Saharan Africa. N. Engl. J. Med. 378, 1583-1592 (2018).
5. World Health Organization. WHO stresses need to ensure the safety of children's medicines. (2007).
6. Zajicek, A. et al. A report from the pediatric formulations task force: perspectives on the state of child-friendly oral dosage forms. AAPS J. 15, 1072-1081 (2013).

7. Mennella, J. A., Spector, A. C., Reed, D. R. & Coldwell, S. E. The Bad Taste of Medicines: Overview of Basic Research on Bitter Taste. Clin. Ther. 35, 1225-1246 (2013).
8. Organization, W. H. Promoting safety of medicines for children. (2007).
9. Hiller, J. L. et al. Benzyl alcohol toxicity: impact on mortality and intraventricular hemorrhage among very low birth weight infants. Pediatrics 77, 500-6 (1986).
10. Batchelor, H. K. & Marriott, J. F. Formulations for children: problems and solutions. Br. J. Clin. Pharmacol. 79, 405-418 (2015).
11. Richey, R. H. et al. Manipulation of drugs to achieve the required dose is intrinsic to paediatric practice but is not supported by guidelines or evidence. BMC Pediatr. 13, 81 (2013).
12. Zhang, H. et al. Formation and enhanced biocidal activity of water-dispersable organic nanoparticles. Nat. Nanotechnol. 3, 506-511 (2008).
13. Giardiello, M. et al. Accelerated oral nanomedicine discovery from miniaturized screening to clinical production exemplified by paediatric HIV nanotherapies. Nat. Commun. 7, 13184 (2016).
14. Siccardi, M. et al. Towards a rational design of solid drug nanoparticles with optimised pharmacological properties. J. Interdiscip. Nanomedicine 1, 110-123 (2016).
15. Lopez, F. L., Ernest, T. B., Tuleu, C. & Gul, M. O. Formulation approaches to pediatric oral drug delivery: benefits and limitations of current platforms. Expert Opin. Drug Deliv. 12, 1727-1740 (2015).
16. Rogers, M. A. et al. Edible oleogels in molecular gastronomy. Int. J. Gastron. Food Sci. 2, 22-31 (2014).
17. Stortz, T. A. & Marangoni, A. G. Heat resistant chocolate. Trends Food Sci. Technol. 22, 201-214 (2011).
18. Patel, A. R., Cludts, N., Sintang, M. D. Bin, Lesaffer, A. & Dewettinck, K. Edible oleogels based on water soluble food polymers: preparation, characterization and potential application. Food Funct. 5, 2833-2841 (2014).
19. Colla, K., Costanzo, A. & Gamlath, S. Fat Replacers in Baked Food Products. Foods (Basel, Switzerland) 7, 192 (2018).
20. Pavlović, N. et al. Bile acids and their derivatives as potential modifiers of drug release and pharmacokinetic profiles. Front. Pharmacol. 9, 1-23 (2018).
21. Patel, A. R. & Dewettinck, K. Comparative evaluation of structured oil systems: Shellac oleogel, HPMC oleogel, and HIPE gel. Eur. J. Lipid Sci. Technol. 117,1772-1781 (2015).
22. Iwanaga, K., Sumizawa, T., Miyazaki, M. & Kakemi, M. Characterization of organogel as a novel oral controlled release formulation for lipophilic compounds. Int. J. Pharm. 388,123-128 (2010).
23. Lupi, F. R. et al. Olive oil/policosanol organogels for nutraceutical and drug delivery purposes. Food Funct. 4,1512-1520 (2013).
24. O'Sullivan, C. M., Davidovich-Pinhas, M., Wright, A. J., Barbut, S. & Marangoni, A. G. Ethylcellulose oleogels for lipophilic bioactive delivery-effect of oleogelation on in vitro bioaccessibility and stability of beta-carotene. Food Funct. 8,1438-1451 (2017).
25. O'Sullivan, C. M., Barbut, S. & Marangoni, A. G. Edible oleogels for the oral delivery of lipid soluble molecules: Composition and structural design considerations. Trends Food Sci. Technol. 57,59-73 (2016).
26. World Health Organization. WHO Model List of Essential Medicines for Children. (2017). Available at: https://apps.who.int/iris/bitstream/handle/10665/273825/EMLc-6-eng.pdf?ua=1. (Accessed: 2nd July 2019)
27. Kirtane, A. R. et al. Evaluation of Vaginal Drug Levels and Safety of a Locally Administered Glycerol Monolaurate Cream in Rhesus Macaques. J. Pharm. Sci. 106, 1821-1827 (2017).
28. Nel, A. M., Smythe, S. C., Habibi, S., Kaptur, P. E. & Romano, J. W. Pharmacokinetics of 2 Dapivirine Vaginal Microbicide Gels and Their Safety Vs. Hydroxyethyl Cellulose-Based Universal Placebo Gel. JAIDS J. Acquir. Immune Defic. Syndr. 55,161-169 (2010).
29. Stringer, J. L. & Peppas, N. A. Diffusion of small molecular weight drugs in radiation-crosslinked poly(ethylene oxide) hydrogels. J. Control. Release 42,195-202 (1996).
30. Tønnesen, H. H. & Karlsen, J. Alginate in Drug Delivery Systems. Drug Dev. Ind. Pharm. 28,621-630 (2002). 31. Fenton, O. S., Andresen, J. L., Paolini, M. & Langer, R. β-Aminoacrylate Synthetic

Hydrogels: Easily Accessible and Operationally Simple Biomaterials Networks. Angew. Chemie 130,16258-16261 (2018).
32. Jeong, B., Bae, Y. H., Lee, D. S. & Kim, S. W. Biodegradable block copolymers as injectable drug-delivery systems. Nature 388,860-862 (1997).
33. Kumar, S. & Himmelstein, K. J. Modification of in Situ Gelling Behavior of Carbopol Solutions by Hydroxypropyl Methylcellulose. J. Pharm. Sci. 84, 344-348 (1995).
34. Appel, E. A. et al. Self-assembled hydrogels utilizing polymer-nanoparticle interactions. Nat. Commun. 6, 6295 (2015).
35. Zulim Botega, D. C., Marangoni, A. G., Smith, A. K. & Goff, H. D. The Potential Application of Rice Bran Wax Oleogel to Replace Solid Fat and Enhance Unsaturated Fat Content in Ice Cream. J. Food Sci. 78, C1334-C1339 (2013).
36. Talens, P. & Krochta, J. M. Plasticizing Effects of Beeswax and Carnauba Wax on Tensile and Water Vapor Permeability Properties of Whey Protein Films. J. Food Sci. 70, E239-E243 (2006).
37. Saucedo-Pompa, S. et al. Edible film based on candelilla wax to improve the shelf life and quality of avocado. Food Res. Int. 42, 511-515 (2009).
38. Blake, A. I., Co, E. D. & Marangoni, A. G. Structure and Physical Properties of Plant Wax Crystal Networks and Their Relationship to Oil Binding Capacity. J. Am. Oil Chem. Soc. 91, 885-903 (2014).
39. Timoumi, S., Mangin, D., Peczalski, R., Zagrouba, F. & Andrieu, J. Stability and thermophysical properties of azithromycin dihydrate. Arab. J. Chem. 7, 189-195 (2014).
40. Kotila, O. A., Olaniyi, O. O., Adegoke, A. O. & Babalola, C. P. Experimental determination of the physicochemical properties of lumefantrine. Afr. J. Med. Med. Sci. 42, 209-14 (2013).
41. Kuntsche, J., Horst, J. C. & Bunjes, H. Cryogenic transmission electron microscopy (cryo-TEM) for studying the morphology of colloidal drug delivery systems. Int. J. Pharm. 417, 120-137 (2011).
42. McCarthy, J. S. & Moore, T. A. 42 -Drugs for Helminths. in (eds. Bennett, J. E., Dolin, R. & Blaser Douglas, and Bennett's Principles and Practice of Infectious Diseases (Eighth Edition), M. J. B. T.-M.) 519-527.e3 (Content Repository Only!, 2015). doi:https://doi.org/10.1016/B978-1-4557-4801-3.00042-4
43. Minekus, M. et al. A standardised static in vitro digestion method suitable for food—an international consensus. Food Funct. 5, 1113-1124 (2014).
44. Kilic, M. & Dressman, J. A simplified method to screen for in-vivo performance of oral lipid formulations. J. Pharm. Pharmacol. 66, 615-623 (2014).
45. Bavishi, D. D., & Borkhataria, C. H. (2016). Spring and parachute: How cocrystals enhance solubility. Progress in Crystal Growth and Characterization of Materials, 62(3), 1-8. https://doi.org/10.1016/j.perysgrow.2016.07.001
46. Beringer, P., Huynh, K. M. T., Kriengkauykiat, J., Bi, L., Hoem, N., Louie, S., Han, E., Nguyen, T., Hsu, D., Rao, P. A., Shapiro, B., & Gill, M. (2005). Absolute bioavailability and intracellular pharmacokinetics of azithromycin in patients with cystic fibrosis. Antimicrobial Agents and Chemotherapy, 49(12), 5013-5017. https://doi.org/10.1128/AAC.49.12.5013-5017.2005
47. Charalabidis, A., Sfouni, M., Bergström, C., & Macheras, P. (2019). The Biopharmaceutics Classification System (BCS) and the Biopharmaceutics Drug Disposition Classification System (BDDCS): Beyond guidelines. International Journal of Pharmaceutics, 566(May), 264-281. https://doi.org/10.1016/j.ijpharm.2019.05.041
48. Curatolo, W., Liu, P., Johnson, B. A., Hausberger, A., Quan, E., Vendola, T., Vatsaraj, N., Foulds, G., Vincent, J., & Chandra, R. (2011). Effects of food on a gastrically degraded drug: Azithromycin fast-dissolving gelatin capsules and HPMC capsules. Pharmaceutical Research, 28(7), 1531-1539. https://doi.org/10.1007/s11095-011-0386-9
49. Daniel, J., & Rajasekharan, R. (2003). Organogelation of plant oils and hydrocarbons by long-chain saturated FA, fatty alcohols, wax esters, and dicarboxylic acids. JAOCS, Journal of the American Oil Chemists' Society, 80(5), 417-421. https://doi.org/10.1007/s11746-003-0714-0
50. Doan, C. D., To, C. M., De Vrieze, M., Lynen, F., Danthine, S., Brown, A., Dewettinck, K., & Patel, A. R. (2017). Chemical profiling of the major components in natural waxes to elucidate their role in liquid oil structuring. Food Chemistry, 214, 717-725. https://doi.org/10.1016/j.foodchem.2016.07.123
51. Food and Drug Administration. (2011). www.accessdata.fda.gov/drugsatfda_docs/label/. Retrieved from https://www.accessdata.fda.gov/drugsatfda_docs/label/2011/050693s017,050710s033,050 711s031,050784s018,050730s0261b1.pdf
52. Gattefosse. (2020, March 20). Excipient Suppliers List: Gattefossé-Lipid Excipients.

Retrieved from https://www.pharmaexcipients.com/: https://www.pharmaexcipients.com/wp-content/uploads/2020/03/Brochure_Oral-drug-delivery-with-Gattefosse-lipid-excipients.pdf
53. Gigliobianco, M. R., Casadidio, C., Censi, R., & Di Martino, P. (2018). Nanocrystals of Poorly Soluble Drugs: Drug Bioavailability and Physicochemical Stability. Pharmaceutics, 10(3). https://doi.org/10.3390/pharmaceutics10030134
54. Hamm, W., & Hamilton, R. J. (2013). Edible Oil Processing. In Edible Oil Processing (Second). John Wiley & Sons, Ltd. https://doi.org/10.1002/9781118535202
55. Kalepu, S., & Nekkanti, V. (2015). Insoluble drug delivery strategies: Review of recent advances and business prospects. Acta Pharmaceutica Sinica B, 5(5), 442-453. https://doi.org/10.1016/j.apsb.2015.07.003
56. Kauss, T., Gaubert, A., Boyer, C., Ba, B. B., Manse, M., Massip, S., Léger, J. M., Fawaz, F., Lembege, M., Boiron, J. M., Lafarge, X., Lindegardh, N., White, N. J., Olliaro, P., Millet, P., & Gaudin, K. (2013). Pharmaceutical development and optimization of azithromycin suppository for paediatric use. International Journal of Pharmaceutics, 441(1-2), 218-226. https://doi.org/10.1016/j.ijpharm.2012.11.040
57. Kernell, J. W., DePaola, R. V., Maglione, A. M., Ahern, L. N., Penney, N. G., & Addiss, D. G. (2018). Risk of adverse swallowing events and choking during deworming for preschool-aged children. PLoS Neglected Tropical Diseases, 12(6), 1-18. https://doi.org/10.1371/journal.pntd.0006578
58. Kirn, R. B., Wandel, C., Leake, B., Cvetkovic, M., Fromm, M. F., Dempsey, P. J., Roden, M. M., Belas, F., Chaudhary, A. K., Roden, D. M., Wood, A. J. J., & Wilkinson, G. R. (1999). Interrelationship Between Substrates and Inhibitors of Human CYP3A and P-Glycoprotein. Pharmaceutical Research, 16(3), 408-414. https://doi.org/10.1023/A:1018877803319
59. Koradia, D. K., & Parikh, H. R. (2012). Dissolution enhancement of albendazole through nanocrystal formulation. Journal of Pharmacy & Bioallied Sciences, 4(Suppl 1), S62-3. https://doi.org/10.4103/0975-7406.94141
60. Lindenberg, M., Kopp, S., & Dressman, J. B. (2004). Classification of orally administered drugs on the World Health Organization Model list of Essential Medicines according to the biopharmaceutics classification system. European Journal of Pharmaceutics and Biopharmaceutics, 58(2), 265-278. https://doi.org/10.1016/j.ejpb.2004.03.001
61. Lupi, F. R., Gabriele, D., Baldino, N., Mijovic, P., Parisi, O. I., & Puoci, F. (2013). Olive oil/policosanol organogels for nutraceutical and drug delivery purposes. Food and Function, 4(10), 1512-1520. https://doi.org/10.1039/c3fo60259a
62. Matzneller, P., Krasniqi, S., Kinzig, M., Sörge', F., Hüttner, S., Lackner, E., Müller, M., & Zeitlinger, M. (2013). Blood, tissue, and intracellular concentrations of azithromycin during and after end of therapy. Antimicrobial Agents and Chemotherapy, 57(4), 1736-1742. https://doi.org/10.1128/AAC.02011-12
63. Medscape. (2020, May 7). Medscape: Drugs & Diseases: albendazole. Retrieved from albendazole: https://reference.medscape.com/drug/albenza-albendazole-342648
64. Merino, G., Alvarez, A. I., Prieto, J. G., & Kim, R. B. (2002). The Anthelminthic Agent Albendazole Does Not Interact with P-Glycoprotein. Drug Metabolism and Disposition, 30(4), 365 LP-369. https://doi.org/10.1124/dmd.30.4.365
65. Mine, Y., & Zhang, J. W. (2003). Surfactants Enhance the Tight-Junction Permeability of Food Allergens in Human Intestinal Epithelial Caco-2 Cells. International Archives of Allergy and Immunology, 130(2), 135-142. https://doi.org/10.1159/000069009
66. Nalesso, S., Bussemaker, M. J., Sear, R. P., Hodnett, M., & Lee, J. (2019). A review on possible mechanisms of sonocrystallisation in solution. Ultrasonics Sonochemistry, 57(February), 125-138. https://doi.org/10.1016/j.ultsonch.2019.04.020
67. Nichterlein, T., Kretschmar, M., Schadt, A., Meyer, A., Wildfeuer, A., Laufen, H., & Hof, H. (1998). Reduced intracellular activity of antibiotics against Listeria monocytogenes in multidrug resistant cells. International Journal of Antimicrobial Agents, 10(2), 119-125. https://doi.org/10.1016/s0924-8579(98)00030-2
68. Omudhome Ogbru, P. (2020, 3 30). Home: cold and flu health center: cold and flu a-z list: azithromycin article. Retrieved from azithromycin (Zithromax): https://www.medicinenet.com/azithromycin/article.htm#what_is_azithromycin_zithroma x_z-pak
69. O+Sullivan, C. M., Barbut, S., & Marangoni, A. G. (2016). Edible oleogels for the oral delivery of lipid soluble molecules: Composition and structural design considerations. Trends in Food Science and Technology, 57, 59-73. https://doi.org/10.1016/j.tifs.2016.08.018
70. Patel, A. R., Cludts, N., Bin Sintang, M. D., Lewille, B., Lesaffer, A., & Dewettinck, K. (2014). Polysaccharide-based oleogels prepared with an emulsion-templated approach. ChemPhysChem, 15(16), 3435-3439. https://doi.org/10.1002/cphc.201402473
71. Patel, A. R., & Dewettinck, K. (2016). Edible oil structuring: An overview and recent updates. Food and Function, 7(1), 20-29. https://doi.org/10.1039/c5fo01006c
72. Patel, A. R., Rajarethinem, P. S., Cludts, N., Lewille, B., De Vos, W. H., Lesaffer, A., & Dewettinck, K. (2015). Biopolymer-based structuring of liquid oil into soft solids and oleogels using water-continuous emulsions as templates. Langmuir, 31(7), 2065-2073. https://doi.org/10.1021/1a502829u 73. Pehlivano\check{g}lu, H., Demirci, M., Toker, O. S., Konar, N., Karasu, S., & Sagdic, O. (2018). Oleogels, a promising structured oil for decreasing saturated fatty acid concentrations: Production and food-based applications. Critical Reviews in Food Science and Nutrition, 58(8), 1330-1341. https://doi.org/10.1080/10408398.2016.1256866
74. Porter, C. J. H., & Charman, W. N. (1997). Uptake of drugs into the intestinal lymphatics after oral administration. In Advanced Drug Delivery Reviews. https://doi.org/10.1016/S0169-409X(96)00492-9
75. Pouton, C. W. (2006). Formulation of poorly water-soluble drugs for oral administration: Physicochemical and physiological issues and the lipid formulation classification system. European Journal of Pharmaceutical Sciences, 29(3-4 SPEC. ISS.), 278-287. https://doi.org/10.1016/j.ejps.2006.04.016
76. Pubchem. (2020, May 4). Compound summary: Albendazole. Retrieved from National Library of Medicine—National center for Biotechnology Information: https://pubchem.ncbi.nlm.nih.gov/compound/Albendazole
77. Press, D. (2010). Study of Albendazole-Encapsulated Nanosize Liposomes. International Journal, 5, 101-108.
78. Qian, L., & Zhang, H. (2011). Controlled freezing and freeze drying: A versatile route for porous and micro-/nano-structured materials. Journal of Chemical Technology and Biotechnology, 86(2), 172-184. https://doi.org/10.1002/jctb.2495
79. Rawden, H. C., Kokwaro, G. O., Ward, S. A., & Edwards, G. (2000). Relative contribution of cytochromes P-450 and flavin-containing monoxygenases to the metabolism of albendazole by human liver microsomes. British Journal of Clinical Pharmacology, 49(4), 313-322. https://doi.org/10.1046/j.1365-2125.2000.00170.x
80. Romo, M. L., Carpio, A., & Kelvin, E. A. (2014). Routine drug and food interactions during antihelminthic treatment of neurocysticercosis: A reason for the variable efficacy of albendazole and praziquantel? Journal of Clinical Pharmacology, 54(4), 361-367. https://doi.org/10.1002/jcph.269
81. Stieger, N., Joubert, A., & Liebenberg, W. (2017). Solution-mediated crystallization of amorphous azithromycin. Pharmazie, 72(8), 447-448. https://doi.org/10.1691/ph.2017.7035
82. Sugie, M., Asakura, E., Zhao, Y. L., Torita, S., Nadai, M., Baba, K., Kitaichi, K., Takagi, K., Takagi, K., & Hasegawa, T. (2004). Possible Involvement of the Drug Transporters P Glycoprotein and Multidrug Resistance-Associated Protein Mrp2 in Disposition of Azithromycin. Antimicrobial Agents and Chemotherapy, 48(3), 809 LP-814. https://doi.org/10.1128/AAC.48.3.809-814.2004
83. Velík, J., Baliharová, V., Skáková, L., Szotáková, B., Wsól, V., & Lamka, J. (2003).

Stereospecific biotransformation of albendazole in mouflon and rat-isolated hepatocytes. Journal of Veterinary Pharmacology and Therapeutics, 26(4), 297-302. https://doi.org/10.1046/j.1365-2885.2003.00484.x
84. Villaverde, C., Alvarez, A. I., Redondo, P., Voces, J., Del Estal, J. L., & Prieto, J. G.

(1995). Small intestinal sulphoxidation of albendazole. Xenobiotica, 25(5), 433-441. https://doi.org/10.3109/00498259509061863

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A composition comprising:
(a) an active ingredient;
(b) an oil;
(c) a gelling agent; and
(d) optionally, a solubilizing agent,
wherein the composition is in the form of a semisolid dosage form selected from an oleogel and an oleopaste.

2. The composition of claim 1, wherein the oil is a glycerol ester or fatty acid.

3-20. (canceled)

21. The composition of claim 1, wherein the gelling agent is polymer based, a wax, a fatty acid, a hydroxy acid, an unsaturated fatty acid, a saturated fatty acid, a fatty amine, a fatty alcohol, a fatty acrylate, a fatty ester, or a mixture thereof.

22-60. (canceled)

61. The composition of claim 1, wherein the solubilizing agent is a lipophilic surfactant, a fatty acid, a fatty acid ester, or a mixture thereof.

62-83. (canceled)

84. The composition of claim 1, wherein the active ingredient is an active pharmaceutical ingredient, a pesticide, a nutraceutical, or a cosmetic.

85-89. (canceled)

90. The composition of claim 1, wherein the active ingredient is praziquantel, azithromycin, moxifloxacin, ivermectin, lumefantrine, albendazole, or a mixture thereof.

91-103. (canceled)

104. The composition of claim 1, wherein the active ingredient is in the form of a nanoparticle.

105-120. (canceled)

121. The composition of claim 1, wherein the composition further comprises a surfactant.

122. (canceled)

123. The composition of claim 1, wherein the composition further comprises a polymer.

124-127. (canceled)

128. The composition of claim 1, wherein the composition further comprises an antioxidant.

129-133. (canceled)

134. The composition of claim 1, wherein the composition further comprises a flavoring agent.

135-147. (canceled)

148. The composition of claim 1, wherein the composition is formulated for oral, rectal, topical, buccal, mucosal, nasal, intravaginal, intracranial, transdermal, or intraperitoneal administration.

149-185. (canceled)

186. A method of treating a disease or disorder, comprising administering an effective amount of a composition of claim 1 to a subject in need thereof.

187. The method of claim 186, wherein the disease is a cardiovascular disease, cancer, inflammation, hormonal insufficiency, a gastrointestinal disease, malnutrition, a skin disease, poisoning, apnea, glaucoma, an infectious disease, pain, or a neurological disease.

188. (canceled)

189. A method of preventing a disease, comprising administering an effective amount of a composition of claim 1 to a subject in need thereof.

190. A method of delivering an active ingredient, comprising administering an effective amount of a composition of claim 1 to a subject in need thereof.

191. (canceled)

192. A method of overcoming the food effect of an active ingredient, comprising administering an effective amount of a composition of claim 1 to a subject in need thereof.

193-195. (canceled)

196. A kit comprising a composition of claim 1 and instructions for administering the same.

197. The composition of claim 1 made by a process comprising the steps of:
(a) mixing the oil, gelling agent, active ingredient, and optionally, solubilizing agent;
(b) heating the mixture; and
(c) cooling the mixture.

198. A nanoparticle of claim 1 made by a process comprising the steps of:
(a) dissolving the active ingredient in a first solvent system comprising an organic solvent;
(b) emulsifying the dissolved active ingredient in a second solvent system comprising a water, polymer, and a surfactant;
(c) optionally sonicating or agitating the resultant mixture;
(d) freezing the sonicated/agitated mixture; and
(e) lyophilizing the frozen mixture.

199-204. (canceled)
